SEQUENCE LISTING (189 sequences)

SEQ ID NO: 1 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-6-aminohexanoic acid-6-aminohexanoic acid attached to the epsilon-amino group of the lysine side chain
  SITE 30: note = Aminoisobutyric acid
  SITE 35: note = Amidated residue
YXEGTFTSDLSIXLEKQAQXAFVRHLINKXTRQRY

SEQ ID NO: 2 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate attached to the epsilon-amino group of the lysine side chain
  SITE 30: note = Aminoisobutyric acid
  SITE 33: note = N-methyl-L-glutamine
  SITE 35: note = Amidated residue
YXEGTFTSDYSIXLEKQAQXAFVRHLINKXTRQRY

SEQ ID NO: 3 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 35: note = Amidated residue

SEQ ID NO: 4 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKAAQXAFVRHLINKXTRQRY

SEQ ID NO: 5 - protein, 35 aa, synthetic construct
  SITE 2: note = Aminoisobutyric acid
  SITE 13: note = Aminoisobutyric acid
  SITE 16: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEXQAQXAFVRHLINKXTRQRY

SEQ ID NO: 6 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLENQAQXAFVRHLINKXTRQRY

SEQ ID NO: 7 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid attached to the epsilon-amino group of the lysine side chain
  SITE 35: note = Amidated residue
YXEGTFTSDYSIXLEKQAQXAFVRHLINKLTRQRY

SEQ ID NO: 8 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid attached to the epsilon-amino group of the lysine side chain
  SITE 30: note = Aminoisobutyric acid
  SITE 35: note = Amidated residue
YXEGTFTSDYSIXLEKQAQXEFVRHLINKXTRQRY

SEQ ID NO: 9 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-6-aminohexanoic acid attached to the epsilon-amino group of the lysine side chain
  SITE 30: note = Aminoisobutyric acid
  SITE 35: note = Amidated residue

SEQ ID NO: 10 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAEXAFVRHLINKXTRQRY

SEQ ID NO: 11 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine-epsilon-L-ly sine attached to the epsilon-amino group of the lysine side chain
  SITE 30: note = Aminoisobutyric acid
  SITE 35: note = Amidated residue

SEQ ID NO: 12 - protein, 35 aa, synthetic construct; identical to SEQ ID NO: 7
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate attached to the epsilon-amino group of the lysine side chain
  SITE 35: note = Amidated residue

SEQ ID NO: 13 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-6-aminohexanoic acid attached to the epsilon-amino group of the lysine side chain
  SITE 30: note = Aminoisobutyric acid
  SITE 35: note = Amidated residue

SEQ ID NO: 14 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-6-aminohexanoic acid attached to the epsilon-amino group of the lysine side chain
  SITE 30: note = Aminoisobutyric acid
  SITE 35: note = Amidated residue

SEQ ID NO: 15 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDFSIXLEKQAQXAFVRHLINKXTRQRY

SEQ ID NO: 16 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-glutamate-epsilon-L-ly sine attached to the epsilon-amino group of the lysine side chain
  SITE 30: note = Aminoisobutyric acid
  SITE 34: note = De-oxo-arginine
  SITE 35: note = Amidated residue
YXEGTFTSDYSIXLDKQAQXAFVRHLINKXTRQXY

SEQ ID NO: 17 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 33: note = N-methyl-L-glutamine
  SITE 35: note = Amidated residue

SEQ ID NO: 18 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate-epsilon-L-l ysine attached to the epsilon-amino group of the lysine side chain
  SITE 35: note = Amidated residue
YXEGTFTSDYSIXLEKQAQXAFVRHLINKITRQRY

SEQ ID NO: 19 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid attached to the epsilon-amino group of the lysine side chain
  SITE 30: note = Aminoisobutyric acid
  SITE 35: note = Amidated residue

SEQ ID NO: 20 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 21 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDLSIXLEKQAQXAFIRHLINKXTRQRY

SEQ ID NO: 22 - protein, 35 aa, synthetic construct
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDKQAQXAFIRHLINKXTRQRY

SEQ ID NO: 23 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEQQAQXAFVRHLINKXTRQRY

SEQ ID NO: 24 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKIAQXAFVRHLINKXTRQRY

SEQ ID NO: 25 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue
YXEGTFTSDYSIXLEEEAQXAFVRHLINKXTRQRY

SEQ ID NO: 26 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 33: note = N-methyl-L-glutamine
  SITE 35: note = Amidated residue

SEQ ID NO: 27 - protein, 35 aa, synthetic construct; identical to SEQ ID NO: 7
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 35: note = Amidated residue

SEQ ID NO: 28 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKEAQXAFVRHLINKXTRQRY

SEQ ID NO: 29 - protein, 35 aa, synthetic construct
  SITE 2: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 35: note = Amidated residue
YXEGTFTSDYSIALEKQAQXAFVRHLINKXTRQRY

SEQ ID NO: 30 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate-epsilon-L-l ysine attached to the epsilon-amino group of the lysine side chain
  SITE 30: note = Aminoisobutyric acid
  SITE 35: note = Amidated residue

SEQ ID NO: 31 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-tranexamic acid-gamma -L-glutamate-gamma-L-glutamate-gamma-L-glutamate-gamma-L-g lutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 32 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate-epsilon-L-l ysine attached to the epsilon-amino group of the lysine side chain
  SITE 35: note = Amidated residue
YXEGTFTSDYSIXLEKQAQXEFVRHLINKLTRQRY

SEQ ID NO: 33 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDKIAQXAFVRHLINKXTRQRY

SEQ ID NO: 34 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-epsilon -L-lysine-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 35 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-glutamate-epsilon-L-ly sine attached to the epsilon-amino group of the lysine side chain
  SITE 30: note = Aminoisobutyric acid
  SITE 35: note = Amidated residue

SEQ ID NO: 36 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQEQXAFVRHLINKXTRQRY

SEQ ID NO: 37 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFLRHLINKXTRQRY

SEQ ID NO: 38 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 39 - protein, 35 aa, synthetic construct; identical to SEQ ID NO: 32
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 35: note = Amidated residue

SEQ ID NO: 40 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDEQAQXAFVRHLINKXTRQRY

SEQ ID NO: 41 - protein, 35 aa, synthetic construct; identical to SEQ ID NO: 18
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 35: note = Amidated residue

SEQ ID NO: 42 - protein, 35 aa, synthetic construct; identical to SEQ ID NO: 23
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 35: note = Amidated residue

SEQ ID NO: 43 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 34: note = N-methyl-L-arginine
  SITE 35: note = Amidated residue
YXEGTFTSDYSIXLEKQAQXAFVRHLINKXTRQQY

SEQ ID NO: 44 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-6-aminohexanoic acid-6-aminohexanoic acid attached to the epsilon-amino group of the lysine side chain
  SITE 30: note = Aminoisobutyric acid
  SITE 35: note = Amidated residue

SEQ ID NO: 45 - protein, 35 aa, synthetic construct; identical to SEQ ID NO: 32
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-6-aminohexanoic acid attached to the epsilon-amino group of the lysine side chain
  SITE 35: note = Amidated residue

SEQ ID NO: 46 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-diaminobutyric acid attached to the epsilon-amino group of the lysine side chain
  SITE 30: note = Aminoisobutyric acid
  SITE 35: note = Amidated residue

SEQ ID NO: 47 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-8-amino-3,6-dioxaoctanoic acid attached to the epsilon-amino group of the lysine side chain
  SITE 30: note = Aminoisobutyric acid
  SITE 35: note = Amidated residue

SEQ ID NO: 48 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine attached to the epsilon-amino group of the lysine side chain
  SITE 30: note = Aminoisobutyric acid
  SITE 35: note = Amidated residue
YXEGTFTSDYSIXLEEQAQXAFVRHLINKXTRQRY

SEQ ID NO: 49 - protein, 35 aa, synthetic construct; identical to SEQ ID NO: 2
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 35: note = Amidated residue

SEQ ID NO: 50 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 35: note = Amidated residue
YXEGTFTSDYSIXLEKQAQXAFVRQLINKXTRQRY

SEQ ID NO: 51 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 35: note = Amidated residue

SEQ ID NO: 52 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-epsilon- L-lysine-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 53 - protein, 35 aa, synthetic construct
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFIRHLINKXTRQRY

SEQ ID NO: 54 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGEFTSDYSIXLEKQAQXAFVRHLINKXTRQRY

SEQ ID NO: 55 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDAQAQXAFVRHLINKXTRQRY

SEQ ID NO: 56 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate attached to the epsilon-amino group of the lysine side chain
  SITE 30: note = Aminoisobutyric acid
  SITE 35: note = Amidated residue

SEQ ID NO: 57 - protein, 35 aa, synthetic construct
  SITE 2: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 35: note = Amidated residue
YXEGTFTSDYSILLEKQAQXAFVRHLINKXTRQRY

SEQ ID NO: 58 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFVRHLYNKXTRQRY

SEQ ID NO: 59 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-epsilon-L-lysine attached to the epsilon-amino group of the lysine side chain
  SITE 30: note = Aminoisobutyric acid
  SITE 35: note = Amidated residue

SEQ ID NO: 60 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-epsilon-L-lysine-gamma-L-glutamate attached to the epsilon-amino group of the lysine side chain
  SITE 30: note = Aminoisobutyric acid
  SITE 35: note = Amidated residue

SEQ ID NO: 61 - protein, 35 aa, synthetic construct
  SITE 2: note = Aminoisobutyric acid
  SITE 13: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQAAFVRHLINKXTRQRY

SEQ ID NO: 62 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 35: note = Amidated residue

SEQ ID NO: 63 - protein, 35 aa, synthetic construct; identical to SEQ ID NO: 2
  SITE 2: note = Aminoisobutyric acid
  SITE 13: note = Aminoisobutyric acid
  SITE 16: note = Acetyllysine
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 64 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue
YXEGTFTSDYSIXLEEEAQXEFVRHLINKXTRQRY

SEQ ID NO: 65 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLELQAQXAFVRHLINKXTRQRY

SEQ ID NO: 66 - protein, 35 aa, synthetic construct; identical to SEQ ID NO: 18
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue

SEQ ID NO: 67 - protein, 35 aa, synthetic construct
  SITE 2: note = Aminoisobutyric acid
  SITE 13: note = Alpha-methyl-leucine
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDKQAQXAFVRHLINKXTRQRY

SEQ ID NO: 68 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFVRHLIAKXTRQRY

SEQ ID NO: 69 - protein, 35 aa, synthetic construct; identical to SEQ ID NO: 7
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-6-aminohexanoic acid attached to the epsilon-amino group of the lysine side chain
  SITE 35: note = Amidated residue

SEQ ID NO: 70 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine attached to the epsilon-amino group of the lysine side chain
  SITE 30: note = Aminoisobutyric acid
  SITE 35: note = Amidated residue
YXEGTFTSDYSIXLDQQAQXAFVRHLINKXTRQRY

SEQ ID NO: 71 - protein, 35 aa, synthetic construct; identical to SEQ ID NO: 48
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 72 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 34: note = Omega-methyl-L-arginine
  SITE 35: note = Amidated residue

SEQ ID NO: 73 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-8-amino -3,6-dioxaoctanoic acid-8-amino-3,6-dioxaoctanoic acid attached to the epsilon-amino group of the lysine side chain
  SITE 30: note = Aminoisobutyric acid
  SITE 35: note = Amidated residue

SEQ ID NO: 74 - protein, 35 aa, synthetic construct; identical to SEQ ID NO: 67
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 75 - protein, 35 aa, synthetic construct; identical to SEQ ID NO: 48
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 35: note = Amidated residue

SEQ ID NO: 76 - protein, 35 aa, synthetic construct; identical to SEQ ID NO: 53
  SITE 2: note = Aminoisobutyric acid
  SITE 13: note = Alpha-methyl-leucine
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 77 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 78 - protein, 35 aa, synthetic construct; identical to SEQ ID NO: 2
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-glutamate-epsilon-L-ly sine attached to the epsilon-amino group of the lysine side chain
  SITE 30: note = Aminoisobutyric acid
  SITE 35: note = Amidated residue

SEQ ID NO: 79 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 80 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFVRHLLNKXTRQRY

SEQ ID NO: 81 - protein, 35 aa, synthetic construct; identical to SEQ ID NO: 22
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 82 - protein, 35 aa, synthetic construct; identical to SEQ ID NO: 10
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine-epsilon-L-lys ine attached to the epsilon-amino group of the lysine side chain
  SITE 30: note = Aminoisobutyric acid
  SITE 35: note = Amidated residue

SEQ ID NO: 83 - protein, 35 aa, synthetic construct; identical to SEQ ID NO: 32
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue

SEQ ID NO: 84 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAYXAFVRHLINKXTRQRY

SEQ ID NO: 85 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGAFTSDYSIXLEKQAQXAFVRHLINKXTRQRY

SEQ ID NO: 86 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated 4-fluoro-L-phenylalanine
YXEGTFTSDYSIXLEKQAQXAFVRHLINKXTRQRX

SEQ ID NO: 87 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-delta-L-ornithine attached to the epsilon-amino group of the lysine side chain
  SITE 30: note = Aminoisobutyric acid
  SITE 35: note = Amidated residue

SEQ ID NO: 88 - protein, 35 aa, synthetic construct; identical to SEQ ID NO: 53
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 89 - protein, 35 aa, synthetic construct; identical to SEQ ID NO: 48
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 90 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 34: note = De-oxo-arginine
  SITE 35: note = Amidated residue
YXEGTFTSDYSIXLEKQAQXAFVRHLINKXTRQXY

SEQ ID NO: 91 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQRQXAFVRHLINKXTRQRY

SEQ ID NO: 92 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 93 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEATFTSDYSIXLEKQAQXAFVRHLINKXTRQRY

SEQ ID NO: 94 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-epsilon-L-lysine-gamma-L-glutamate attached to the epsilon-amino group of the lysine side chain
  SITE 30: note = Aminoisobutyric acid
  SITE 35: note = Amidated residue

SEQ ID NO: 95 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEGQAQXAFVRHLINKXTRQRY

SEQ ID NO: 96 - protein, 35 aa, synthetic construct; identical to SEQ ID NO: 90
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 34: note = De-oxo-arginine
  SITE 35: note = Amidated residue

SEQ ID NO: 97 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-delta-L-ornithine attached to the epsilon-amino group of the lysine side chain
  SITE 30: note = Aminoisobutyric acid
  SITE 35: note = Amidated residue

SEQ ID NO: 98 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid-8-amino-3,6-dioxaoctanoic acid attached to the epsilon-amino group of the lysine side chain
  SITE 30: note = Aminoisobutyric acid
  SITE 35: note = Amidated residue

SEQ ID NO: 99 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-8-amino-3,6-dioxaoctanoic acid attached to the epsilon-amino group of the lysine side chain
  SITE 30: note = Aminoisobutyric acid
  SITE 35: note = Amidated residue

SEQ ID NO: 100 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue
YXEGTFTSDYSIXLEKEAQXEFVRHLINKXTRQRY

SEQ ID NO: 101 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 102 - protein, 35 aa, synthetic construct; identical to SEQ ID NO: 22
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 103 - protein, 35 aa, synthetic construct; identical to SEQ ID NO: 10
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 104 - protein, 35 aa, synthetic construct; identical to SEQ ID NO: 10
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 105 - protein, 35 aa, synthetic construct; identical to SEQ ID NO: 67
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 106 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-6-aminohexanoic acid-6-aminohexanoic acid attached to the epsilon-amino group of the lysine side chain
  SITE 30: note = Aminoisobutyric acid
  SITE 35: note = Amidated residue

SEQ ID NO: 107 - protein, 35 aa, synthetic construct; identical to SEQ ID NO: 80
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 108 - protein, 35 aa, synthetic construct; identical to SEQ ID NO: 40
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine attached to the epsilon-amino group of the lysine side chain
  SITE 30: note = Aminoisobutyric acid
  SITE 35: note = Amidated residue

SEQ ID NO: 109 - protein, 35 aa, synthetic construct; identical to SEQ ID NO: 53
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 110 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-8-amino -3,6-dioxaoctanoic acid attached to the epsilon-amino group of the lysine side chain
  SITE 30: note = Aminoisobutyric acid
  SITE 35: note = Amidated residue

SEQ ID NO: 111 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAAXAFVRHLINKXTRQRY

SEQ ID NO: 112 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSEXLEKQAQXAFVRHLINKXTRQRY

SEQ ID NO: 113 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine attached to the epsilon-amino group of the lysine side chain
  SITE 30: note = Aminoisobutyric acid
  SITE 35: note = Amidated residue
YXEGTFTSDYSIXLDKQAQXEFVRHLINKXTRQRY

SEQ ID NO: 114 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate attached to the epsilon-amino group of the lysine side chain
  SITE 30: note = Aminoisobutyric acid
  SITE 33: note = N-methyl-L-glutamine
  SITE 35: note = Amidated residue

SEQ ID NO: 115 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDLSIXLEKQAQXAFVRHLLNKXTRQRY

SEQ ID NO: 116 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated N-methyl-L-tyrosine

SEQ ID NO: 117 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid-8-amino-3,6-dioxaoctanoic acid attached to the epsilon-amino group of the lysine side chain
  SITE 30: note = Aminoisobutyric acid
  SITE 35: note = Amidated residue

SEQ ID NO: 118 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-tranexamic acid-gamma- L-glutamate-gamma-L-glutamate-gamma-L-glutamate-gamma-L-gl utamate-gamma-L-glutamate-gamma-L-glutamate-epsilon-L-lysi ne attached to the epsilon-amino group of the lysine side chain
  SITE 30: note = Aminoisobutyric acid
  SITE 35: note = Amidated residue

SEQ ID NO: 119 - protein, 35 aa, synthetic construct; identical to SEQ ID NO: 2
  SITE 2: note = Aminoisobutyric acid
  SITE 13: note = Alpha-methyl-leucine
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 120 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-diaminobutyric acid attached to the epsilon-amino group of the lysine side chain
  SITE 30: note = Aminoisobutyric acid
  SITE 35: note = Amidated residue

SEQ ID NO: 121 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate-epsilon-L-l ysine attached to the epsilon-amino group of the lysine side chain
  SITE 30: note = Aminoisobutyric acid
  SITE 35: note = Amidated residue

SEQ ID NO: 122 - protein, 35 aa, synthetic construct; identical to SEQ ID NO: 57
  SITE 2: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 30: note = Aminoisobutyric acid
  SITE 35: note = Amidated residue

SEQ ID NO: 123 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEAQAQXAFVRHLINKXTRQRY

SEQ ID NO: 124 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLERQAQXAFVRHLINKXTRQRY

SEQ ID NO: 125 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 126 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue
YXEGTFTSDYSIXLEKQAQXAFVRHLINKVTRQRY

SEQ ID NO: 127 - protein, 35 aa, synthetic construct; identical to SEQ ID NO: 7
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue

SEQ ID NO: 128 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFVRALINKXTRQRY

SEQ ID NO: 129 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate attached to the epsilon-amino group of the lysine side chain
  SITE 30: note = Aminoisobutyric acid
  SITE 35: note = Amidated residue
YXEGTFTSDYSIXLEEQAQXEFVRHLINKXTRQRY

SEQ ID NO: 130 - protein, 35 aa, synthetic construct; identical to SEQ ID NO: 64
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-8-amino-3,6-dioxaoctanoic acid-8-amino-3,6-dioxaoctanoic acid attached to the epsilon-amino group of the lysine side chain
  SITE 30: note = Aminoisobutyric acid
  SITE 35: note = Amidated residue

SEQ ID NO: 131 - protein, 35 aa, synthetic construct; identical to SEQ ID NO: 113
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 132 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-epsilon-L-lysine-gamma-L-g lutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 133 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-tranexamic acid-gamma- L-glutamate-gamma-L-glutamate-gamma-L-glutamate-gamma-L-gl utamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 134 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate attached to the epsilon-amino group of the lysine side chain
  SITE 30: note = Aminoisobutyric acid
  SITE 35: note = Amidated residue

SEQ ID NO: 135 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-gamma-L-glutamate-epsilon-L-ly sine attached to the epsilon-amino group of the lysine side chain
  SITE 30: note = Aminoisobutyric acid
  SITE 35: note = Amidated residue

SEQ ID NO: 136 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-8-amino- 3,6-dioxaoctanoic acid-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 137 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-tranexamic acid-gamma -L-glutamate-gamma-L-glutamate-gamma-L-glutamate-gamma-L-g lutamate-gamma-L-glutamate-gamma-L-glutamate-epsilon-L-lys ine attached to the epsilon-amino group of the lysine side chain
  SITE 30: note = Aminoisobutyric acid
  SITE 35: note = Amidated residue

SEQ ID NO: 138 - protein, 35 aa, synthetic construct; identical to SEQ ID NO: 40
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-epsilon-L-lysine attached to the epsilon-amino group of the lysine side chain
  SITE 30: note = Aminoisobutyric acid
  SITE 35: note = Amidated residue

SEQ ID NO: 139 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXAFVRHAINKXTRQRY

SEQ ID NO: 140 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-8-amino-3,6-dioxaoctanoic acid attached to the epsilon-amino group of the lysine side chain
  SITE 30: note = Aminoisobutyric acid
  SITE 35: note = Amidated residue

SEQ ID NO: 141 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine attached to the epsilon-amino group of the lysine side chain
  SITE 30: note = Aminoisobutyric acid
  SITE 35: note = Amidated residue

SEQ ID NO: 142 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYAIXLEKQAQXAFVRHLINKXTRQRY

SEQ ID NO: 143 - protein, 35 aa, synthetic construct; identical to SEQ ID NO: 4
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 144 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQXSFVRHLINKXTRQRY

SEQ ID NO: 145 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 146 - protein, 35 aa, synthetic construct; identical to SEQ ID NO: 90
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 34: note = Beta-homo-L-arginine
  SITE 35: note = Amidated residue

SEQ ID NO: 147 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDRQAQXAFVRHLINKXTRQRY

SEQ ID NO: 148 - protein, 35 aa, synthetic construct; identical to SEQ ID NO: 10
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-8-amino- 3,6-dioxaoctanoic acid-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 149 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 150 - protein, 35 aa, synthetic construct; identical to SEQ ID NO: 2
  SITE 2: note = Aminoisobutyric acid
  SITE 13: note = Alpha-methyl-leucine
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 151 - protein, 35 aa, synthetic construct
  SITE 2: note = Aminoisobutyric acid
  SITE 13: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQYAFVRHLINKXTRQRY

SEQ ID NO: 152 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine-epsilon-L-ly sine attached to the epsilon-amino group of the lysine side chain
  SITE 30: note = Aminoisobutyric acid
  SITE 35: note = Amidated residue

SEQ ID NO: 153 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDKQAQXAFVRYLINKXTRQRY

SEQ ID NO: 154 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-gamma-L-glutamate-6-aminohexa noic acid attached to the epsilon-amino group of the lysine side chain
  SITE 30: note = Aminoisobutyric acid
  SITE 35: note = Amidated residue

SEQ ID NO: 155 - protein, 35 aa, synthetic construct; identical to SEQ ID NO: 40
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-epsilon-L-lysine-epsilon-L-lysine attached to the epsilon-amino group of the lysine side chain
  SITE 30: note = Aminoisobutyric acid
  SITE 35: note = Amidated residue

SEQ ID NO: 156 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSAXLEKQAQXAFVRHLINKXTRQRY

SEQ ID NO: 157 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-6-aminohexanoic acid attached to the epsilon-amino group of the lysine side chain
  SITE 30: note = Aminoisobutyric acid
  SITE 35: note = Amidated residue

SEQ ID NO: 158 - protein, 35 aa, synthetic construct; identical to SEQ ID NO: 7
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate attached to the epsilon-amino group of the lysine side chain
  SITE 35: note = Amidated residue

SEQ ID NO: 159 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue
YXEGTFTSDYSIXLEKQAQXAFVRHLINKATRQRY

SEQ ID NO: 160 - protein, 35 aa, synthetic construct
  SITE 2: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIELEKQAQXAFVRHLINKXTRQRY

SEQ ID NO: 161 - protein, 35 aa, synthetic construct; identical to SEQ ID NO: 10
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-8-amino-3,6-dioxaoctanoic acid-epsilon-L-lysine attached to the epsilon-amino group of the lysine side chain
  SITE 30: note = Aminoisobutyric acid
  SITE 35: note = Amidated residue

SEQ ID NO: 162 - protein, 35 aa, synthetic construct; identical to SEQ ID NO: 10
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-8-amino-3,6-dioxaoctanoic acid-gamma-L-glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 163 - protein, 35 aa, synthetic construct; identical to SEQ ID NO: 8
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-epsilon -L-lysine attached to the epsilon-amino group of the lysine side chain
  SITE 30: note = Aminoisobutyric acid
  SITE 35: note = Amidated residue

SEQ ID NO: 164 - protein, 35 aa, synthetic construct; identical to SEQ ID NO: 28
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 165 - protein, 35 aa, synthetic construct; identical to SEQ ID NO: 29
  SITE 2: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 166 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-epsilon- L-lysine-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 167 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-6-aminohexanoic acid attached to the epsilon-amino group of the lysine side chain
  SITE 30: note = Aminoisobutyric acid
  SITE 35: note = Amidated residue

SEQ ID NO: 168 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-epsilon-L-lysine-gamma-L- glutamate-gamma-L-glutamate-gamma-L-glutamate attached to the epsilon-amino group of the lysine side chain
  SITE 30: note = Aminoisobutyric acid
  SITE 35: note = Amidated residue

SEQ ID NO: 169 - protein, 35 aa, synthetic construct; identical to SEQ ID NO: 1
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 170 - protein, 35 aa, synthetic construct
  SITE 2: note = Aminoisobutyric acid
  SITE 13: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLEKQAQEAFVRHLINKXTRQRY

SEQ ID NO: 171 - protein, 35 aa, synthetic construct; identical to SEQ ID NO: 124
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate-gamma-L-glutamate-epsilon-L-lysine attached to the epsilon-amino group of the lysine side chain
  SITE 30: note = Aminoisobutyric acid
  SITE 35: note = Amidated residue

SEQ ID NO: 172 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 35: note = Amidated residue
YXEGTFTSDYSIXLEKQAQXAFVRHLINKWTRQRY

SEQ ID NO: 173 - protein, 35 aa, synthetic construct
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-gamma-L-glutamate-epsilon-L-lysine attached to the epsilon-amino group of the lysine side chain
  SITE 30: note = Aminoisobutyric acid
  SITE 35: note = Amidated residue
YXEGTFTSDYSIXLDKQAQXAFVRHLLNKXTRQRY

SEQ ID NO: 174 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 17-carboxy-heptadecanoic acid-gamma-L-glutamate-gam ma-L-glutamate-gamma-L-glutamate-gamma-L-glutamate-gamma-L -glutamate-epsilon-L-lysine-gamma-L-glutamate attached to the epsilon-amino group of the lysine side chain
  SITE 30: note = Aminoisobutyric acid
  SITE 35: note = Amidated residue

SEQ ID NO: 175 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-6-aminoh exanoic acid-gamma-L-glutamate-6-aminohexanoic acid attached to the epsilon-amino group of the lysine side chain
  SITE 30: note = Aminoisobutyric acid
  SITE 35: note = Amidated residue

SEQ ID NO: 176 - protein, 35 aa, synthetic construct; identical to SEQ ID NO: 2
  SITE 2: note = Aminoisobutyric acid
  SITE 13: note = Aminoisobutyric acid
  SITE 20: note = Aminoisobutyric acid
  SITE 29: note = 19-carboxy-nonadecanoic acid-gamma-L-glutamate-gamm a-L-glutamate-gamma-L-glutamate-gamma-L-glutamate-gamma-L- glutamate attached to the epsilon-amino group of the lysine side chain
  SITE 30: note = Aminoisobutyric acid
  SITE 35: note = Amidated residue

SEQ ID NO: 177 - protein, 35 aa, synthetic construct
  SITE 1: note = N-methyl-L-tyrosine
  SITE 2: note = Aminoisobutyric acid
  SITE 6: note = Alpha-methyl-phenylalanine
  SITE 10: note = Palmitic acid-GGGG attached to the epsilon-amino group of the lysine side chain
  SITE 13: note = Aminoisobutyric acid
  SITE 20: note = Isovaline
  SITE 30: note = Aminoisobutyric acid
  SITE 35: note = Amidated residue
YXEGTXTSDKSIXLEKQRQXEFVRHLLNKXTRQRY

SEQ ID NO: 178 - protein, 4 aa, synthetic construct
  SITE 1: note = Palmitic acid modified residue
  SITE 4: note = Attached to the epsilon-amino group of a lysine side chain of another sequence
GGGG

SEQ ID NO: 179 - protein, 35 aa, synthetic construct; identical to SEQ ID NO: 177
  SITE 1: note = N-methyl-L-tyrosine
  SITE 2: note = Aminoisobutyric acid
  SITE 6: note = Alpha-methyl-phenylalanine
  SITE 10: note = 17-carboxy-heptadecanoic acid-GGGG attached to the epsilon-amino group of the lysine side chain
  SITE 13: note = Aminoisobutyric acid
  SITE 20: note = Isovaline
  SITE 30: note = Aminoisobutyric acid
  SITE 35: note = Amidated residue

SEQ ID NO: 180 - protein, 4 aa, synthetic construct
  SITE 1: note = 17-carboxy-heptadecanoic acid modified residue
  SITE 4: note = Attached to the epsilon-amino group of a lysine side chain of another sequence
GGGG

SEQ ID NO: 181 - protein, 36 aa, Homo sapiens
YPIKPEAPREDASPEELNRYYASLRHYLNLVTRQRY

SEQ ID NO: 182 - protein, 31 aa, Homo sapiens
HAEGTFTSDVSSYLEGQAAKEFIAWLVKGRG

SEQ ID NO: 183 - protein, 42 aa, Homo sapiens
YAEGTFISDYSIAMDKIHQQDFVNWLLAQKGKKNDWKHNITQ

SEQ ID NO: 184 - protein, 35 aa, synthetic construct
  SITE 2: note = Aminoisobutyric acid
  VARIANT 4: note = A or G
  VARIANT 5: note = A, E, or T
  VARIANT 10: note = F, L, or Y
  VARIANT 11: note = A or S
  VARIANT 12: note = A, E, or I
  VARIANT 13: note = A, aminoisobutyric acid, E, L, or alpha-methyl-leucine
  VARIANT 15: note = D or E
  VARIANT 16: note = A, aminoisobutyric acid, E, G, K, L, acetyllysine, N, Q, or R
  VARIANT 17: note = A, E, I, or Q
  VARIANT 18: note = A, E, or R
  VARIANT 19: note = A, E, Q, or Y
  VARIANT 20: note = A, aminoisobutyric acid, E, or Y
  VARIANT 21: note = A, E, or S
  VARIANT 23: note = I, L, or V
  VARIANT 25: note = A, H, Q, or Y
  VARIANT 26: note = A or L
  VARIANT 27: note = I, L, or Y
  VARIANT 28: note = A or N
  VARIANT 29: note = Lipid moiety comprising a hydrocarbon chain linked via a linker moiety comprising at least one gamma-glutamate residue to the epsilon-amino group of lysine
  VARIANT 30: note = A, aminoisobutyric acid, I, L, V, or W
  VARIANT 33: note = N-methyl-L-glutamine or Q
  VARIANT 34: note = De-oxo-arginine, R, N-methyl-L-arginine, omega-methyl-L-arginine, or beta-homo-L-arginine
  VARIANT 35: note = Amidated 4-fluoro-L-phenylalanine, amidated Y, or amidated N-methyl-L-tyrosine
YXEXXFTSDXXXXLXXXXXXXFXRXXXXKXTRXXX

SEQ ID NO: 185 - protein, 35 aa, synthetic construct; identical to SEQ ID NO: 184
  SITE 2: note = Aminoisobutyric acid
  VARIANT 4: note = A or G
  VARIANT 5: note = A, E, or T
  VARIANT 10: note = F, L, or Y
  VARIANT 11: note = A or S
  VARIANT 12: note = A, E, or I
  VARIANT 13: note = A, aminoisobutyric acid, E, L, or alpha-methyl-leucine
  VARIANT 15: note = D or E
  VARIANT 16: note = A, aminoisobutyric acid, E, G, K, L, acetyllysine, N, Q, or R
  VARIANT 17: note = A, E, I, or Q
  VARIANT 18: note = A, E, or R
  VARIANT 19: note = A, E, Q, or Y
  VARIANT 20: note = A, aminoisobutyric acid, E, or Y
  VARIANT 21: note = A, E, or S
  VARIANT 23: note = I, L, or V
  VARIANT 25: note = A, H, Q, or Y
  VARIANT 26: note = A or L
  VARIANT 27: note = I, L, or Y
  VARIANT 28: note = A or N
  VARIANT 29: note = Lipid moiety HOOC-(CH2)n-CO- wherein n=12-20 linked via a linker moiety comprising at least one gamma-glutamate residue to the epsilon-amino group of lysine
  VARIANT 30: note = A, aminoisobutyric acid, I, L, V, or W
  VARIANT 33: note = N-methyl-L-glutamine or Q
  VARIANT 34: note = Omega-methyl-L-arginine, beta-homo-L-arginine, de-oxo-arginine, N-methyl-L-arginine, or R
  VARIANT 35: note = Amidated 4-fluoro-L-phenylalanine, amidated Y, or amidated N-methyl-L-tyrosine

SEQ ID NO: 186 - protein, 35 aa, synthetic construct
  SITE 2: note = Aminoisobutyric acid
  VARIANT 4: note = A or G
  VARIANT 10: note = F, L, or Y
  VARIANT 11: note = A or S
  VARIANT 13: note = Aminoisobutyric acid or alpha-methyl-leucine
  VARIANT 15: note = D or E
  VARIANT 16: note = K, N, or E
  VARIANT 17: note = A or Q
  VARIANT 18: note = A or R
  VARIANT 19: note = A, E, or Q
  VARIANT 20: note = A, aminoisobutyric acid, or Y
  VARIANT 21: note = A or S
  VARIANT 23: note = I, L, or V
  VARIANT 25: note = A, H, or Y
  VARIANT 27: note = I or L
  VARIANT 29: note = Lipid moiety HOOC-(CH2)n-CO- wherein n=12-20 linked via a linker moiety comprising at least one gamma-glutamate residue to the epsilon-amino group of lysine
  VARIANT 30: note = A, aminoisobutyric acid, I, L, or V
  VARIANT 33: note = N-methyl-L-glutamine or Q
  VARIANT 34: note = De-oxo-arginine or R
  VARIANT 35: note = Amidated 4-fluoro-L-phenylalanine, amidated Y, or amidated N-methyl-L-tyrosine
YXEXTFTSDXXIXLXXXXXXXFXRXLXNKXTRXXX

SEQ ID NO: 187 - protein, 35 aa, synthetic construct
  SITE 2: note = Aminoisobutyric acid
  VARIANT 4: note = A or G
  VARIANT 10: note = F or Y
  VARIANT 11: note = A or S
  VARIANT 12: note = A, E, or I
  VARIANT 13: note = Aminoisobutyric acid or alpha-methyl-leucine
  VARIANT 15: note = D or E
  VARIANT 16: note = K or N
  VARIANT 19: note = A, E, or Q
  VARIANT 20: note = A, aminoisobutyric acid, or Y
  VARIANT 21: note = A or S
  VARIANT 23: note = I, L, or V
  VARIANT 25: note = H or Y
  VARIANT 27: note = I or L
  VARIANT 29: note = Lipid moiety HOOC-(CH2)n-CO- wherein n=12-20 linked via a linker moiety comprising at least one gamma-glutamate residue to the epsilon-amino group of lysine
  VARIANT 30: note = A, aminoisobutyric acid, I, L, or V
  VARIANT 34: note = De-oxo-arginine or R
  VARIANT 35: note = Amidated 4-fluoro-L-phenylalanine or amidated Y
YXEXTFTSDXXXXLXXQAXXXFXRXLXNKXTRQXX

SEQ ID NO: 188 - protein, 35 aa, Synthetic construct; identical to SEQ ID NO: 177
  SITE 2: note = Aminoisobutyric acid
  SITE 6: note = Alpha-methyl-phenylalanine
  SITE 10: note = 17-carboxy-heptadecanoic acid-GGGG attached to the epsilon-amino group of the lysine side chain
  SITE 13: note = Aminoisobutyric acid
  SITE 20: note = isovaline
  SITE 30: note = Aminoisobutyric acid
  SITE 35: note = Amidated residue

SEQ ID NO: 189 - protein, 4 aa, synthetic construct
  SITE 1: note = 17-carboxy-heptadecanoic acid modified residue
  SITE 4: note = Attached to the epsilon-amino group of a lysine side chain of another sequence
GGGG

(19) United States
(12) Patent Application Publication
(10) Pub. No.: US 2025/0382344 A1
(43) Pub. Date: Dec. 18, 2025

(54) Soluble GLP1/GIP/NPY2 Receptor Triple Agonists

(71) Applicant: Boehringer Ingelheim International GmH, Ingelheim am Rhein (DE)

(72) Inventors: Peter Wilhelm HAEBEL, Mittelbiberach (DE); Charlotte Stahl MADSEN, Maaloev (DK); Anthony MURRAY, Charlottenlund, Gentofte (DK); Alexander WEBER, Biberach an der Riss (DE)

(21) Appl. No.: 19/235,851

(22) Filed: Jun. 12, 2025

(30) Foreign Application Priority Data
Jun. 13, 2024 (EP) 24181845.9

(51) Int. Cl. C07K 14/575; A61K 38/00; C07K 14/605

(52) U.S. Cl. CPC C07K 14/575; C07K 14/605; A61K 38/00

(57) ABSTRACT

The present invention relates to peptide compounds that agonize the GIP, GLP-1 and neuropeptide Y2 (NPY2) receptors, agonists, processes for their preparation, pharmaceutical compositions thereof, and their medical use in the treatment and/or prevention of a variety of diseases such as obesity, diabetes, metabolic syndrome, and/or MASH/NASH. The peptide compounds of the invention are suitable for subcutaneous administration.

### RELATED APPLICATION DISCLOSURE

This application claims the benefit pursuant to 35 U.S.C. § 119 of European Patent Application No. 24181845.9, filed on 13 Jun. 2024, pending, which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3584-US-1_Sequence-Listing_SL.xml” created on May 8, 2025, having a size of 638,448 bytes, which is hereby incorporated by reference herein in its entirety.

### FIELD OF THE INVENTION

The present invention relates to peptide compounds that agonize the GIP, GLP-1 and neuropeptide Y2 (NPY2) receptors and to their medical use in the treatment and/or prevention of a variety of diseases, conditions or disorders, such as obesity, diabetes, metabolic syndrome, and/or MASH/NASH.

### BACKGROUND INFORMATION

Overweight and obesity are defined as abnormal or excessive fat accumulation that presents a risk to health. A body mass index (BMI) over 27 kg/m² is considered as overweight, and a BMI over 30 kg/m² is considered as obese. The BMI is calculated based on body weight and height. Obesity has in the past 50 years reached pandemic levels. Worldwide obesity has nearly tripled since 1975. In 2016, more than 1.9 billion adults and more than 340 million children and adolescents were overweight or obese. Both overweight and obesity are a major risk factor for several chronic diseases, including type 2 diabetes, cardiovascular diseases and cancer which are the leading causes of morbidity and death in many countries. Obesity is thus a serious condition and associated with poorer mental health outcomes, reduced quality of life, and contributes to a decline in life expectancy.

Peptide YY (PYY) is a 36-amino acid peptide with the sequence YPIKPEAPREDASPEELNRYYASLRHYLNLVTRQRY (SEQ ID NO: 181), found in endocrine L cells in the mucosa of the gastrointestinal tract, especially in the ileum and colon. PYY belongs to the pancreatic polypeptide (PP) family together with neuropeptide Y (NPY) and pancreatic polypeptide (PP). This family of peptides act upon the NPY receptors designated NPY1R (Y1), NPY2R (Y2), NPY4R (Y4), and NPY5R (Y5). The receptor family belongs to a class of G protein-coupled receptors (GPCRs) that are expressed in the CNS, especially in regions of the hypothalamus. The receptors NPY1R-NPY5R exhibit both anorectic (NPY2R, NPY4R) and orexigenic effects (NPY5R, NPY1R). The two major forms of peptide YY are PYY₁₋₃₆ and PYY₃₋₃₆. PYY₁₋₃₆ is released postprandially from intestinal L cells in proportion to energy intake and in part truncated to PYY₃₋₃₆, which is the main circulating form of PYY and a relatively selective Y2 receptor agonist. PYY₁₋₃₆ and PYY₃₋₃₆ inhibit gastric acid secretion, gastrointestinal transit, and food intake. Food intake is inhibited both via a stimulant effect on Y2 receptors on vagal afferent neurons and an interaction with Y2 receptors in the hypothalamus, which is consistent with the ability of PYY to gain access to the brain via circumventricular organs such as the area postrema and subfornical organ. Furthermore, it is known that PYY concentrations in the blood of people with obesity are lower than those of healthy individuals. Thus, NPY2R and/or NPY4R agonists may hold potential in the treatment of obesity.

Glucagon-like peptide-1 (GLP-1) and glucose-dependent insulinotropic polypeptide (GIP), belong to the family of incretins. GLP-1 (7-37) is a 31-amino acid peptide, with the sequence HAEGTFTSDVSSYLEGQAAKEFIAWLVKGRG (SEQ ID NO: 182) and GIP is a 42-amino acid peptide, with the sequence YAEGTFISDYSIAMDKIHQQDFVNWLLAQKGKKNDWKHNITQ (SEQ ID NO: 183). GLP-1 and GIP are secreted from small intestinal L cells and K cells, respectively. GLP-1 acts via GLP-1 receptors and is known to have a sugar-dependent insulinotropic action (i.e. stimulate insulin release) and a feeding suppressive action. GIP acts via GIP receptors and is also known to have a sugar-dependent insulinotropic action, though its influence on feeding is less clear. GLP-1 receptor/GIP receptor co-agonist peptide has been reported to show a stronger hypoglycaemic action and body weight-lowering action than those of a GLP-1 receptor agonist alone. Therefore, research efforts have been made to develop GLP-1/GIP receptor co-agonists for the treatment of obesity and/or diabetes, based on the structure of natural glucagon, GIP, or GLP-1.

The most noteworthy effects of GLP-1 agonists are their ability to promote insulin secretion in a glucose-dependent manner by binding to GLP-1 receptors expressed on the pancreatic B cells. Almost as importantly, GLP-1 agonists have been shown to inhibit glucagon secretion at glucose levels above fasting levels. Critically, this does not affect the glucagon response to hypoglycaemia, making GLP-1 agonists a safe anti-diabetic drug with very low incidence of hypoglycaemia compared to insulin. In June 2021, Semaglutide, a GLP-1 agonist, was approved by the FDA for chronic weight management in adults with obesity or overweight with at least one weight-related condition (such as high blood pressure, type 2 diabetes, or high cholesterol). As an anti-obesity drug, GLP-1 agonists work by binding to GLP-1 receptors in hypothalamus thereby suppressing appetite. Furthermore, GLP-1 agonists bind to GLP-1 receptors in the stomach, inhibiting gastric emptying, acid secretion, and motility, which collectively promote satiety. Consequently, diabetic subjects treated with GLP-1 receptor agonists often also experience a beneficial weight loss in addition to a control of their blood sugar levels.

However, despite long-standing efforts, the number of overweight and obese patients is still growing. First line therapy for overweight and obese patients comprises diet and exercise but often are not sufficiently efficacious. Second line treatment options are bariatric surgery and pharmacotherapy. Available pharmacological treatments seem to lack in efficacy and/or safety, and only a limited number of approved therapies, such as Semaglutide and Tirzepatide, are available. Therefore, there is still a high medical need for more efficacious and safe treatment options. Future obesity treatments may benefit from targeting GLP1-R, GIPR and NPY2R simultaneously in view of the biological role of PYY, GIP, or GLP-1. In addition, developing mono-peptides able to target the above-mentioned three receptors are highly desirable compared to combination therapy with individual peptides for several reasons. First, mono peptides are easier to formulate into a single dosing unit as compared to mixtures of different polypeptides. This is primarily because different peptides may possess different physiochemical properties, e.g., isoelectric point, solubility, or chemical stability at a given pH, which means that one formulation developed for one peptide may be less optimal for or incompatible with a different polypeptide. Therefore, combination therapy with individual peptides may require individual dosing units.

Secondly, mono peptides are cheaper to manufacture compared to individual polypeptides and also reduce the burden of regulatory approval.

EP3467106 A1 discloses peptides that agonize receptors GLP-1, GIP and NPY2 for the treatment of obesity, diabetes, and the like. EP3467106 A1 does not report any solubility data of the disclosed peptides.

Example 41, Example 42, and Example 40 of EP3467106 A1, termed in the present application as reference compounds Ref.1, Ref.2, and Ref.3 respectively (see Table B), result to have a solubility of <1 mg/ml at or around physiological pH as showed in Table 2 of the present application. Considering the structure similarity of Example 41, 42 and 40 of EP3467106 A1 with all other peptides described in EP3467106 A1, it is credibly derivable that EP3467106 A1 peptides are no soluble at or around physiological pH.

Therefore, there is a need for novel peptide compounds that agonize receptors GLP-1, GIP and NPY2 and are soluble at or around physiological pH and therefore viable for human therapy.

Further peptides comprising forty-nine amino acids that agonize receptors GLP-1, GIP and NPY2 are disclosed in WO2024/133382 A1.

### SUMMARY OF THE INVENTION

The present invention relates to compounds of general formula (I) comprising a peptide and a fatty acid substituent.

The present invention set out to provide peptide compounds that agonize all of the receptors GLP1-R, GIPR and NPY2R (briefly, triple agonists) and that are viable for human therapy, in order to provide effective treatments for the diseases mentioned in the present specification, e.g. obesity, diabetes, metabolic syndrome, and/or MASH/NASH, by administration to a human in need thereof, preferably by administration via subcutaneous injection.

Particularly, the present invention set out to provide peptide compounds that agonize all the receptors GLP1-R, GIPR and NPY2R and are soluble at or around physiological pH for use in human therapy, preferably by administration via subcutaneous injection.

In the present contest, the term “at or around physiological pH” means at or around pH 7, e.g. pH 7±0.5.

Peptide therapeutics are usually provided as pharmaceutical liquid formulation in a ready-to-use injection device for subcutaneous administration. These peptide formulations for subcutaneous administration have limited application volumes. Therefore, good solubility of the peptides at or around physiological pH is a requirement for the application in a ready-to-use injection device.

A further aim of the present invention is to provide peptide compounds that agonize all the receptors GLP1-R, GIPR and NPY2R, are soluble at or around physiological pH, and have a long duration of action in the body (i.e. a long in-vivo half-life), in order to provide compounds suitable for the treatment of the diseases mentioned in the present specification by administration of the compound to a human in need thereof.

A further aim of the present invention is to provide peptide compounds that agonize all the receptors GLP1-R, GIPR and NPY2R, are soluble at or around physiological pH, and have a long duration of action in the body and are suitable for administration by subcutaneous injection to humans, preferably once weekly or once monthly.

A further aim of the present invention is to provide peptide compounds triple agonists that are soluble at or around physiological pH, have a long duration of action in the body, and show good selectivity for the GLP-1, GIP and NPY2 receptors against other incretin or related receptors. For example, the compounds of the invention might not activate other receptors from the NPY receptor family, such as NPY1, NPY4 or NPY5 receptor, and/or might not activate GLP-2 or glucagon receptors.

A further aim of the present invention is to provide peptide compounds triple agonists, soluble at or around physiological pH, having a long duration of action in the body, and having high chemical and physical stability in a liquid formulation suitable for subcutaneous injection to humans.

A further aim of the present invention is to provide peptide compounds triple agonists, soluble at or around physiological pH, having a long duration of action in the body suitable for a safe and tolerate treatment of humans.

In a first aspect, the invention provides compounds of general formula (I) or salt thereof, preferably a pharmaceutically acceptable salt thereof,

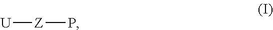

wherein P is a peptide X₁-X₃₅ and U-Z is a lipid-linker moiety as defined in the present specification.

In a second aspect, the invention relates to a pharmaceutical composition, preferably suitable for administration by subcutaneous injection, comprising at least one compound according to the first aspect together with one or more pharmaceutically acceptable carrier and/or excipients.

In a third aspect, the invention relates to compounds according to the first aspect for use as a medicament, preferably for use in a method of treatment of a disease, disorder and/or condition disclosed in the present specification.

In a fourth aspect, the invention relates to a method for the treatment of a disease, disorder and/or condition disclosed in the present specification, preferably selected from the list consisting of excessive weight, obesity, type 1 and type 2 diabetes, eating disorders, hyperlipidemia, metabolic syndrome, MAFLD/NAFLD, MASH/NASH, cardiovascular diseases, comorbidities thereof, said method comprising administering a therapeutic effective amount of a compound of the present invention according to any of the aspects and embodiments disclosed herein, to an individual in need thereof.

In a fifth aspect, the invention relates to a method for preparing compounds of the present invention according to any of the aspects and embodiments disclosed herein. The method may comprise the steps of synthesizing the compound of the invention by solid-phase or liquid-phase methodology, and optionally isolating and/or purifying the final product.

### TERMS, DEFINITIONS AND CONVENTIONS

The meanings of the residues U, Z, and P will be given hereinafter as embodiments of the invention (the ‘*’ indicate the attachment point to another moiety of the compound). Any and each of these definitions and embodiments may be combined with one another.

Terms not specifically defined herein should be given the meanings that would be given to them by one of skill in the art in light of the disclosure and the context. As used in the specification, however, unless specified to the contrary, the following terms have the meaning indicated and the following conventions are adhered to.

### Peptides (P) and Amino Acids

Peptides (P) of the present invention are peptides comprising, or alternatively, consisting of a sequence of 35 amino acids, wherein the amino acid residues are numbered from 1 to 35, i.e. X₁ to X₃₅ or X₁-X₃₅ (the number shown in subscript after an amino acid represents the amino acid position number). For example, when peptide (P) consists of 35 amino acids, the amino acid at the N-terminus is regarded as amino acid at position 1 (i.e. X₁) and the amino acid at the C-terminus is regarded as amino acid at position 35 (i.e. X₃₅). In the present contest, the position of the amino acid in peptide (P) can be indicated as subscript number (i.e. nr) if the amino acid is represented by X (i.e. Xₙᵣ), or as number in bracket after the amino acid conventional abbreviation (e.g. a lysine at position 29 can be indicated as K(29) or Lys(29)).

The term “amino acid” refers to any amino acid, naturally occurring (including the 20 standard amino acids which are encoded by the standard genetic code in humans) or not naturally occurring. According to the present invention, unless otherwise stated, the amino acids are all L-amino acids (L-stereoisomer, natural amino acids).

In the present specification, unless naturally occurring amino acids are referred to by their full name (e.g. alanine, arginine, etc.), they are designated by their conventional three-letter or single-letter abbreviations (e.g. Arg or R for arginine, etc.). In the case of less common or non-naturally occurring amino acids, unless they are referred to by their full name, conventional abbreviations are employed according to the following table I or table III (the ‘*’ indicate the attachment point to another moiety of the compound). A hyphen in a term listed in table I and III may be present or omitted and in both case the term represents the same meaning.

In the present context, substitution of one or more amino acid in peptide (P) may be substitution with natural amino acids as well as unnatural amino acids, including L- and D-stereoisomers thereof.

TABLE I

| abbreviation | chemical name | formula |
|---|---|---|
| Aib | refers to the residue of aminoisobutyric acid (IUPAC name: 2-amino-2-methylpropanoic acid) | 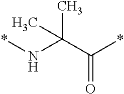 [SMILES: *NC(C)(C)C(*)=O] |
| NMeQ | refers to the residue of N-methyl-L-glutamine, (IUPAC name: (2S)-2-(methylamino)pentanedioic acid) | 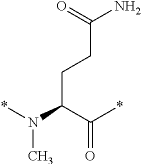 [SMILES: *C(=O)[C@H](CCC(N)=O)N(*)C] |
| NMeR | refers to the residue of N-methyl-L-arginine (IUPAC name: (2S)-5- (diaminomethylideneamino)-2- (methylamino)pentanoic acid) | 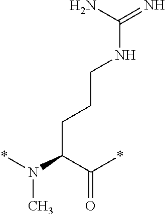 [SMILES: *C(=O)[C@H](CCCNC(=N)N)N(*)C] |
| NMeY | refers to the residue of N-methyl-L-tyrosine (IUPAC name: (2S)-3-(4-hydroxyphenyl)-2- (methylamino)propionic acid) | 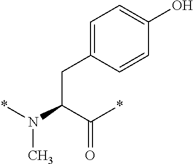 [SMILES: *C(=O)[C@H](Cc1ccc(O)cc1)N(*)C] |
| ArgMe | refers to the residue of omega-methyl-L-arginine (IUPAC name: (2S)-2-amino-5-[(N′- methylcarbamimidoyl)amino]pentanoate) | 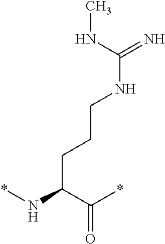 [SMILES: *N[C@@H](CCCNC(=N)NC)C(*)=O] |
| bhArg | refers to the residue of beta-homo-L-arginine (IUPAC name: (3S)-3-amino-6- (diaminomethylideneamino)hexanoic acid) | 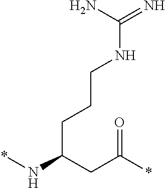 [SMILES: *N[C@@H](CCCNC(=N)N)CC(*)=O] |
| dOR | refers to the residue of de-oxo-arginine (synonym: N-[(4R)-4-aminopentyl]guanidine) | 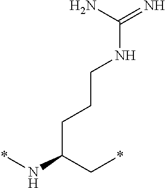 [SMILES: *C[C@H](CCCNC(=N)N)N*] |
| Phe4F | refers to the residue of 4-fluoro-L-phenylalanine (IUPAC name: (2S)-2-amino-3-(4- fluorophenyl)propanoic acid) | 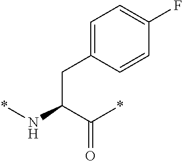 [SMILES: *N[C@@H](Cc1ccc(F)cc1)C(*)=O] |

### Lipid (U) and Linker (Z) Moieties

The compounds of the invention of formula (I) U-Z-P comprise a peptide (P), a lipid moiety (U) and a linker moiety (Z), wherein the lipid (U) is covalently attached to the linker (Z) in one end and the peptide (P) is covalently attached to the linker (Z) in the opposite end (i.e. lipid-linker-peptide, U-Z-P), wherein the linker (Z) is bonded to the epsilon-amino group of Lys at position-29 (Lys(29)) of the peptide (P).

Without wishing to be bound by any theory, it is thought that such lipophilic substituents bind albumin and other plasma components in the blood stream, thereby shielding the compound of the invention from renal filtration as well as enzymatic degradation. Thus, lipidation of a peptide is typically performed to improve the pharmacokinetic profile of a peptide by e.g. improving metabolic stability, reducing enzymatic degradation, lowering excretion and metabolism, all in all resulting in a prolonged in vivo half-life (t_{1/2}).

In addition, in the present contest it is thought that the lipid-linker moieties (U-Z) contribute to provide to compounds of formula (I) a suitable solubility at or around physiological pH for use in human therapy, preferably by administration via subcutaneous injection. In particular, the compound of general formula (I) comprising at least six (6) residues of amino acids selected from the group consisting of E and [gGlu] showed suitable solubility at or around physiological pH for use in human therapy, preferably by administration via subcutaneous injection.

The lipid (U) is a hydrocarbon chain, may be linear or branched, and may be saturated or unsaturated. Furthermore, it may include a functional group at the end of the hydrocarbon chain, e.g. a carboxylic acid group, a sulphonic acid group, or a tetrazole group.

In a preferred embodiment, lipid (U) is according to formula (II)

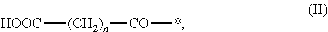

wherein n is an integer in the range 12-20. Formula (II) may also be referred to as a C(n+2) diacid (briefly, C(n+2) DA), wherein n is an integer in the range 12-20, preferably n is 14, 16, or 18 (briefly, C16DA, C18DA, and C20DA respectively), illustrated in table II.

TABLE II

| abbreviation | formula or chemical name | formula |
|---|---|---|
| C(n + 2)DA | HOOC—(CH₂)n-CO—* (formula (II)) | 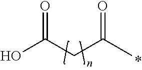 |
| C16DA | 15-carboxy-pentadecanoyl | 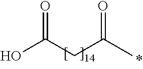 |
| C18DA | 17-carboxy-heptadecanoyl | 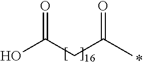 |
| C20DA | 19-carboxy-nonadecanoyl | 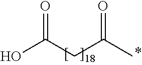 |

The linker (Z) consists of three (3) to ten (10) linker sub-moieties termed Z¹, Z², Z³, Z⁴, Z⁵, Z⁶, Z⁷, Z⁸, Z⁹, and Z¹⁰ covalently connected each other, wherein the linker sub-moieties are independently selected from monomer commonly used in the art or absent (i.e. *-Z¹-Z²-Z³-*; *-Z¹-Z²-Z³-Z⁴-*; *-Z¹-Z²-Z³-Z⁴-Z⁵-*; *-Z¹-Z²-Z³-Z⁴-Z⁵-Z⁶*; *-Z¹-Z²-Z³-Z⁴-Z⁵-Z⁶-Z⁷-*; *-Z¹-Z²-Z³-Z⁴-Z⁵-Z⁶-Z⁷-Z⁸-*; *-Z¹-Z²-Z³-Z⁴-Z⁵-Z⁶-Z⁷-Z⁸-Z⁹-*; or *-Z¹-Z²-Z³-Z⁴-Z⁵-Z⁶-Z⁷-Z⁸-Z⁹-Z¹⁰-*). In a preferred embodiment the linker (Z) consists of three (3) to eight (8) linker sub-moieties.

In a preferred embodiment, linker (Z) consists of three (3) to ten (10) linker sub-moieties termed Z¹, Z², Z³, Z⁴, Z⁵, Z⁶, Z⁷, Z⁸, Z⁹, and Z¹⁰ covalently connected each other, wherein the linker sub-moieties are independently selected from the group consisting of: absent, [gGlu], [OEG], [eLys], [AHX], [TRX], [gDab], and [dtOrn], wherein at least one linker sub-moiety is [gGlu], and wherein the first linker sub-moieties is linked to lipid (U) and the last linker sub-moiety on the other end of the linker is linked to the epsilon-amino group of Lys at position-29 [Lys(29)] of the peptide (P) (i.e. U-Z¹-Z²-Z³-Z⁴-Z⁵-Z⁶-Z⁷-Z⁸-Z⁹-Z¹⁰-P). In an even more preferred embodiment, the linker (Z) consists of three (3) to eight (8) linker sub-moieties.

The meaning of linker sub-moieties abbreviations is illustrated in table III; the ‘*’ indicate the attachment point to another moiety of the compound; the “(Rₙ)” beside the “*” represents the R-group number used; the bracket (i.e. [ . . . ]) in a term listed in table III may be present or omitted and in both case the term represents the same meaning. The “*” represented in table III correspond to the R-group number of the Boehringer Ingelheim Line Notation (BILN) used for the nomenclature of the compounds of the invention, as described hereinafter, wherein the “*” connected to the carboxy-group corresponds to the R“2”-group of the BILN and the “*” connected to the amino-group corresponds to the R“1”-group of the BILN.

TABLE III

| abbreviation | chemical name | formula |
|---|---|---|
| [gGlu] | refers to the residue of gamma-L-glutamate (IUPAC name: (2S)-2-aminopentanedioic acid) | 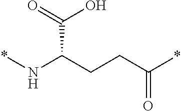 [SMILES: *N[C@@H](CCC(*)=O)C(=O)O] |
| [eLys] | refers to the residue of epsilon-L-lysine (IUPAC name: (2S)-2,6-diaminohexanoic acid) | 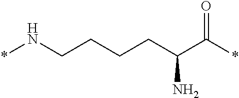 [SMILES: *NCCCC[C@H](N)C(*)=O] |
| [AHX] | refers to the residue of 6-aminohexanoic acid (IUPAC name: 6-aminohexanoic acid) | 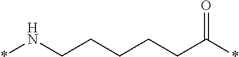 [SMILES: *NCCCCCC(*)=O] |
| [OEG] | refers to the residue of 8-amino-3,6- dioxaoctanoic acid (IUPAC name: 2-[2-(2- aminoethoxy)ethoxy]acetic acid) | 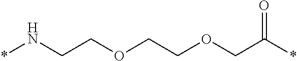 [SMILES: *NCCOCCOCC(*)=O] |
| [TRX] | refers to the residue of tranexamic acid (IUPAC name: 4-(aminomethyl)cyclohexane-1- carboxylic acid) | 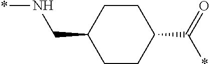 [SMILES: *NC[C@H]1CC[C@H](C(*)=O)CC1] |
| [gDab] | refers to the residue of gamma-L-diaminobutyric acid (IUPAC name: 2,4-diaminobutanoic acid; synonym: (2S)-2,4-diaminobutyric acid, or Dbu) | 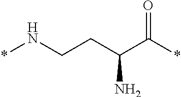 [SMILES: *NCC[C@H](N)C(*)=O] |
| [dtOrn] | refers to the residue of delta-L-ornithine, (IUPAC name: (2S)-2,5-diaminopentanoic acid) | 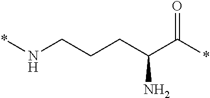 [SMILES: *NCCC[C@H](N)C(*)=O] |

As an example, a lipid-linker moiety (U-Z) represented by HOOC—(CH₂)ₙ—CO-Z¹-Z²-Z³-Z⁴-Z⁵-Z⁶-Z⁷-Z⁸-Z⁹-Z¹⁰-* is bonded to the epsilon-amino group of Lys at position-29 [Lys(29)], wherein linker sub-moiety Z¹ is bonded to the carboxy group of lipid (U) and linker sub-moiety Z¹⁰ is bonded to the epsilon-amino group of Lys(29), to assume the following structure:

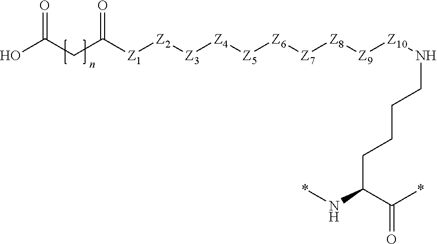

### N- and C-terminal of Peptide P

In the present context, peptides (P) are generally amidated at the C-terminal (—CONH₂), like the native peptides. However, the peptides (P) of the present invention may also have either a free carboxylic acid (—COOH) or another post-translational modification, such as a methyl ester (—COOMe). In a preferred embodiment of the invention, the peptides are amidated at the C-terminal (P-NH₂, i.e. U-L-P-NH₂).

The peptides (P) according to the present invention may have a free amine (—NH₂), be N-acylated (—NHCOR), N-methylated (—NHCH₃ or —N(CH₃)₂), deaminated at the N-terminal, or N-lipidated. In a preferred embodiment of the invention, the peptides have a free amine at the N-terminal.

In a preferred embodiment of the invention, the peptides are amidated at the C-terminal (P-NH₂) and have a free amine at the N-terminal.

### Nomenclature of Compounds:

Compounds of the present invention are represented in the Boehringer Ingelheim Line Notation (BILN), which describes complex peptides in a human-readable format (Fox et al, J. Chem. Inf. Model. 2022, 62, 17, 3942-3947,). In BILN, chains are built up of monomers, e.g. A represents alanine, Aib represents isobutyric acid, and two monomers are linked by a hyphen “-”. Different chains are separated by a dot “.” and connections between chains are explicitly defined in brackets following the monomer, which includes the link ID number and the R-group number, e.g. K(1,3) represents a lysine residue that is linked to another monomer carrying the same link ID “1” by its R“3”-group (i.e. epsilon-amino group). As an example, Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2·C20DA-gGlu-gGlu-gGlu-gGlu-gGlu-AHX-AHX (1,2) (SEQ ID NO: 1), wherein C20DA represents 19-carboxynonadecanoyl, gGlu represents gamma-L-glutamate connected via its amino-group to C20DA and via its gamma-carboxy-group to the amino-group of the next gGlu, which is repeated four times resulting in five gGlu residues, connected via the final gamma-carboxy-group to the amino-group of 6-aminohexanoic acid (AHX), which is repeated one more time and connected via the final carboxy-group (i.e. AHX, R“2”-group) to the epsilon-amino-group (i.e. K, R“3”-group) of lysine (K) in position 29 of the peptide backbone, completely defines the following structure:

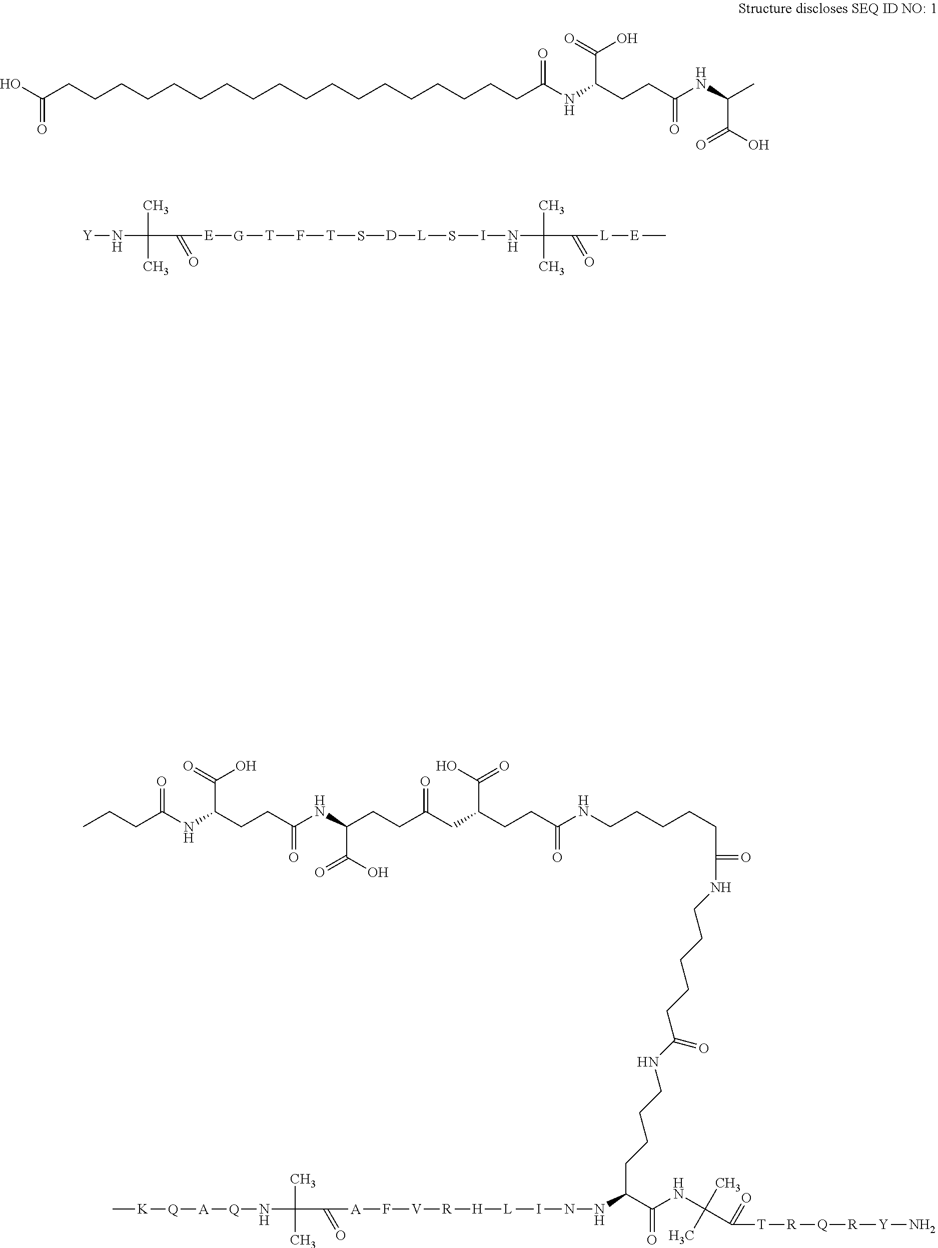

For clarity, as disclosed in the above-mentioned J. Chem. Inf. Model. 2022, 62, 17, 3942-3947, with respect to lysin residue at position 29 (Lys(29)) in the compounds of the invention, in the BILN used in the present specification the R-group number connected to the alpha-carboxy-group is termed R“2”-group, the R-group number connected to the alpha-amino-group is termed R“1”-group, and the R-group number connected to the epsilon-amino-group is termed R“3”-group.

### Pharmaceutically Acceptable Salts

According to the present invention, the compound of the invention may be in the form of a pharmaceutically acceptable salt. Pharmaceutically acceptable salts are intended to include any salts that are commonly used in formulations of peptides or derivative thereof. Such salts include both acid addition salts and basic salts, and examples may be found in e.g., Remington's pharmaceutical sciences, 17^{th} edition and more recent editions thereof.

### Additional Definitions

The term “agonist” as employed in the context of the invention refers to a substance that activates the receptor type in question, typically by binding to it (i.e. as a ligand).

The terms “treatment” and grammatical variants thereof (e.g. “treated”, “treating”, “treat”) as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject in need of treatment may thus be a subject already afflicted with the disease or disorder in question. The term “treatment” includes inhibition or reduction of an increase in severity of a pathological state or symptoms (e.g. weight gain or hyperglycemia) relative to the absence of treatment, and is not necessarily meant to imply complete cessation of the relevant disease, disorder or condition.

The terms “prevention” and grammatical variants thereof (e.g., “prevented”, “preventing”, “prevent”) as employed in the present context refer to an approach for hindering or preventing the development of, or altering the pathology of, a condition, disease, or disorder. Accordingly, “prevention” may refer to prophylactic or preventive measures. For the purposes of this invention, beneficial or desired clinical results include, but are not limited to, prevention or slowing of symptoms, progression, or development of a disease, whether detectable or undetectable. A subject in need of “prevention” may thus be a subject not yet afflicted with the disease or disorder in question. The term “prevention” thus includes inhibiting or slowing the onset of disease relative to the absence of treatment and is not necessarily meant to imply permanent prevention of the relevant disease, disorder, or condition.

The terms “patient”, “subject,” and “individual” may be used interchangeably and refer to a human.

### DETAILED DESCRIPTION OF THE INVENTION

In a first aspect, the invention provides a compound according to general formula (I)

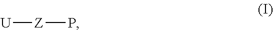

wherein P is a peptide comprising or, alternatively, consisting of the amino acid sequence,

(SEQ ID NO: 184)
Y-Aib-E-X₄-X₅-F-T-S-D-X₁₀-X₁₁-X₁₂-X₁₃-L-X₁₅-X₁₆-X₁₇-_{X18}-X₁₉-X₂₀-X₂₁-F-X₂₃-R-X₂₅-
X₂₆-X₂₇-X₂₈-K-X₃₀-T-R-X₃₃-X₃₄-X₃₅

wherein
X₄ is selected from the group consisting of A, and G,
X₅ is selected from the group consisting of A, E, and T,
X₁₀ is selected from the group consisting of F, L, and Y,
X₁₁ is selected from the group consisting of A, and S,
X₁₂ is selected from the group consisting of A, E, and I,
X₁₃ is selected from the group consisting of A, Aib, E, L, and aMeL,
X₁₅ is selected from the group consisting of D, and E,
X₁₆ is selected from the group consisting of A, Aib, E, G, K, L, LysAc, N, Q, R,
X₁₇ is selected from the group consisting of A, E, I, and Q,
X₁₈ is selected from the group consisting of A, E and R,
X₁₉ is selected from the group consisting of A, E, Q, and Y,
X₂₀ is selected from the group consisting of A, Aib, E, and Y,
X₂₁ is selected from the group consisting of A, E, and S,
X₂₃ is selected from the group consisting of I, L, and V,
X₂₅ is selected from the group consisting of A, H, Q, and Y,
X₂₆ is selected from the group consisting of A, and L,
X₂₇ is selected from the group consisting of I, L, and Y,
X₂₈ is selected from the group consisting of A, and N,
X₃₀ is selected from the group consisting of A, Aib, I, L, V, and W,
X₃₃ is selected from the group consisting of NMeQ, and Q,
X₃₄ is selected from the group consisting of dOR, R, NMeR, MeR (ArgMe), bhR(bhArg), preferred dOR, R,
X₃₅ is selected from the group consisting of Phe4F, Y, and NMeY;
wherein U is lipid moiety comprising a hydrocarbon chain;
wherein Z is a linker moiety comprising at least one [gGlu] residue;
wherein Z is linked to U in one end and to the epsilon-amino group of Lys at position-29 of the peptide P in the other end;
wherein compound of general formula (I) comprises at least six (6) residues of amino acids selected from the group consisting of E and [gGlu], such as in the range 6 to 10 (i.e. 6, 7, 8, 9, 10);
   or salt thereof, preferably a pharmaceutically acceptable salt thereof.

For clarity, in general formula (I), in any embodiment thereof, and in any compound according to general formula (I) disclosed in the present specification the carboxy-group of the residue X₂₈ is linked to the alpha-amino group of Lys residue at position-29 of the peptide P.

### Examples of Compounds of the Invention

TABLE A

| | | SEQ |
|---|---|---|
| Compound | | ID |
| No | U-Z-P | NO |
| 1 | Y-Aib-E-G-T-F-T-S-D-L-S-I-Aib-L-E-K-Q-A-Q-Aib-A-F-V-R-H-L-I-N-K(1,3)-Aib-T- | 1 |
| | R-Q-R-Y-NH2.C20DA-gGlu-gGlu-gGlu-gGlu-gGlu-AHX-AHX(1,2) | |
| 2 | Y-Aib-E-G-T-F-T-S-D-Y-S-I-Aib-L-E-K-Q-A-Q-Aib-A-F-V-R-H-L-I-N-K(1,3)-Aib-T- | 2 |
| | R-NMeQ-R-Y-NH2.C18DA-gGlu-gGlu-gGlu-gGlu-gGlu-gGlu(1,2) | |
| 3 | Y-Aib-E-G-T-F-T-S-D-Y-S-I-Aib-L-E-K-Q-A-Q-Aib-A-F-V-R-H-L-I-N-K(1,3)-Aib-T- | 3 |
| | R-Q-R-Y-NH2.C20DA-gGlu-gGlu-gGlu-gGlu-gGlu-gGlu(1,2) | |
| 4 | Y-Aib-E-G-T-F-T-S-D-Y-S-I-Aib-L-E-K-A-A-Q-Aib-A-F-V-R-H-L-I-N-K(1,3)-Aib-T- | 4 |
| | R-Q-R-Y-NH2.C20DA-gGlu-gGlu-gGlu-gGlu-gGlu-gGlu-eLys(1,2) | |
| 5 | Y-Aib-E-G-T-F-T-S-D-Y-S-I-Aib-L-E-Aib-Q-A-Q-Aib-A-F-V-R-H-L-I-N-K(1,3)-Aib- | 5 |
| | T-R-Q-R-Y-NH2.C18DA-gGlu-gGlu-gGlu-gGlu-gGlu-gGlu-eLys(1,2) | |
| 6 | Y-Aib-E-G-T-F-T-S-D-Y-S-I-Aib-L-E-N-Q-A-Q-Aib-A-F-V-R-H-L-I-N-K(1,3)-Aib-T- | 6 |
| | R-Q-R-Y-NH2.C18DA-gGlu-gGlu-gGlu-gGlu-gGlu-gGlu-eLys(1,2) | |
| 7 | Y-Aib-E-G-T-F-T-S-D-Y-S-I-Aib-L-E-K-Q-A-Q-Aib-A-F-V-R-H-L-I-N-K(1,3)-L-T-R- | 7 |
| | Q-R-Y-NH2.C18DA-gGlu-gGlu-gGlu-gGlu-gGlu-OEG(1,2) | |
| 8 | Y-Aib-E-G-T-F-T-S-D-Y-S-I-Aib-L-E-K-Q-A-Q-Aib-E-F-V-R-H-L-I-N-K(1,3)-Aib-T- | 8 |
| | R-Q-R-Y-NH2.C18DA-gGlu-gGlu-gGlu-gGlu-gGlu-OEG(1,2) | |
| 9 | Y-Aib-E-G-T-F-T-S-D-Y-S-I-Aib-L-E-K-Q-A-Q-Aib-A-F-V-R-H-L-I-N-K(1,3)-Aib-T- | 9 |
| | R-Q-R-Y-NH2.C18DA-gGlu-gGlu-gGlu-gGlu-gGlu-gGlu-AHX(1,2) | |
| 10 | Y-Aib-E-G-T-F-T-S-D-Y-S-I-Aib-L-E-K-Q-A-E-Aib-A-F-V-R-H-L-I-N-K(1,3)-Aib-T- | 10 |
| | R-Q-R-Y-NH2.C18DA-gGlu-gGlu-gGlu-gGlu-glu-gGlu-eLys(1,2) | |
| 11 | Y-Aib-E-G-T-F-T-S-D-Y-S-I-Aib-L-E-K-Q-A-Q-Aib-A-F-V-R-H-L-I-N-K(1,3)-Aib-T- | 11 |
| | R-Q-R-Y-NH2.C18DA-gGlu-gGlu-gGlu-gGlu-gGlu-gGlu-eLys-eLys(1,2) | |
| 12 | Y-Aib-E-G-T-F-T-S-D-Y-S-I-Aib-L-E-K-Q-A-Q-Aib-A-F-V-R-H-L-I-N-K(1,3)-L-T-R- | 12 |
| | Q-R-Y-NH2.C18DA-gGlu-gGlu-gGlu-gGlu-gGlu-gGlu(1,2) | |
| 13 | Y-Aib-E-G-T-F-T-S-D-Y-S-I-Aib-L-E-K-Q-A-Q-Aib-A-F-V-R-H-L-I-N-K(1,3)-Aib-T- | 13 |
| | R-Q-R-Y-NH2.C18DA-gGlu-gGlu-gGlu-gGlu-gGlu-AHX(1,2) | |
| 14 | Y-Aib-E-G-T-F-T-S-D-Y-S-I-Aib-L-E-K-Q-A-Q-Aib-E-F-V-R-H-L-I-N-K(1,3)-Aib-T- | 14 |
| | R-Q-R-Y-NH2.C18DA-gGlu-gGlu-gGlu-gGlu-gGlu-AHX(1,2) | |
| 15 | Y-Aib-E-G-T-F-T-S-D-F-S-I-Aib-L-E-K-Q-A-Q-Aib-A-F-V-R-H-L-I-N-K(1,3)-Aib-T- | 15 |
| | R-Q-R-Y-NH2.C20DA-gGlu-gGlu-gGlu-gGlu-Glu-gGlu-eLys(1,2) | |
| 16 | Y-Aib-E-G-T-F-T-S-D-Y-S-I-Aib-L-D-K-Q-A-Q-Aib-A-F-V-R-H-L-I-N-K(1,3)-Aib-T- | 16 |
| | R-Q-dOR-Y-NH2.C20DA-gGlu-gGlu-gGlu-gGlu-gGlu-gGlu-gGlu-eLys(1,2) | |
| 17 | Y-Aib-E-G-T-F-T-S-D-Y-S-I-Aib-L-E-K-Q-A-Q-Aib-A-F-V-R-H-L-I-N-K(1,3)-Aib-T-R- | 17 |
| | NMeQ-R-Y-NH2.C18DA-gGlu-gGlu-gGlu-gGlu-gGlu-gGlu-eLys(1,2) | |
| 18 | Y-Aib-E-G-T-F-T-S-D-Y-S-I-Aib-L-E-K-Q-A-Q-Aib-A-F-V-R-H-L-I-N-K(1,3)-I-T-R- | 18 |
| | Q-R-Y-NH2.C18DA-gGlu-gGlu-gGlu-gGlu-gGlu-gGlu-Glu-eLys(1,2) | |
| 19 | Y-Aib-E-G-T-F-T-S-D-Y-S-I-Aib-L-E-K-Q-A-Q-Aib-E-F-V-R-H-L-I-N-K(1,3)-Aib-T- | 19 |
| | R-Q-R-Y-NH2.C18DA-gGlu-gGlu-gGlu-gGlu-gGlu-OEG(1,2) | |
| 20 | Y-Aib-E-G-T-F-T-S-D-L-S-I-Aib-L-E-K-Q-A-Q-Aib-A-F-V-R-H-L-I-N-K(1,3)-Aib-T- | 20 |
| | R-Q-R-Y-NH2.C20DA-gGlu-gGlu-gGlu-Glu-gGlu-gGlu-eLys(1,2) | |
| 21 | Y-Aib-E-G-T-F-T-S-D-L-S-I-Aib-L-E-K-Q-A-Q-Aib-A-F-I-R-H-L-I-N-K(1,3)-Aib-T- | 21 |
| | R-Q-R-Y-NH2.C20DA-gGlu-gGlu-gGlu-gGlu-gGlu-gGlu-eLys(1,2) | |
| 22 | Y-Aib-E-G-T-F-T-S-D-Y-S-I-aMeL-L-D-K-Q-A-Q-Aib-A-F-I-R-H-L-I-N-K(1,3)-Aib- | 22 |
| | T-R-Q-R-Y-NH2.C20DA-gGlu-gGlu-gGlu-gGlu-Glu-gGlu-eLys(1,2) | |
| 23 | Y-Aib-E-G-T-F-T-S-D-Y-S-I-Aib-L-E-Q-Q-A-Q-Aib-A-F-V-R-H-L-I-N-K(1,3)-Aib-T- | 23 |
| | R-Q-R-Y-NH2.C18DA-gGlu-gGlu-gGlu-Glu-gGlu-gGlu-eLys(1,2) | |
| 24 | Y-Aib-E-G-T-F-T-S-D-Y-S-I-Aib-L-E-K-I-A-Q-Aib-A-F-V-R-H-L-I-N-K(1,3)-Aib-T- | 24 |
| | R-Q-R-Y-NH2.C18DA-gGlu-gGlu-gGlu-gGlu-gGlu-Glu-eLys(1,2) | |
| 25 | Y-Aib-E-G-T-F-T-S-D-Y-S-I-Aib-L-E-E-E-A-Q-Aib-A-F-V-R-H-L-I-N-K(1,3)-Aib-T- | 25 |
| | R-Q-R-Y-NH2.C18DA-gGlu-gGlu-gGlu-gGlu(1,2) | |
| 26 | Y-Aib-E-G-T-F-T-S-D-Y-S-I-Aib-L-E-K-Q-A-Q-Aib-A-F-V-R-H-L-I-N-K(1,3)-Aib-T- | 26 |
| | R-NMeQ-R-Y-NH2.C20DA-gGlu-gGlu-gGlu-gGlu-gGlu-Glu-eLys(1,2) | |
| 27 | Y-Aib-E-G-T-F-T-S-D-Y-S-I-Aib-L-E-K-Q-A-Q-Aib-A-F-V-R-H-L-I-N-K(1,3)-L-T-R- | 27 |
| | Q-R-Y-NH2.C20DA-gGlu-gGlu-gGlu-gGlu-gGlu-gGlu-eLys(1,2) | |
| 28 | Y-Aib-E-G-T-F-T-S-D-Y-S-I-Aib-L-E-K-E-A-Q-Aib-A-F-V-R-H-L-I-N-K(1,3)-Aib-T- | 28 |
| | R-Q-R-Y-NH2.C20DA-gGlu-gGlu-gGlu-gGlu-gGlu-gGlu-eLys(1,2) | |
| 29 | Y-Aib-E-G-T-F-T-S-D-Y-S-I-A-L-E-K-Q-A-Q-Aib-A-F-V-R-H-L-I-N-K(1,3)-Aib-T-R- | 29 |
| | Q-R-Y-NH2.C20DA-gGlu-gGlu-gGlu-gGlu-gGlu-gGlu(1,2) | |
| 30 | Y-Aib-E-G-T-F-T-S-D-Y-S-I-Aib-L-E-K-Q-A-Q-Aib-E-F-V-R-H-L-I-N-K(1,3)-Aib-T- | 30 |
| | R-Q-R-Y-NH2.C18DA-gGlu-gGlu-gGlu-gGlu-gGlu-gGlu-gGlu-eLys(1,2) | |
| 31 | Y-Aib-E-G-T-F-T-S-D-Y-S-I-Aib-L-E-K-Q-A-Q-Aib-A-F-V-R-H-L-I-N-K(1,3)-Aib-T- | 31 |
| | R-Q-R-Y-NH2.C18DA-TRX-gGlu-gGlu-gGlu-gGlu-gGlu-gGlu(1,2) | |
| 32 | Y-Aib-E-G-T-F-T-S-D-Y-S-I-Aib-L-E-K-Q-A-Q-Aib-E-F-V-R-H-L-I-N-K(1,3)-L-T-R- | 32 |
| | Q-R-Y-NH2.C18DA-gGlu-gGlu-gGlu-gGlu-gGlu-gGlu-Glu-eLys(1,2) | |
| 33 | Y-Aib-E-G-T-F-T-S-D-Y-S-I-Aib-L-D-K-I-A-Q-Aib-A-F-V-R-H-L-I-N-K(1,3)-Aib-T- | 33 |
| | R-Q-R-Y-NH2.C18DA-gGlu-gGlu-gGlu-gGlu-gGlu-gGlu-eLys(1,2) | |
| 34 | Y-Aib-E-G-T-F-T-S-D-Y-S-I-Aib-L-E-K-Q-A-Q-Aib-A-F-V-R-H-L-I-N-K(1,3)-Aib-T- | 34 |
| | R-Q-R-Y-NH2.C18DA-gGlu-gGlu-gGlu-gGlu-eLys-gGlu-gGlu(1,2) | |
| 35 | Y-Aib-E-G-T-F-T-S-D-Y-S-I-Aib-L-E-K-Q-A-Q-Aib-A-F-V-R-H-L-I-N-K(1,3)-Aib-T- | 35 |
| | R-Q-R-Y-NH2.C20DA-gGlu-gGlu-gGlu-Glu-gGlu-gGlu-gGlu-eLys(1,2) | |
| 36 | Y-Aib-E-G-T-F-T-S-D-Y-S-I-Aib-L-E-K-Q-E-Q-Aib-A-F-V-R-H-L-I-N-K(1,3)-Aib-T- | 36 |
| | R-Q-R-Y-NH2.C18DA-gGlu-gGlu-gGlu-gGlu-gGlu-gGlu-eLys(1,2) | |
| 37 | Y-Aib-E-G-T-F-T-S-D-Y-S-I-Aib-L-E-K-Q-A-Q-Aib-A-F-L-R-H-L-I-N-K(1,3)-Aib-T- | 37 |
| | R-Q-R-Y-NH2.C18DA-gGlu-gGlu-gGlu-gGlu-gGlu-gGlu-eLys(1,2) | |
| 38 | Y-Aib-E-G-T-F-T-S-D-Y-S-I-Aib-L-E-K-Q-A-Q-Aib-A-F-V-R-H-L-I-N-K(1,3)-Aib-T- | 38 |
| | R-Q-R-Y-NH2.C18DA-gGlu-gGlu-gGlu-gGlu-gGlu-gGlu-gGlu(1,2) | |
| 39 | Y-Aib-E-G-T-F-T-S-D-Y-S-I-Aib-L-E-K-Q-A-Q-Aib-E-F-V-R-H-L-I-N-K(1,3)-L-T-R- | 39 |
| | Q-R-Y-NH2.C20DA-gGlu-gGlu-gGlu-gGlu-gGlu-gGlu-eLys(1,2) | |
| 40 | Y-Aib-E-G-T-F-T-S-D-Y-S-I-Aib-L-D-E-Q-A-Q-Aib-A-F-V-R-H-L-I-N-K(1,3)-Aib-T- | 40 |
| | R-Q-R-Y-NH2.C18DA-gGlu-gGlu-gGlu-gGlu-gGlu-gGlu-eLys(1,2) | |
| 41 | Y-Aib-E-G-T-F-T-S-D-Y-S-I-Aib-L-E-K-Q-A-Q-Aib-A-F-V-R-H-L-I-N-K(1,3)-I-T-R- | 41 |
| | Q-R-Y-NH2.C18DA-gGlu-gGlu-gGlu-gGlu-gGlu(1,2) | |
| 42 | Y-Aib-E-G-T-F-T-S-D-Y-S-I-Aib-L-E-Q-Q-A-Q-Aib-A-F-V-R-H-L-I-N-K(1,3)-Aib-T- | 42 |
| | R-Q-R-Y-NH2.C18DA-gGlu-gGlu-gGlu-gGlu-gGlu(1,2) | |
| 43 | Y-Aib-E-G-T-F-T-S-D-Y-S-I-Aib-L-E-K-Q-A-Q-Aib-A-F-V-R-H-L-I-N-K(1,3)-Aib-T- | 43 |
| | R-Q-NMeR-Y-NH2.C18DA-gGlu-gGlu-gGlu-gGlu-gGlu-gGlu-eLys(1,2) | |
| 44 | Y-Aib-E-G-T-F-T-S-D-Y-S-I-Aib-L-E-K-Q-A-Q-Aib-A-F-V-R-H-L-I-N-K(1,3)-Aib-T- | 44 |
| | R-Q-R-Y-NH2.C18DA-gGlu-gGlu-gGlu-gGlu-gGlu-AHX-AHX(1,2) | |
| 45 | Y-Aib-E-G-T-F-T-S-D-Y-S-I-Aib-L-E-K-Q-A-Q-Aib-E-F-V-R-H-L-I-N-K(1,3)-L-T-R- | 45 |
| | Q-R-Y-NH2.C18DA-gGlu-gGlu-gGlu-gGlu-gGlu-AHX(1,2) | |
| 46 | Y-Aib-E-G-T-F-T-S-D-Y-S-I-Aib-L-E-K-Q-A-Q-Aib-A-F-V-R-H-L-I-N-K(1,3)-Aib-T- | 46 |
| | NR-Q-R-Y-H2.C20DA-gGlu-gGlu-gGlu-gGlu-gGlu-gGlu-gDab(1,2) | |
| 47 | Y-Aib-E-G-T-F-T-S-D-Y-S-I-Aib-L-E-K-Q-A-Q-Aib-A-F-V-R-H-L-I-N-K(1,3)-Aib-T- | 47 |
| | R-Q-R-Y-NH2.C18DA-gGlu-gGlu-gGlu-gGlu-gGlu-gGlu-OEG(1,2) | |
| 48 | Y-Aib-E-G-T-F-T-S-D-Y-S-I-Aib-L-E-E-Q-A-Q-Aib-A-F-V-R-H-L-I-N-K(1,3)-Aib-T- | 48 |
| | R-Q-R-Y-NH2.C18DA-gGlu-gGlu-gGlu-gGlu-gGlu-eLys(1,2) | |
| 49 | Y-Aib-E-G-T-F-T-S-D-Y-S-I-aMeL-L-E-K-Q-A-Q-Aib-A-F-V-R-H-L-I-N-K(1,3)-Aib- | 49 |
| | T-R-Q-R-Y-NH2.C20DA-gGlu-gGlu-gGlu-gGlu-gGlu-gGlu(1,2) | |
| 50 | Y-Aib-E-G-T-F-T-S-D-Y-S-I-Aib-L-E-K-Q-A-Q-Aib-A-F-V-R-Q-L-I-N-K(1,3)-Aib-T- | 50 |
| | R-Q-R-Y-NH2.C18DA-gGlu-gGlu-gGlu-gGlu-gGlu(1,2) | |
| 51 | Y-Aib-E-G-T-F-T-S-D-L-S-I-Aib-L-E-K-Q-A-Q-Aib-A-F-V-R-H-L-I-N-K(1,3)-Aib-T- | 51 |
| | R-Q-R-Y-NH2.C20DA-gGlu-gGlu-gGlu-gGlu-gGlu-gGlu(1,2) | |
| 52 | Y-Aib-E-G-T-F-T-S-D-Y-S-I-Aib-L-E-K-Q-A-Q-Aib-A-F-V-R-H-L-I-N-K(1,3)-Aib-T- | 52 |
| | R-Q-R-Y-NH2.C20DA-gGlu-gGlu-gGlu-Glu-eLys-gGlu-gGlu(1,2) | |
| 53 | Y-Aib-E-G-T-F-T-S-D-Y-S-I-aMeL-L-E-K-Q-A-Q-Aib-A-F-I-R-H-L-I-N-K(1,3)-Aib- | 53 |
| | T-R-Q-R-Y-NH2.C20DA-gGlu-gGlu-gGlu-gGlu-gGlu-gGlu-eLys(1,2) | |
| 54 | Y-Aib-E-G-E-F-T-S-D-Y-S-I-Aib-L-E-K-Q-A-Q-Aib-A-F-V-R-H-L-I-N-K(1,3)-Aib-T- | 54 |
| | R-Q-R-Y-NH2.C18DA-gGlu-gGlu-gGlu-gGlu-gGlu-gGlu-eLys(1,2) | |
| 55 | Y-Aib-E-G-T-F-T-S-D-Y-S-I-Aib-L-D-A-Q-A-Q-Aib-A-F-V-R-H-L-I-N-K(1,3)-Aib-T- | 55 |
| | R-Q-R-Y-NH2.C18DA-gGlu-gGlu-gGlu-gGlu-gGlu-gGlu-eLys(1,2) | |
| 56 | Y-Aib-E-G-T-F-T-S-D-Y-S-I-Aib-L-E-K-Q-A-Q-Aib-E-F-V-R-H-L-I-N-K(1,3)-Aib-T- | 56 |
| | R-Q-R-Y-NH2.C18DA-gGlu-gGlu-gGlu-gGlu-gGlu-gGlu(1,2) | |
| 57 | Y-Aib-E-G-T-F-T-S-D-Y-S-I-L-L-E-K-Q-A-Q-Aib-A-F-V-R-H-L-I-N-K(1,3)-Aib-T-R- | 57 |
| | Q-R-Y-NH2.C20DA-gGlu-gGlu-gGlu-gGlu-gGlu-gGlu(1,2) | |
| 58 | Y-Aib-E-G-T-F-T-S-D-Y-S-I-Aib-L-E-K-Q-A-Q-Aib-A-F-V-R-H-L-Y-N-K(1,3)-Aib-T- | 58 |
| | NR-Q-R-Y-H2.C18DA-gGlu-gGlu-gGlu-gGlu-gGlu-gGlu-eLys(1,2) | |
| 59 | Y-Aib-E-G-T-F-T-S-D-Y-S-I-Aib-L-E-K-Q-A-Q-Aib-A-F-V-R-H-L-I-N-K(1,3)-Aib-T- | 59 |
| | R-Q-R-Y-NH2.C20DA-gGlu-gGlu-gGlu-gGlu-gGlu-eLys(1,2) | |
| 60 | Y-Aib-E-G-T-F-T-S-D-L-S-I-Aib-L-E-K-Q-A-Q-Aib-A-F-V-R-H-L-I-N-K(1,3)-Aib-T- | 60 |
| | R-Q-R-Y-NH2.C20DA-gGlu-gGlu-gGlu-gGlu-gGlu-eLys-gGlu(1,2) | |
| 61 | Y-Aib-E-G-T-F-T-S-D-Y-S-I-Aib-L-E-K-Q-A-Q-A-A-F-V-R-H-L-I-N-K(1,3)-Aib-T-R- | 61 |
| | Q-R-Y-NH2.C18DA-gGlu-gGlu-gGlu-gGlu-gGlu-gGlu-eLys(1,2) | |
| 62 | Y-Aib-E-G-T-F-T-S-D-Y-S-I-Aib-L-E-K-Q-A-Q-Aib-A-F-V-R-H-L-I-N-K(1,3)-Aib-T- | 62 |
| | R-Q-R-Y-NH2.C18DA-gGlu-gGlu-gGlu-gGlu-gGlu(1,2) | |
| 63 | Y-Aib-E-G-T-F-T-S-D-Y-S-I-Aib-L-E-LysAc-Q-A-Q-Aib-A-F-V-R-H-L-I-N-K(1,3)- | 63 |
| | Aib-T-R-Q-R-Y-NH2.C18DA-gGlu-gGlu-gGlu-gGlu(1,2) | |
| 64 | Y-Aib-E-G-T-F-T-S-D-Y-S-I-Aib-L-E-E-E-A-Q-Aib-E-F-V-R-H-L-I-N-K(1,3)-Aib-T- | 64 |
| | R-Q-R-Y-NH2.C18DA-gGlu-gGlu-gGlu-gGlu(1,2) | |
| 65 | Y-Aib-E-G-T-F-T-S-D-Y-S-I-Aib-L-E-L-Q-A-Q-Aib-A-F-V-R-H-L-I-N-K(1,3)-Aib-T- | 65 |
| | R-Q-R-Y-NH2.C18DA-gGlu-gGlu-gGlu-gGlu-gGlu-gGlu-eLys(1,2) | |
| 66 | Y-Aib-E-G-T-F-T-S-D-Y-S-I-Aib-L-E-K-Q-A-Q-Aib-A-F-V-R-H-L-I-N-K(1,3)-I-T-R- | 66 |
| | Q-R-Y-NH2.C18DA-gGlu-gGlu-gGlu-gGlu-gGlu-gGlu-eLys(1,2) | |
| 67 | Y-Aib-E-G-T-F-T-S-D-Y-S-I-aMeL-L-D-K-Q-A-Q-Aib-A-F-V-R-H-L-I-N-K(1,3)-Aib- | 67 |
| | T-R-Q-R-Y-NH2.C18DA-gGlu-gGlu-gGlu-gGlu-gGlu-gGlu-eLys(1,2) | |
| 68 | Y-Aib-E-G-T-F-T-S-D-Y-S-I-Aib-L-E-K-Q-A-Q-Aib-A-F-V-R-H-L-I-A-K(1,3)-Aib-T- | 68 |
| | R-Q-R-Y-NH2.C18DA-gGlu-gGlu-gGlu-gGlu-gGlu-gGlu-eLys(1,2) | |
| 69 | Y-Aib-E-G-T-F-T-S-D-Y-S-I-Aib-L-E-K-Q-A-Q-Aib-A-F-V-R-H-L-I-N-K(1,3)-L-T-R- | 69 |
| | Q-R-Y-NH2.C18DA-gGlu-gGlu-Glu-gGlu-gGlu-AHX(1,2) | |
| 70 | Y-Aib-E-G-T-F-T-S-D-Y-S-I-Aib-L-D-Q-Q-A-Q-Aib-A-F-V-R-H-L-I-N-K(1,3)-Aib-T- | 70 |
| | R-Q-R-Y-NH2.C18DA-gGlu-gGlu-gGlu-gGlu-gGlu-eLys(1,2) | |
| 71 | Y-Aib-E-G-T-F-T-S-D-Y-S-I-Aib-L-E-E-Q-A-Q-Aib-A-F-V-R-H-L-I-N-K(1,3)-Aib-T- | 71 |
| | R-Q-R-Y-NH2.C18DA-gGlu-gGlu-gGlu-gGlu-gGlu-gGlu-eLys(1,2) | |
| 72 | Y-Aib-E-G-T-F-T-S-D-Y-S-I-Aib-L-E-K-Q-A-Q-Aib-A-F-V-R-H-L-I-N-K(1,3)-Aib-T- | 72 |
| | NR-Q-ArgMe-Y-H2.C18DA-gGlu-gGlu-gGlu-gGlu-Glu-gGlu-eLys(1,2) | |
| 73 | Y-Aib-E-G-T-F-T-S-D-Y-S-I-Aib-L-E-K-Q-A-Q-Aib-E-F-V-R-H-L-I-N-K(1,3)-Aib-T- | 73 |
| | R-Q-R-Y-NH2.C18DA-gGlu-gGlu-gGlu-gGlu-OEG-OEG(1,2) | |
| 74 | Y-Aib-E-G-T-F-T-S-D-Y-S-I-Aib-L-D-K-Q-A-Q-Aib-A-F-V-R-H-L-I-N-K(1,3)-Aib-T- | 74 |
| | R-Q-R-Y-NH2.C20DA-gGlu-gGlu-gGlu-gGlu-gGlu-gGlu-eLys(1,2) | |
| 75 | Y-Aib-E-G-T-F-T-S-D-Y-S-I-Aib-L-E-E-Q-A-Q-Aib-A-F-V-R-H-L-I-N-K(1,3)-Aib-T- | 75 |
| | R-Q-R-Y-NH2.C18DA-gGlu-gGlu-gGlu-gGlu-gGlu(1,2) | |
| 76 | Y-Aib-E-G-T-F-T-S-D-Y-S-I-aMeL-L-E-K-Q-A-Q-Aib-A-F-I-R-H-L-I-N-K(1,3)-Aib- | 76 |
| | T-R-Q-R-Y-NH2.C18DA-gGlu-gGlu-gGlu-gGlu-gGlu-gGlu-eLys(1,2) | |
| 77 | Y-Aib-E-G-T-F-T-S-D-Y-S-I-Aib-L-E-K-Q-A-Q-Aib-A-F-V-R-H-L-I-N-K(1,3)-Aib-T- | 77 |
| | R-Q-R-Y-NH2.C18DA-gGlu-gGlu-gGlu-gGlu(1,2) | |
| 78 | Y-Aib-E-G-T-F-T-S-D-Y-S-I-aMeL-L-E-K-Q-A-Q-Aib-A-F-V-R-H-L-I-N-K(1,3)-Aib- | 78 |
| | T-R-Q-R-Y-NH2.C20DA-gGlu-gGlu-gGlu-gGlu-gGlu-gGlu-gGlu-eLys(1,2) | |
| 79 | Y-Aib-E-G-T-F-T-S-D-Y-S-I-Aib-L-E-K-Q-A-Q-Aib-E-F-V-R-H-L-I-N-K(1,3)-Aib-T- | 79 |
| | R-Q-R-Y-NH2.C18DA-gGlu-Glu-gGlu-gGlu(1,2) | |
| 80 | Y-Aib-E-G-T-F-T-S-D-Y-S-I-Aib-L-E-K-Q-A-Q-Aib-A-F-V-R-H-L-L-N-K(1,3)-Aib-T- | 80 |
| | R-Q-R-Y-NH2.C20DA-gGlu-gGlu-Glu-gGlu-gGlu-gGlu-eLys(1,2) | |
| 81 | Y-Aib-E-G-T-F-T-S-D-Y-S-I-Aib-L-D-K-Q-A-Q-Aib-A-F-I-R-H-L-I-N-K(1,3)-Aib-T- | 81 |
| | NR-Q-R-Y-H2.C20DA-gGlu-gGlu-gGlu-gGlu-gGlu-gGlu-eLys(1,2) | |
| 82 | Y-Aib-E-G-T-F-T-S-D-Y-S-I-Aib-L-E-K-Q-A-E-Aib-A-F-V-R-H-L-I-N-K(1,3)-Aib-T- | 82 |
| | R-Q-R-Y-NH2.C20DA-gGlu-gGlu-gGlu-gGlu-gGlu-gGlu-eLys-eLys(1,2) | |
| 83 | Y-Aib-E-G-T-F-T-S-D-Y-S-I-Aib-L-E-K-Q-A-Q-Aib-E-F-V-R-H-L-I-N-K(1,3)-L-T-R- | 83 |
| | Q-R-Y-NH2.C18DA-gGlu-gGlu-gGlu-gGlu-gGlu-gGlu-eLys(1,2) | |
| 84 | Y-Aib-E-G-T-F-T-S-D-Y-S-I-Aib-L-E-K-Q-A-Y-Aib-A-F-V-R-H-L-I-N-K(1,3)-Aib-T- | 84 |
| | R-Q-R-Y-NH2.C18DA-gGlu-gGlu-gGlu-gGlu-gGlu-gGlu-eLys(1,2) | |
| 85 | Y-Aib-E-G-A-F-T-S-D-Y-S-I-Aib-L-E-K-Q-A-Q-Aib-A-F-V-R-H-L-I-N-K(1,3)-Aib-T- | 85 |
| | R-Q-R-Y-NH2.C18DA-gGlu-gGlu-gGlu-gGlu-gGlu-gGlu-eLys(1,2) | |
| 86 | Y-Aib-E-G-T-F-T-S-D-Y-S-I-Aib-L-E-K-Q-A-Q-Aib-A-F-V-R-H-L-I-N-K(1,3)-Aib-T- | 86 |
| | R-Q-R-Phe4F-NH2.C18DA-gGlu-gGlu-gGlu-gGlu-gGlu-gGlu-eLys(1,2) | |
| 87 | Y-Aib-E-G-T-F-T-S-D-Y-S-I-Aib-L-E-K-Q-A-Q-Aib-A-F-V-R-H-L-I-N-K(1,3)-Aib-T- | 87 |
| | R-Q-R-Y-NH2.C18DA-gGlu-gGlu-gGlu-gGlu-gGlu-gGlu-dtOrn(1,2) | |
| 88 | Y-Aib-E-G-T-F-T-S-D-Y-S-I-Aib-L-E-K-Q-A-Q-Aib-A-F-I-R-H-L-I-N-K(1,3)-Aib-T- | 88 |
| | R-Q-R-Y-NH2.C18DA-gGlu-gGlu-gGlu-gGlu-gGlu-gGlu-eLys(1,2) | |
| 89 | Y-Aib-E-G-T-F-T-S-D-Y-S-I-aMeL-L-E-E-Q-A-Q-Aib-A-F-V-R-H-L-I-N-K(1,3)-Aib- | 89 |
| | NT-R-Q-R-Y-H2.C20DA-gGlu-gGlu-gGlu-gGlu-gGlu-gGlu-eLys(1,2) | |
| 90 | Y-Aib-E-G-T-F-T-S-D-Y-S-I-Aib-L-E-K-Q-A-Q-Aib-A-F-V-R-H-L-I-N-K(1,3)-Aib-T- | 90 |
| | R-Q-dOR-Y-NH2.C18DA-gGlu-gGlu-Glu-gGlu-gGlu-gGlu-eLys(1,2) | |
| 91 | Y-Aib-E-G-T-F-T-S-D-Y-S-I-Aib-L-E-K-Q-R-Q-Aib-A-F-V-R-H-L-I-N-K(1,3)-Aib-T- | 91 |
| | R-Q-R-Y-NH2.C18DA-gGlu-gGlu-gGlu-gGlu-gGlu-Glu-eLys(1,2) | |
| 92 | Y-Aib-E-G-T-F-T-S-D-Y-S-I-Aib-L-E-K-Q-A-Q-Aib-A-F-V-R-H-L-I-N-K(1,3)-Aib-T- | 92 |
| | R-Q-R-Y-NH2.C18DA-gGlu-gGlu-gGlu-gGlu-gGlu-gGlu-eLys(1,2) | |
| 93 | Y-Aib-E-A-T-F-T-S-D-Y-S-I-Aib-L-E-K-Q-A-Q-Aib-A-F-V-R-H-L-I-N-K(1,3)-Aib-T- | 93 |
| | R-Q-R-Y-NH2.C18DA-gGlu-gGlu-gGlu-gGlu-gGlu-gGlu-eLys(1,2) | |
| 94 | Y-Aib-E-G-T-F-T-S-D-Y-S-I-Aib-L-E-K-Q-A-Q-Aib-A-F-V-R-H-L-I-N-K(1,3)-Aib-T- | 94 |
| | R-Q-R-Y-NH2.C20DA-gGlu-gGlu-gGlu-gGlu-gGlu-eLys-gGlu(1,2) | |
| 95 | Y-Aib-E-G-T-F-T-S-D-Y-S-I-Aib-L-E-G-Q-A-Q-Aib-A-F-V-R-H-L-I-N-K(1,3)-Aib-T- | 95 |
| | R-Q-R-Y-NH2.C18DA-gGlu-gGlu-gGlu-gGlu-gGlu-gGlu-eLys(1,2) | |
| 96 | Y-Aib-E-G-T-F-T-S-D-Y-S-I-Aib-L-E-K-Q-A-Q-Aib-A-F-V-R-H-L-I-N-K(1,3)-Aib-T- | 96 |
| | R-Q-dOR-Y-NH2.C18DA-gGlu-gGlu-gGlu-gGlu-gGlu(1,2) | |
| 97 | Y-Aib-E-G-T-F-T-S-D-Y-S-I-Aib-L-E-K-Q-A-Q-Aib-A-F-V-R-H-L-I-N-K(1,3)-Aib-T- | 97 |
| | R-Q-R-Y-NH2.C20DA-gGlu-gGlu-Glu-Glu-gGlu-gGlu-dtOrn(1,2) | |
| 98 | Y-Aib-E-G-T-F-T-S-D-Y-S-I-Aib-L-E-K-Q-A-Q-Aib-E-F-V-R-H-L-I-N-K(1,3)-Aib-T- | 98 |
| | R-Q-R-Y-NH2.C18DA-gGlu-gGlu-gGlu-gGlu-gGlu-OEG-OEG(1,2) | |
| 99 | Y-Aib-E-G-T-F-T-S-D-Y-S-I-Aib-L-E-K-Q-A-Q-Aib-A-F-V-R-H-L-I-N-K(1,3)-Aib-T- | 99 |
| | R-Q-R-Y-NH2.C20DA-gGlu-gGlu-gGlu-gGlu-gGlu-gGlu-OEG(1,2) | |
| 100 | Y-Aib-E-G-T-F-T-S-D-Y-S-I-Aib-L-E-K-E-A-Q-Aib-E-F-V-R-H-L-I-N-K(1,3)-Aib-T- | 100 |
| | R-Q-R-Y-NH2.C18DA-gGlu-gGlu-gGlu-gGlu(1,2) | |
| 101 | Y-Aib-E-G-T-F-T-S-D-Y-S-I-Aib-L-E-K-Q-A-Q-Aib-A-F-V-R-H-L-I-N-K(1,3)-Aib-T- | 101 |
| | R-Q-R-Y-NH2.C20DA-gGlu-gGlu-gGlu-gGlu-Glu-gGlu-gGlu(1,2) | |
| 102 | Y-Aib-E-G-T-F-T-S-D-Y-S-I-Aib-L-D-K-Q-A-Q-Aib-A-F-I-R-H-L-I-N-K(1,3)-Aib-T- | 102 |
| | R-Q-R-Y-NH2.C18DA-gGlu-gGlu-gGlu-gGlu-gGlu-Glu-eLys(1,2) | |
| 103 | Y-Aib-E-G-T-F-T-S-D-Y-S-I-aMeL-L-E-K-Q-A-E-Aib-A-F-V-R-H-L-I-N-K(1,3)-Aib-T- | 103 |
| | R-Q-R-Y-NH2.C20DA-gGlu-gGlu-gGlu-gGlu-gGlu-gGlu-eLys(1,2) | |
| 104 | Y-Aib-E-G-T-F-T-S-D-Y-S-I-Aib-L-E-K-Q-A-E-Aib-A-F-V-R-H-L-I-N-K(1,3)-Aib-T- | 104 |
| | R-Q-R-Y-NH2.C20DA-gGlu-gGlu-gGlu-gGlu-gGlu-gGlu-eLys(1,2) | |
| 105 | Y-Aib-E-G-T-F-T-S-D-Y-S-I-Aib-L-D-K-Q-A-Q-Aib-A-F-V-R-H-L-I-N-K(1,3)-Aib-T- | 105 |
| | R-Q-R-Y-NH2.C18DA-gGlu-gGlu-gGlu-gGlu-gGlu-gGlu-eLys(1,2) | |
| 106 | Y-Aib-E-G-T-F-T-S-D-Y-S-I-Aib-L-E-K-Q-A-Q-Aib-A-F-V-R-H-L-I-N-K(1,3)-Aib-T- | 106 |
| | R-Q-R-Y-NH2.C20DA-gGlu-gGlu-gGlu-gGlu-gGlu-AHX-AHX(1,2) | |
| 107 | Y-Aib-E-G-T-F-T-S-D-Y-S-I-Aib-L-E-K-Q-A-Q-Aib-A-F-V-R-H-L-L-N-K(1,3)-Aib-T- | 107 |
| | NR-Q-R-Y-H2.C18DA-gGlu-gGlu-gGlu-gGlu-gGlu-gGlu-eLys(1,2) | |
| 108 | Y-Aib-E-G-T-F-T-S-D-Y-S-I-Aib-L-D-E-Q-A-Q-Aib-A-F-V-R-H-L-I-N-K(1,3)-Aib-T- | 108 |
| | R-Q-R-Y-NH2.C18DA-gGlu-gGlu-gGlu-Glu-gGlu-eLys(1,2) | |
| 109 | Y-Aib-E-G-T-F-T-S-D-Y-S-I-Aib-L-E-K-Q-A-Q-Aib-A-F-I-R-H-L-I-N-K(1,3)-Aib-T- | 109 |
| | R-Q-R-Y-NH2.C20DA-gGlu-gGlu-gGlu-gGlu-gGlu-gGlu-eLys(1,2) | |
| 110 | Y-Aib-E-G-T-F-T-S-D-Y-S-I-Aib-L-E-K-Q-A-Q-Aib-A-F-V-R-H-L-I-N-K(1,3)-Aib-T- | 110 |
| | R-Q-R-Y-NH2.C18DA-gGlu-gGlu-gGlu-gGlu-OEG(1,2) | |
| 111 | Y-Aib-E-G-T-F-T-S-D-Y-S-I-Aib-L-E-K-Q-A-A-Aib-A-F-V-R-H-L-I-N-K(1,3)-Aib-T- | 111 |
| | NR-Q-R-Y-H2.C18DA-gGlu-gGlu-Glu-gGlu-gGlu-gGlu-eLys(1,2) | |
| 112 | Y-Aib-E-G-T-F-T-S-D-Y-S-E-Aib-L-E-K-Q-A-Q-Aib-A-F-V-R-H-L-I-N-K(1,3)-Aib-T- | 112 |
| | R-Q-R-Y-NH2.C18DA-gGlu-gGlu-gGlu-Glu-gGlu-gGlu-eLys(1,2) | |
| 113 | Y-Aib-E-G-T-F-T-S-D-Y-S-I-Aib-L-D-K-Q-A-Q-Aib-E-F-V-R-H-L-I-N-K(1,3)-Aib-T- | 113 |
| | R-Q-R-Y-NH2.C18DA-gGlu-gGlu-gGlu-gGlu-gGlu-eLys(1,2) | |
| 114 | Y-Aib-E-G-T-F-T-S-D-Y-S-I-Aib-L-E-K-Q-A-Q-Aib-A-F-V-R-H-L-I-N-K(1,3)-Aib-T- | 114 |
| | R-NMeQ-R-Y-NH2.C20DA-gGlu-gGlu-gGlu-gGlu-gGlu-gGlu(1,2) | |
| 115 | Y-Aib-E-G-T-F-T-S-D-L-S-I-Aib-L-E-K-Q-A-Q-Aib-A-F-V-R-H-L-L-N-K(1,3)-Aib-T- | 115 |
| | R-Q-R-Y-NH2.C20DA-gGlu-gGlu-gGlu-gGlu-gGlu-gGlu-eLys(1,2) | |
| 116 | Y-Aib-E-G-T-F-T-S-D-Y-S-I-Aib-L-E-K-Q-A-Q-Aib-A-F-V-R-H-L-I-N-K(1,3)-Aib-T- | 116 |
| | NR-Q-R-NMeY-H2.C18DA-gGlu-gGlu-gGlu-gGlu-gGlu-gGlu-eLys(1,2) | |
| 117 | Y-Aib-E-G-T-F-T-S-D-Y-S-I-Aib-L-E-K-Q-A-Q-Aib-A-F-V-R-H-L-I-N-K(1,3)-Aib-T- | 117 |
| | R-Q-R-Y-NH2.C18DA-gGlu-gGlu-gGlu-gGlu-gGlu-OEG-OEG(1,2) | |
| 118 | Y-Aib-E-G-T-F-T-S-D-Y-S-I-Aib-L-E-K-Q-A-Q-Aib-A-F-V-R-H-L-I-N-K(1,3)-Aib-T- | 118 |
| | R-Q-R-Y-NH2.C20DA-TRX-gGlu-gGlu-gGlu-gGlu-gGlu-gGlu-eLys(1,2) | |
| 119 | Y-Aib-E-G-T-F-T-S-D-Y-S-I-aMeL-L-E-K-Q-A-Q-Aib-A-F-V-R-H-L-I-N-K(1,3)-Aib- | 119 |
| | T-R-Q-R-Y-NH2.C18DA-gGlu-gGlu-gGlu-gGlu-gGlu-gGlu-eLys(1,2) | |
| 120 | Y-Aib-E-G-T-F-T-S-D-Y-S-I-Aib-L-E-K-Q-A-Q-Aib-A-F-V-R-H-L-I-N-K(1,3)-Aib-T- | 120 |
| | R-Q-R-Y-NH2.C18DA-gGlu-gGlu-Glu-gGlu-gGlu-gGlu-gDab(1,2) | |
| 121 | Y-Aib-E-G-T-F-T-S-D-Y-S-I-Aib-L-E-K-Q-A-Q-Aib-A-F-V-R-H-L-I-N-K(1,3)-Aib-T- | 121 |
| | R-Q-R-Y-NH2.C18DA-gGlu-gGlu-gGlu-gGlu-gGlu-gGlu-gGlu-eLys(1,2) | |
| 122 | Y-Aib-E-G-T-F-T-S-D-Y-S-I-L-L-E-K-Q-A-Q-Aib-A-F-V-R-H-L-I-N-K(1,3)-Aib-T-R- | 122 |
| | Q-R-Y-NH2.C18DA-gGlu-gGlu-gGlu-gGlu-gGlu(1,2) | |
| 123 | Y-Aib-E-G-T-F-T-S-D-Y-S-I-Aib-L-E-A-Q-A-Q-Aib-A-F-V-R-H-L-I-N-K(1,3)-Aib-T- | 123 |
| | R-Q-R-Y-NH2.C18DA-gGlu-gGlu-gGlu-gGlu-gGlu-gGlu-eLys(1,2) | |
| 124 | Y-Aib-E-G-T-F-T-S-D-Y-S-I-Aib-L-E-R-Q-A-Q-Aib-A-F-V-R-H-L-I-N-K(1,3)-Aib-T- | 124 |
| | R-Q-R-Y-NH2.C18DA-gGlu-gGlu-gGlu-gGlu-gGlu-gGlu-eLys(1,2) | |
| 125 | Y-Aib-E-G-T-F-T-S-D-Y-S-I-Aib-L-E-K-Q-A-Q-Aib-A-F-V-R-H-L-I-N-K(1,3)-Aib-T- | 125 |
| | R-Q-R-Y-NH2.C20DA-gGlu-gGlu-gGlu-gGlu-gGlu-gGlu-eLys(1,2) | |
| 126 | Y-Aib-E-G-T-F-T-S-D-Y-S-I-Aib-L-E-K-Q-A-Q-Aib-A-F-V-R-H-L-I-N-K(1,3)-V-T-R- | 126 |
| | Q-R-Y-NH2.C18DA-gGlu-gGlu-gGlu-gGlu-gGlu-Glu-eLys(1,2) | |
| 127 | Y-Aib-E-G-T-F-T-S-D-Y-S-I-Aib-L-E-K-Q-A-Q-Aib-A-F-V-R-H-L-I-N-K(1,3)-L-T-R- | 127 |
| | Q-R-Y-NH2.C18DA-gGlu-gGlu-gGlu-gGlu-gGlu-gGlu-eLys(1,2) | |
| 128 | Y-Aib-E-G-T-F-T-S-D-Y-S-I-Aib-L-E-K-Q-A-Q-Aib-A-F-V-R-A-L-I-N-K(1,3)-Aib-T- | 128 |
| | R-Q-R-Y-NH2.C18DA-gGlu-gGlu-gGlu-gGlu-gGlu-gGlu-eLys(1,2) | |
| 129 | Y-Aib-E-G-T-F-T-S-D-Y-S-I-Aib-L-E-E-Q-A-Q-Aib-E-F-V-R-H-L-I-N-K(1,3)-Aib-T- | 129 |
| | R-Q-R-Y-NH2.C18DA-gGlu-gGlu-gGlu-gGlu(1,2) | |
| 130 | Y-Aib-E-G-T-F-T-S-D-Y-S-I-Aib-L-E-E-E-A-Q-Aib-E-F-V-R-H-L-I-N-K(1,3)-Aib-T- | 130 |
| | R-Q-R-Y-NH2.C18DA-gGlu-OEG-OEG(1,2) | |
| 131 | Y-Aib-E-G-T-F-T-S-D-Y-S-I-Aib-L-D-K-Q-A-Q-Aib-E-F-V-R-H-L-I-N-K(1,3)-Aib-T- | 131 |
| | R-Q-R-Y-NH2.C18DA-gGlu-gGlu-gGlu-gGlu-gGlu-gGlu-eLys(1,2) | |
| 132 | Y-Aib-E-G-T-F-T-S-D-Y-S-I-Aib-L-E-K-Q-A-Q-Aib-A-F-V-R-H-L-I-N-K(1,3)-Aib-T- | 132 |
| | R-Q-R-Y-NH2.C20DA-gGlu-Glu-gGlu-eLys-gGlu-gGlu-gGlu(1,2) | |
| 133 | Y-Aib-E-G-T-F-T-S-D-Y-S-I-Aib-L-E-K-Q-A-Q-Aib-A-F-V-R-H-L-I-N-K(1,3)-Aib-T- | 133 |
| | R-Q-R-Y-NH2.C20DA-TRX-gGlu-gGlu-gGlu-gGlu-gGlu-gGlu(1,2) | |
| 134 | Y-Aib-E-G-T-F-T-S-D-Y-S-I-Aib-L-E-K-Q-A-Q-Aib-A-F-V-R-H-L-I-N-K(1,3)-Aib-T- | 134 |
| | R-Q-R-Y-NH2.C18DA-gGlu-gGlu-gGlu-gGlu-gGlu-gGlu(1,2) | |
| 135 | Y-Aib-E-G-T-F-T-S-D-L-S-I-Aib-L-E-K-Q-A-Q-Aib-A-F-V-R-H-L-I-N-K(1,3)-Aib-T- | 135 |
| | R-Q-R-Y-NH2.C20DA-gGlu-gGlu-gGlu-gGlu-gGlu-gGlu-gGlu-eLys(1,2) | |
| 136 | Y-Aib-E-G-T-F-T-S-D-Y-S-I-Aib-L-E-K-Q-A-Q-Aib-A-F-V-R-H-L-I-N-K(1,3)-Aib-T- | 136 |
| | R-Q-R-Y-NH2.C20DA-gGlu-gGlu-gGlu-gGlu-OEG-gGlu-gGlu(1,2) | |
| 137 | Y-Aib-E-G-T-F-T-S-D-Y-S-I-Aib-L-E-K-Q-A-Q-Aib-A-F-V-R-H-L-I-N-K(1,3)-Aib-T- | 137 |
| | R-Q-R-Y-NH2.C18DA-TRX-gGlu-gGlu-gGlu-gGlu-gGlu-gGlu-eLys(1,2) | |
| 138 | Y-Aib-E-G-T-F-T-S-D-Y-S-I-Aib-L-D-E-Q-A-Q-Aib-A-F-V-R-H-L-I-N-K(1,3)-Aib-T- | 138 |
| | R-Q-R-Y-NH2.C20DA-gGlu-gGlu-gGlu-gGlu-gGlu-eLys(1,2) | |
| 139 | Y-Aib-E-G-T-F-T-S-D-Y-S-I-Aib-L-E-K-Q-A-Q-Aib-A-F-V-R-H-A-I-N-K(1,3)-Aib-T- | 139 |
| | R-Q-R-Y-NH2.C18DA-gGlu-gGlu-gGlu-gGlu-gGlu-gGlu-eLys(1,2) | |
| 140 | Y-Aib-E-G-T-F-T-S-D-Y-S-I-Aib-L-E-K-Q-A-Q-Aib-A-F-V-R-H-L-I-N-K(1,3)-Aib-T- | 140 |
| | R-Q-R-Y-NH2.C18DA-gGlu-Glu-gGlu-gGlu-gGlu-OEG(1,2) | |
| 141 | Y-Aib-E-G-T-F-T-S-D-Y-S-I-Aib-L-E-K-Q-A-Q-Aib-A-F-V-R-H-L-I-N-K(1,3)-Aib-T- | 141 |
| | R-Q-R-Y-NH2.C18DA-gGlu-gGlu-Glu-Glu-gGlu-eLys(1,2) | |
| 142 | Y-Aib-E-G-T-F-T-S-D-Y-A-I-Aib-L-E-K-Q-A-Q-Aib-A-F-V-R-H-L-I-N-K(1,3)-Aib-T- | 142 |
| | R-Q-R-Y-NH2.C18DA-gGlu-gGlu-gGlu-gGlu-gGlu-gGlu-eLys(1,2) | |
| 143 | Y-Aib-E-G-T-F-T-S-D-Y-S-I-Aib-L-E-K-A-A-Q-Aib-A-F-V-R-H-L-I-N-K(1,3)-Aib-T- | 143 |
| | R-Q-R-Y-NH2.C18DA-gGlu-gGlu-gGlu-gGlu-gGlu-gGlu-eLys(1,2) | |
| 144 | Y-Aib-E-G-T-F-T-S-D-Y-S-I-Aib-L-E-K-Q-A-Q-Aib-S-F-V-R-H-L-I-N-K(1,3)-Aib-T- | 144 |
| | R-Q-R-Y-NH2.C18DA-gGlu-gGlu-gGlu-gGlu-gGlu-gGlu-eLys(1,2) | |
| 145 | Y-Aib-E-G-T-F-T-S-D-Y-S-I-Aib-L-E-K-Q-A-Q-Aib-E-F-V-R-H-L-I-N-K(1,3)-Aib-T- | 145 |
| | R-Q-R-Y-NH2.C18DA-gGlu-gGlu-gGlu-gGlu-gGlu-gGlu-eLys(1,2) | |
| 146 | Y-Aib-E-G-T-F-T-S-D-Y-S-I-Aib-L-E-K-Q-A-Q-Aib-A-F-V-R-H-L-I-N-K(1,3)-Aib-T- | 146 |
| | R-Q-bhArg-Y-NH2.C18DA-gGlu-gGlu-gGlu-gGlu-gGlu-gGlu-eLys(1,2) | |
| 147 | Y-Aib-E-G-T-F-T-S-D-Y-S-I-Aib-L-D-R-Q-A-Q-Aib-A-F-V-R-H-L-I-N-K(1,3)-Aib-T- | 147 |
| | R-Q-R-Y-NH2.C18DA-gGlu-gGlu-gGlu-gGlu-gGlu-gGlu-eLys(1,2) | |
| 148 | Y-Aib-E-G-T-F-T-S-D-Y-S-I-Aib-L-E-K-Q-A-E-Aib-A-F-V-R-H-L-I-N-K(1,3)-Aib-T- | 148 |
| | R-Q-R-Y-NH2.C20DA-gGlu-gGlu-gGlu-gGlu-OEG-gGlu-eLys(1,2) | |
| 149 | Y-Aib-E-G-T-F-T-S-D-Y-S-I-Aib-L-E-K-Q-A-Q-Aib-E-F-V-R-H-L-I-N-K(1,3)-Aib-T- | 149 |
| | R-Q-R-Y-NH2.C20DA-gGlu-gGlu-gGlu-gGlu-gGlu-gGlu-eLys(1,2) | |
| 150 | Y-Aib-E-G-T-F-T-S-D-Y-S-I-aMeL-L-E-K-Q-A-Q-Aib-A-F-V-R-H-L-I-N-K(1,3)-Aib- | 150 |
| | T-R-Q-R-Y-NH2.C20DA-gGlu-gGlu-gGlu-gGlu-gGlu-gGlu-eLys(1,2) | |
| 151 | Y-Aib-E-G-T-F-T-S-D-Y-S-I-Aib-L-E-K-Q-A-Q-Y-A-F-V-R-H-L-I-N-K(1,3)-Aib-T-R- | 151 |
| | Q-R-Y-NH2.C18DA-gGlu-gGlu-gGlu-gGlu-gGlu-gGlu-eLys(1,2) | |
| 152 | Y-Aib-E-G-T-F-T-S-D-Y-S-I-Aib-L-E-K-Q-A-Q-Aib-E-F-V-R-H-L-I-N-K(1,3)-Aib-T- | 152 |
| | R-Q-R-Y-NH2.C18DA-gGlu-Glu-gGlu-gGlu-gGlu-gGlu-eLys-eLys(1,2) | |
| 153 | Y-Aib-E-G-T-F-T-S-D-Y-S-I-Aib-L-D-K-Q-A-Q-Aib-A-F-V-R-Y-L-I-N-K(1,3)-Aib-T- | 153 |
| | R-Q-R-Y-NH2.C18DA-gGlu-gGlu-gGlu-gGlu-gGlu-gGlu-eLys(1,2) | |
| 154 | Y-Aib-E-G-T-F-T-S-D-Y-S-I-Aib-L-E-K-Q-A-Q-Aib-A-F-V-R-H-L-I-N-K(1,3)-Aib-T- | 154 |
| | R-Q-R-Y-NH2.C18DA-gGlu-gGlu-gGlu-Glu-gGlu-gGlu-gGlu-AHX(1,2) | |
| 155 | Y-Aib-E-G-T-F-T-S-D-Y-S-I-Aib-L-D-E-Q-A-Q-Aib-A-F-V-R-H-L-I-N-K(1,3)-Aib-T- | 155 |
| | R-Q-R-Y-NH2.C20DA-gGlu-gGlu-gGlu-gGlu-gGlu-eLys-eLys(1,2) | |
| 156 | Y-Aib-E-G-T-F-T-S-D-Y-S-A-Aib-L-E-K-Q-A-Q-Aib-A-F-V-R-H-L-I-N-K(1,3)-Aib-T- | 156 |
| | R-Q-R-Y-NH2.C18DA-gGlu-gGlu-gGlu-gGlu-gGlu-Glu-eLys(1,2) | |
| 157 | Y-Aib-E-G-T-F-T-S-D-Y-S-I-Aib-L-E-K-Q-A-Q-Aib-A-F-V-R-H-L-I-N-K(1,3)-Aib-T- | 157 |
| | R-Q-R-Y-NH2.C20DA-gGlu-gGlu-gGlu-gGlu-gGlu-gGlu-AHX(1,2) | |
| 158 | Y-Aib-E-G-T-F-T-S-D-Y-S-I-Aib-L-E-K-Q-A-Q-Aib-A-F-V-R-H-L-I-N-K(1,3)-L-T-R- | 158 |
| | Q-R-Y-NH2.C18DA-gGlu-gGlu-gGlu-gGlu-gGlu(1,2) | |
| 159 | Y-Aib-E-G-T-F-T-S-D-Y-S-I-Aib-L-E-K-Q-A-Q-Aib-A-F-V-R-H-L-I-N-K(1,3)-A-T-R- | 159 |
| | Q-R-Y-NH2.C18DA-gGlu-gGlu-gGlu-gGlu-gGlu-gGlu-eLys(1,2) | |
| 160 | Y-Aib-E-G-T-F-T-S-D-Y-S-I-E-L-E-K-Q-A-Q-Aib-A-F-V-R-H-L-I-N-K(1,3)-Aib-T-R- | 160 |
| | Q-R-Y-NH2.C18DA-gGlu-gGlu-gGlu-gGlu-gGlu-gGlu-eLys(1,2) | |
| 161 | Y-Aib-E-G-T-F-T-S-D-Y-S-I-Aib-L-E-K-Q-A-E-Aib-A-F-V-R-H-L-I-N-K(1,3)-Aib-T- | 161 |
| | R-Q-R-Y-NH2.C20DA-gGlu-Glu-gGlu-gGlu-gGlu-OEG-eLys(1,2) | |
| 162 | Y-Aib-E-G-T-F-T-S-D-Y-S-I-Aib-L-E-K-Q-A-E-Aib-A-F-V-R-H-L-I-N-K(1,3)-Aib-T- | 162 |
| | R-Q-R-Y-NH2.C20DA-gGlu-gGlu-gGlu-OEG-gGlu-gGlu-eLys(1,2) | |
| 163 | Y-Aib-E-G-T-F-T-S-D-Y-S-I-Aib-L-E-K-Q-A-Q-Aib-E-F-V-R-H-L-I-N-K(1,3)-Aib-T- | 163 |
| | R-Q-R-Y-NH2.C18DA-gGlu-gGlu-gGlu-gGlu-eLys(1,2) | |
| 164 | Y-Aib-E-G-T-F-T-S-D-Y-S-I-Aib-L-E-K-E-A-Q-Aib-A-F-V-R-H-L-I-N-K(1,3)-Aib-T- | 164 |
| | R-Q-R-Y-NH2.C18DA-gGlu-gGlu-gGlu-gGlu-gGlu-Glu-eLys(1,2) | |
| 165 | Y-Aib-E-G-T-F-T-S-D-Y-S-I-A-L-E-K-Q-A-Q-Aib-A-F-V-R-H-L-I-N-K(1,3)-Aib-T-R- | 165 |
| | Q-R-Y-NH2.C18DA-gGlu-gGlu-gGlu-gGlu-gGlu-gGlu-eLys(1,2) | |
| 166 | Y-Aib-E-G-T-F-T-S-D-L-S-I-Aib-L-E-K-Q-A-Q-Aib-A-F-V-R-H-L-I-N-K(1,3)-Aib-T- | 166 |
| | R-Q-R-Y-NH2.C20DA-gGlu-gGlu-gGlu-Glu-eLys-gGlu-gGlu(1,2) | |
| 167 | Y-Aib-E-G-T-F-T-S-D-Y-S-I-Aib-L-E-K-Q-A-Q-Aib-A-F-V-R-H-L-I-N-K(1,3)-Aib-T- | 167 |
| | R-Q-R-Y-NH2.C20DA-gGlu-gGlu-gGlu-gGlu-gGlu-AHX(1,2) | |
| 168 | Y-Aib-E-G-T-F-T-S-D-Y-S-I-Aib-L-E-K-Q-A-Q-Aib-A-F-V-R-H-L-I-N-K(1,3)-Aib-T- | 168 |
| | R-Q-R-Y-NH2.C18DA-gGlu-gGlu-gGlu-eLys-gGlu-gGlu-gGlu(1,2) | |
| 169 | Y-Aib-E-G-T-F-T-S-D-L-S-I-Aib-L-E-K-Q-A-Q-Aib-A-F-V-R-H-L-I-N-K(1,3)-Aib-T- | 169 |
| | R-Q-R-Y-NH2.C18DA-gGlu-gGlu-Glu-gGlu-gGlu-gGlu-eLys(1,2) | |
| 170 | Y-Aib-E-G-T-F-T-S-D-Y-S-I-Aib-L-E-K-Q-A-Q-E-A-F-V-R-H-L-I-N-K(1,3)-Aib-T-R- | 170 |
| | Q-R-Y-NH2.C18DA-gGlu-gGlu-gGlu-Glu-gGlu-gGlu-eLys(1,2) | |
| 171 | Y-Aib-E-G-T-F-T-S-D-Y-S-I-Aib-L-E-R-Q-A-Q-Aib-A-F-V-R-H-L-I-N-K(1,3)-Aib-T- | 171 |
| | R-Q-R-Y-NH2.C20DA-gGlu-gGlu-gGlu-gGlu-gGlu-gGlu-eLys(1,2) | |
| 172 | Y-Aib-E-G-T-F-T-S-D-Y-S-I-Aib-L-E-K-Q-A-Q-Aib-A-F-V-R-H-L-I-N-K(1,3)-W-T-R- | 172 |
| | Q-R-Y-NH2.C18DA-gGlu-gGlu-gGlu-gGlu-gGlu-gGlu-eLys(1,2) | |
| 173 | Y-Aib-E-G-T-F-T-S-D-Y-S-I-Aib-L-D-K-Q-A-Q-Aib-A-F-V-R-H-L-L-N-K(1,3)-Aib-T- | 173 |
| | R-Q-R-Y-NH2.C18DA-gGlu-gGlu-gGlu-gGlu-gGlu-Glu-eLys(1,2) | |
| 174 | Y-Aib-E-G-T-F-T-S-D-Y-S-I-Aib-L-E-K-Q-A-Q-Aib-A-F-V-R-H-L-I-N-K(1,3)-Aib-T- | 174 |
| | R-Q-R-Y-NH2.C18DA-gGlu-gGlu-gGlu-gGlu-Glu-eLys-gGlu(1,2) | |
| 175 | Y-Aib-E-G-T-F-T-S-D-Y-S-I-Aib-L-E-K-Q-A-Q-Aib-A-F-V-R-H-L-I-N-K(1,3)-Aib-T- | 175 |
| | R-Q-R-Y-NH2.C20DA-gGlu-gGlu-gGlu-gGlu-AHX-gGlu-AHX(1,2) | |
| 176 | Y-Aib-E-G-T-F-T-S-D-Y-S-I-Aib-L-E-K-Q-A-Q-Aib-A-F-V-R-H-L-I-N-K(1,3)-Aib-T- | 176 |
| | R-Q-R-Y-NH2.C20DA-gGlu-gGlu-gGlu-gGlu-gGlu(1,2) | |

### Reference Compound

TABLE B

| Compound | | SEQ |
|---|---|---|
| No | | ID |
| Reference | | NOS |
| Ref.1# | NMeY-Aib-E-G-T-aMeF-T-S-D-K(1,3)-S-I-Aib-L-E-K-Q-R-Q-Iva-E-F-V-R-H-L-L-N-K- | 177, |
| | Aib-T-R-Q-R-Y-NH2.C16A-G-G-G-G(1,2) | 178 |
| Ref.2## | NMeY-Aib-E-G-T-aMeF-T-S-D-K(1,3)-S-I-Aib-L-E-K-Q-R-Q-Iva-E-F-V-R-H-L-L-N-K- | 179, |
| | Aib-T-R-Q-R-Y-NH2.C18DA-G-G-G-G(1,2) | 180 |
| Ref.3### | Y-Aib-E-G-T-aMeF-T-S-D-K(1,3)-S-I-Aib-L-E-K-Q-R-Q-Iva-E-F-V-R-H-L-L-N-K- | 188, |
| | Aib-T-R-Q-R-Y-NH2.C18DA-G-G-G-G(1,2) | 189 |

#Ref. 1 is Example 41 of EP3467106 A1; ##Ref. 2 is Example 42 of EP3467106 A1; ##Ref. 3 is Example 40 of EP3467106 A1.

The compounds of the invention according to general formula (I) and any embodiment disclosed herewith bind to and/or activate the GLP-1, the GIP and the NPY2 receptors.

The compounds of the present invention according to general formula (I) are able to agonize the hGLP1R, the hGIPR and the hNPY2R in the presence of human serum albumin (see table 1), the main protein present in human plasma. Activity of peptides of the invention in the presence of human plasma is an important prerequisite for viable compounds useful for the treatment of the disclosed diseases. The skilled person will be aware of suitable assay formats, and an example is provided hereinafter. For instance, for peptides of the present invention the EC₅₀ values to the hGLP1R, the hGIPR and the hNPY2R in the presence of 100% human plasma (i.e. 100% hP) was evaluated by the assay as described in Example 1 hereinafter.

The compounds of the present invention show an EC₅₀ towards hGLP1R 100% hP and hGIPR 100% hP below 100 nM (e.g. 0.1 nM to 100 nM), and towards hNPY2R 100% hP below 1000 nM (e.g. 1 nM to 1000 nM). In a preferred embodiment of compounds of the present invention, the EC₅₀ towards hGLP1R 100% hP and hGIPR 100% hP is below or equal to 30 nM (e.g. 0.1 nM to 30 nM), and towards hNPY2R 100% hP is below 600 nM (e.g. 1 nM to 600 nM).

In a highly preferred embodiment of compounds of the present invention, the EC₅₀ towards hGLP1R 100% hP and GIPR 100% hP is below or equal to 20 nM (e.g. 0.1 nM to 20 nM), and towards hNPY2R 100% hP is below 200 nM (e.g. 1 nM to 200 nM).

Alternatively, in a highly preferred embodiment of compounds of the present invention, the EC₅₀ towards hGLP1R 100% hP and hGIPR 100% hP is below or equal to 10 nM (e.g. 0.1 nM to 10 nM), and towards hNPY2R 100% hP is below 100 nM (e.g. 1 nM to 100 nM).

The compounds of the invention according to formula (I) are soluble at or around physiological pH (see table 2), and therefore suitable for use in human therapy, preferably by administration via subcutaneous injection.

In particular, the compound of formula (I) comprising at least six (6) residues of amino acids selected from the group consisting of E and [gGlu] showed suitable solubility at or around physiological pH.

In the context of the present invention the term “at or around physiological pH” means at or around pH 7, e.g. pH 7±0.5. There are several techniques known to the skilled person in the art how to determine solubility.

Preferably the solubility of the peptide of the invention is determined as disclosed in Example 2 hereinafter.

Generally, the solubility of the compounds of the invention is greater than or equal to 1.0 mg/ml at or around physiological pH.

In a preferred embodiment, the solubility of the compounds of the invention is greater than or equal to 3.0 mg/ml at or around physiological pH.

In a highly preferred embodiment, the solubility of the compounds of the invention is greater than or equal to 5.0 mg/ml at or around physiological pH.

In some further embodiments, the solubility at or around physiological pH of the compounds of the invention is equal or greater than 6.0 mg/ml, 7.0 mg/ml, 8.0 mg/ml, or 9.0 mg/ml.

In one embodiment of the compounds of the present invention, the EC₅₀ towards hGLP1R 100% hP and hGIPR 100% hP is below or equal to 100 nM, and towards hNPY2R 100% hP is below 1000 nM, and the solubility is greater than or equal to 1.0 mg/ml at or around physiological pH.

In a preferred embodiment of the compounds of the present invention, the EC₅₀ towards hGLP1R 100% hP and hGIPR 100% hP is below or equal to 30 nM, and towards hNPY2R 100% hP is below 600 nM, and the solubility is greater than or equal to 1.0 mg/ml at or around physiological pH.

In a highly preferred embodiment of the compounds of the present invention, the EC₅₀ towards hGLP1R 100% hP and hGIPR 100% hP is below or equal to 20 nM, and towards hNPY2R 100% hP is below 200 nM, and the solubility is greater than or equal to 3.0 mg/ml at or around physiological pH. 25 In an even highly preferred embodiment of the compounds of the present invention, the EC₅₀ towards hGLP1R 100% hP and hGIPR 100% hP is below or equal to 20 nM, and towards hNPY2R 100% hP is below 200 nM, and the solubility is greater than or equal to 5.0 mg/ml at or around physiological pH.

The compounds of the invention according to general formula (I) have favourable pharmacokinetic properties, in particular suitable in-vivo half-life for use in human therapy, for example by administration via subcutaneous injection. In this regard, the in-vivo half-life of the peptides of the invention may be greater than 4 hours or greater than 6 hours or greater than 8 hours or greater than 10 hours in the mouse (measurement described in Example 3 and table 3).

In one embodiment of the compounds of the present invention, the EC₅₀ towards hGLP1R 100% hP and hGIPR 100% hP is below or equal to 100 nM (e.g. 0.01 nM to 100 nM), and towards hNPY2R 100% hP is below 1000 nM (e.g. 0.01 nM to 1000 nM), the solubility is greater than or equal to 1.0 mg/ml at or around physiological pH, and the in-vivo half-life is greater than 4 hours.

In a preferred embodiment of the peptides of the present invention, the EC₅₀ towards hGLP1R 100% hP and hGIPR 100% hP is below or equal to 60 nM (e.g. 0.01 nM to 60 nM), and towards hNPY2R 100% hP is below 600 nM (e.g. 0.01 nM to 600 nM), the solubility is greater than or equal to 1.0 mg/ml at or around physiological pH, and the in-vivo half-life is greater than 4 hours.

In a highly preferred embodiment of the peptides of the present invention, the EC₅₀ towards hGLP1R 100% hP and GIPR 100% hP is below or equal to 20 nM (e.g. 0.01 nM to 20 nM), and towards hNPY2R 100% hP is below 200 nM (e.g. 0.01 nM to 200 nM), the solubility is greater than or equal to 3.0 mg/ml at or around physiological pH, and the in-vivo half-life is greater than 6 hours.

### Pharmaceutical Compositions

In a second aspect, the invention provides pharmaceutical compositions (i.e. formulation) comprising at least one compound of general formula (I), or a pharmaceutically acceptable salt thereof, and at least one pharmaceutically acceptable carrier and/or excipient.

The term “pharmaceutically acceptable carrier and/or excipient” includes any of the standard pharmaceutical carrier and excipient well known in the pharmaceutical art and commonly used in formulations of peptides or derivative thereof, and examples may be found in “Remington's Pharmaceutical Sciences” 17^{th} edition and more recent editions thereof. The term further encompasses any carrier and/or excipients agents suitable for peptides formulation and listed in the US Pharmacopeia for use in humans.

Pharmaceutical compositions of the present invention are suitable for use in a method of preventing and/or treating diseases as disclosed hereinafter, such as obesity, diabetes, metabolic syndrome, MASH/NASH, and/or comorbidities thereof. Said pharmaceutical compositions, which are suited for administration to a human being in the need thereof, typically comprise a therapeutically effective amount of the active ingredient.

It should be appreciated that different formulation (e.g. solid or liquid formulation) may be used depending on physicochemical characteristic of the compound of the invention.

It should be appreciated that different routes of administration may be used depending on the choice of formulation and chemical and/or metabolic stability of the compound of invention. Such administration routes may include but are not limited to oral administration, parenteral administration (intravenous (IV), subcutaneous (SC), intradermal (ID) and intramuscular (IM)), or inhalation. In a preferred embodiment of the invention, the administration route is subcutaneous injection.

It should be appreciated that different pharmaceutical form (e.g. solid or liquid dosage form) may be used depending of the formulation and administration route. In a preferred aspect of the invention, the pharmaceutical form is an injectable solution (e.g. aqueous injectable solution).

The compounds of the invention may be administered subcutaneously using a suitable device such as a pre-filled ready-to-use injection device like a syringe, a pen injector, or an auto-injector.

The actual pharmaceutically effective amount or therapeutic dosage will usually depend on factors known by those skilled in the art such as age and physical condition of the patient, route of administration and severity of disease. In any case, the compounds will be administered at dosages and in a manner, which allows a pharmaceutically effective amount to be delivered based upon patient's unique condition.

### Method of Treatment/Use in Treatment

In a third aspect, the invention provides compounds according to general formula (I) or pharmaceutical acceptable salts thereof, according to any of the aspects and embodiments disclosed herein, for use as a medicament. Since NPY2 receptors have a feeding suppressive action, a polypeptide having NPY2 agonistic activity may be useful in the treatment of symptoms associated with eating disorder, obesity (e.g. simple obesity or symptomatic obesity) and/or diabetes. Furthermore, the gut hormones GLP-1 and GIP (called incretins), promote insulin secretion from the pancreas. Since incretins are closely related to glucose metabolism by their ability to stimulate insulin secrection, a polypeptide having GLP-1 receptor agonistic activity and GIP receptor agonistic activity may be useful in the treatment of symptoms associated with a glucose metabolism disorder, including diabetes and obesity. Thus, the compounds of the present invention may have a feeding suppressive action, a body weight-lowering action, and/or a reducing body fat mass action.

The invention provides compounds for use in the prevention, treatment, and/or remission of a disease, disorder, or condition selected from the list consisting of excessive body weight (body overweight), obesity (e.g. simple obesity and chronic weight management, or symptomatic obesity), eating disorders, other obesity related diseases such as osteoporosis, sleep apnoea, obesity associated cancers, and obesity associated asthma. Examples of the symptomatic obesity include endocrine obesity (e.g. Cushing syndrome, hypothyroidism, insulinoma, obese type 2 diabetes, pseudohypoparathyroidism, hypogonadism and associated endocrine disorders, such as polycystic ovary syndrome (PCOS)), central obesity (e.g., hypothalamic obesity, frontal lobe syndrome, Kleine-Levin syndrome), hereditary obesity (e.g. Prader-Willi syndrome, Laurence-Moon-Biedl syndrome), drug-induced obesity (e.g., steroid, phenothiazine, insulin, sulfonylurea agent, β-blocker-induced obesity).

The invention provides compounds for use in the prevention, treatment, and/or remission of a disease, disorder, or condition selected from the list consisting of insulin resistance, diabetes (e.g. type 1 diabetes or type 2 diabetes), prediabetes.

The invention provides compounds for use in the prevention, treatment, and/or remission of a disease, disorder, or condition selected from the list consisting of hyperlipidemia, hypertriglyceridemia, hypercholesterolemia, high LDL-cholesterolemia, low HDL-cholesterolemia, and postprandial hyperlipemia.

Further, the invention provides a compound for use in the prevention, treatment, and/or remission of metabolic syndrome (three of the following five medical conditions: abdominal obesity, high blood pressure, high blood sugar, high serum triglycerides, and low serum high-density lipoprotein (HDL)).

The invention provides compounds for use in the prevention, treatment, and/or remission of liver diseases such as metabolic dysfunction-associated fatty liver disease (MAFLD), non-alcoholic fatty liver disease (NAFLD), metabolic dysfunction-associated steatohepatitis (MASH), formerly known as non-alcoholic steatohepatitis (NASH), portal hypertension.

The invention provides compounds for use in the prevention, treatment, and/or remission of cardiovascular diseases (e.g. hypertension, atherosclerosis, stroke or cardiac failure);

The invention provides compounds for use in the prevention, treatment, and/or remission of kidney diseases such as diabetic kidney disease (DKD), and chronic kidney disease (CKD).

The invention provides compounds for use in the prevention, treatment, and/or remission of neurodegenerative diseases (Alzheimer's or Parkinson's disease).

In one preferred embodiment, compounds of the invention are suitable for use in the prevention, treatment, and/or remission of disease, disorder and/or condition selected from the list consisting of chronic weight management, excessive body weight, obesity, eating disorders, diabetes (type 1 or type 2), prediabetes, hyperlipidemia, metabolic syndrome, MAFLD/NAFLD, MASH/NASH, and cardiovascular diseases; preferably obesity, prediabetes and/or diabetes (type 1 or type 2).

The compounds of the invention may also be used for the prevention of obesity, or prevention or reversal of comorbidities of obesity and/or comorbidities of overweight, such as type 2 diabetes, high blood pressure, MAFLD/NAFLD, MASH/NASH, DKD, CKD, sleep apnoe, obesity associated cancers, or obesity associated asthma.

In a fourth aspect, the invention provides a method for the treatment of a disease, disorder and/or condition listed hereinbefore, said method comprising administering a therapeutic effective amount of a compounds of general formula (I) or pharmaceutically acceptable salts thereof, according to any of the aspects and embodiments disclosed herein, to an individual in need thereof. Preferably, the disease, disorder and/or condition is selected from the list consisting of chronic weight management, excessive body weight, obesity, eating disorders, diabetes (type 1 or type 2), prediabetes, hyperlipidemia, metabolic syndrome, MAFLD/NAFLD, MASH/NASH, and/or cardiovascular diseases. Most preferably the disease, disorder and/or condition is obesity, prediabetes and/or diabetes (type 1 or type 2).

Furthermore, the invention provides a method for the treatment of a disease, disorder and/or condition by activation of all the receptors GLP1-R, GIPR and NPY2R, said method comprising administering a therapeutic effective amount of a compound of general formula (I) or a pharmaceutically acceptable salt thereof, according to any of the aspects and embodiments disclosed herein.

Likewise, the present invention relates to the use of compounds of general formula I or a pharmaceutically acceptable salt thereof in the manufacture of a medicament for the treatment of a disease, disorder and/or condition listed hereinbefore, according to any of the aspects and embodiments disclosed herein.

The dose range of the compound according to the general formula (I) applicable per week is usually from 0.01 to 100 mg for humans (subcutaneous administration).

The compound of the invention may be administered by subcutaneous injection using a suitable device such as a pre-filled ready-to-use injection device, a syringe, a pen injector, or an auto-injector.

The actual pharmaceutically effective amount or therapeutic dosage will usually depend on factors known by those skilled in the art such as age and weight of the patient, route of administration and severity of disease. In any case, the compounds will be administered at dosages and in a manner, which allows a pharmaceutically effective amount to be delivered based upon patient's unique condition.

### EMBODIMENTS

### (Briefly, Emb.)

Emb. 1. A compound according to general formula (I),

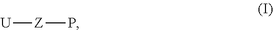

wherein P is a peptide comprising or, alternatively, consisting of the amino acid sequence,

(SEQ ID NO: 185)
Y-Aib-E-X₄-X₅-F-T-S-D-X₁₀-X₁₁-X₁₂-X₁₃-L-X₁₅-X₁₆-X₁₇-X₁₈-X₁₉-X₂₀-X₂₁-F-X₂₃-R-X₂₅-X₂₆-
X₂₇-X₂₈-K-X₃₀-T-R-X₃₃-X₃₄-X₃₅

wherein
X₄ is selected from the group consisting of A, and G,
X₅ is selected from the group consisting of A, E, and T,
X₁₀ is selected from the group consisting of F, L, and Y,
X₁₁ is selected from the group consisting of A, and S,
X₁₂ is selected from the group consisting of A, E, and I,
X₁₃ is selected from the group consisting of A, Aib, E, L, and aMeL,
X₁₅ is selected from the group consisting of D, and E,
X₁₆ is selected from the group consisting of A, Aib, E, G, K, L, LysAc, N, Q, and R,
X₁₇ is selected from the group consisting of A, E, I, and Q,
X₁₈ is selected from the group consisting of A, E and R,
X₁₉ is selected from the group consisting of A, E, Q, and Y,
X₂₀ is selected from the group consisting of A, Aib, E, and Y,
X₂₁ is selected from the group consisting of A, E, and S,
X₂₃ is selected from the group consisting of I, L, and V,
X₂₅ is selected from the group consisting of A, H, Q, and Y,
X₂₆ is selected from the group consisting of A, and L,
X₂₇ is selected from the group consisting of I, L, and Y,
X₂₈ is selected from the group consisting of A, and N,
X₃₀ is selected from the group consisting of A, Aib, I, L, V, and W,
X₃₃ is selected from the group consisting of NMeQ, and Q,
X₃₄ is selected from the group consisting of ArgMe, bhArg, dOR, NMeR, and R,
X₃₅ is selected from the group consisting of Phe4F, Y, and NMeY;
wherein U is a lipid moiety of formula (II)

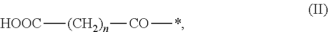

wherein n is an integer in the range 12 to 20,
wherein Z is a linker moiety comprising at least one [gGlu] residue,
wherein Z is linked to U on one end and to the epsilon-amino group of Lys at position-29 (Lys(29)) of the peptide P on the other end; and
wherein compound of general formula (I) comprises at least six (6) residues of amino acids selected from the group consisting of E and [gGlu];
or salt thereof, preferably a pharmaceutically acceptable salt thereof.

Emb. 2. The compound according to embodiment 1,
wherein Z is a linker moiety consisting of three (3) to ten (10) linker sub-moieties termed Z¹, Z², Z³, Z⁴, Z⁵, Z⁶, Z⁷, Z⁸, Z⁹, and Z¹⁰ covalently connected each other,
wherein the linker sub-moieties are independently selected from the group consisting of: absent, [gGlu], [eLys], [OEG], [AHX], [TRX], [gDab], and [dtOrn],
wherein Z comprises at least one [gGlu] residue (i.e. at least one (1) sub-moiety is [gGlu]), and
wherein the first linker sub-moieties of the linker Z (i.e. Z¹) is linked to U and the last linker sub-moiety on the other end of the linker Z is linked to the epsilon-amino group of Lys at position-29 of the peptide P.

Emb. 3. The compound according to any one of the preceding embodiments,
wherein P is a peptide comprising or, alternatively, consisting of the amino acid sequence,

(SEQ ID NO: 186)
Y-Aib-E-X₄-X₅-F-T-S-D-X₁₀-X₁₁-X₁₂-X₁₃-L-X₁₅-X₁₆-X₁₇-X₁₈-X₁₉-X₂₀-X₂₁-F-X₂₃-R-X₂₅-X₂₆-
X₂₇-X₂₈-K-X₃₀-T-R-X₃₃-X₃₄-X₃₅

wherein
X₄ is selected from the group consisting of A, and G,
X₅ is T,
X₁₀ is selected from the group consisting of F, L, and Y,
X₁₁ is selected from the group consisting of A, and S,
X₁₂ is I,
X₁₃ is selected from the group consisting of Aib, and aMeL,
X₁₅ is selected from the group consisting of D, and E,
X₁₆ is selected from the group consisting of K, N, and E,
X₁₇ is selected from the group consisting of A, and Q,
X₁₈ is selected from the group consisting of A, and R,
X₁₉ is selected from the group consisting of A, E, and Q,
X₂₀ is selected from the group consisting of A, Aib, and Y,
X₂₁ is selected from the group consisting of A, and S,
X₂₃ is selected from the group consisting of I, L, and V,
X₂₅ is selected from the group consisting of A, H and Y,
X₂₆ is L,
X₂₇ is selected from the group consisting of I, and L,
X₂₈ is N,
X₃₀ is selected from the group consisting of A, Aib, I, L, and V,
X₃₃ is selected from the group consisting of NMeQ, and Q,
X₃₄ is selected from the group consisting of dOR, and R,
X₃₅ is selected from the group consisting of Phe4F, Y, and NMeY.

Emb 4. The compound according to any one of the preceding embodiments,
wherein P is a peptide comprising or, alternatively, consisting of the amino acid sequence,

(SEQ ID NO: 187)
Y-Aib-E-X₄-X₅-F-T-S-D-X₁₀-X₁₁-X₁₂-X₁₃-L-X₁₅-X₁₆-X₁₇-X₁₈-X₁₉-X₂₀-X₂₁-F-X₂₃-R-X₂₅-
X₂₆-X₂₇-X₂₈-K-X₃₀-T-R-X₃₃-X₃₄-X₃₅

wherein
X₄ is selected from the group consisting of A, and G,
X₅ is T,
X₁₀ is selected from the group consisting of F, and Y,
X₁₁ is selected from the group consisting of A, and S,
X₁₂ is selected from the group consisting of A, E, and I,
X₁₃ is selected from the group consisting of Aib, and aMeL,
X₁₅ is selected from the group consisting of D, and E,
X₁₆ is selected from the group consisting of K, and N,
X₁₇ is Q,
X₁₈ is A,
X₁₉ is selected from the group consisting of A, E, and Q,
X₂₀ is selected from the group consisting of A, Aib, and Y,
X₂₁ is selected from the group consisting of A, and S,
X₂₃ is selected from the group consisting of I, L, and V,
X₂₅ is selected from the group consisting of H, and Y,
X₂₆ is L,
X₂₇ is selected from the group consisting of I, and L,
X₂₈ is N,
X₃₀ is selected from the group consisting of A, Aib, I, L, and V,
X₃₃ is Q,
X₃₄ is selected from the group consisting of dOR, and R,
X₃₅ is selected from the group consisting of Phe4F, and Y.

Emb. 5. The compound according to any one of the preceding embodiments,
wherein the C-terminal of peptide P is amidate (i.e. C-terminal is a primary amide, compounds of the invention are represented by formula U-Z-P-NH₂).

Emb. 6. The compound according to any one of the preceding embodiments,
wherein U is a lipid moiety of formula

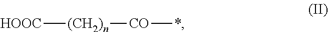

wherein n is an integer in the range 14 to 18.

Emb. 7. The compound according to any one of the preceding embodiments,
wherein U is selected from the group consisting of 15-carboxy-pentadecanoyl (briefly, C16DA), 17-carboxy-heptadecanoyl (briefly, C18DA), and 19-carboxy-nonadecanoyl (briefly, C20DA).

Emb. 8. The compound according to any one of the preceding embodiments,
wherein U is selected from the group consisting of 17-carboxy-heptadecanoyl (C18DA), and 19-carboxy-nonadecanoyl (C20DA).

Emb. 9. The compound according to any one of the preceding embodiments,
wherein U is 17-carboxy-heptadecanoyl (C18DA).

Emb. 10. The compound according to any one of the preceding embodiments,
wherein U is 19-carboxy-nonadecanoyl (C20DA).

Emb. 11. The compound according to any one of the preceding embodiments,
wherein Z is a linker moiety consisting of three (3) to eight (8) linker sub-moieties termed Z¹, Z², Z³, Z⁴, Z⁵, Z⁶, Z⁷, and Z⁸ covalently connected each other,
wherein the linker sub-moieties are independently selected from the group consisting of: absent, [gGlu], [eLys], [OEG], [AHX], [TRX], [gDab], and [dtOrn],
wherein Z comprises at least one [gGlu] residue, and
wherein the first linker sub-moieties Z¹ is linked to U and the last linker sub-moiety on the other end of the linker is linked to the epsilon-amino group of Lys at position-29 of the peptide P (e.g., U-Z¹-Z²-Z³-Z⁴-Z⁵-Z⁶-Z⁷-Z⁸-P).

Emb. 12. The compound according to any one of the preceding embodiments,
wherein U-Z is selected from the group consisting of:
U-[gGlu]-[OEG]-[OEG],
U-[gGlu]-[gGlu]-[gGlu]-[OEG]-[gGlu]-[gGlu]-[eLys], U-[gGlu]-[gGlu]-[gGlu]-[eLys]-[gGlu]-[gGlu]-[gGlu],
U-[gGlu]-[gGlu]-[gGlu]-[gGlu], U-[gGlu]-[gGlu]-[gGlu]-[gGlu]-[OEG], U-[gGlu]-[gGlu]-[gGlu]-[gGlu]-[OEG]-[OEG], U-[gGlu]-[gGlu]-[gGlu]-[gGlu]-[eLys], U-[gGlu]-[gGlu]-[gGlu]-[gGlu]-[AHX]-[gGlu]-[AHX], U-[gGlu]-[gGlu]-[gGlu]-[gGlu]-[OEG]-[gGlu]-[eLys], U-[gGlu]-[gGlu]-[gGlu]-[gGlu]-[OEG]-[gGlu]-[gGlu], U-[gGlu]-[gGlu]-[gGlu]-[gGlu]-[eLys]-[gGlu]-[gGlu],
U-[gGlu]-[gGlu]-[gGlu]-[gGlu]-[gGlu], U-[gGlu]-[gGlu]-[gGlu]-[gGlu]-[gGlu]-[OEG], U-[gGlu]-[gGlu]-[gGlu]-[gGlu]-[gGlu]-[OEG]-[OEG], U-[gGlu]-[gGlu]-[gGlu]-[gGlu]-[gGlu]-[eLys], U-[gGlu]-[gGlu]-[gGlu]-[gGlu]-[gGlu]-[eLys]-[eLys], U-[gGlu]-[gGlu]-[gGlu]-[gGlu]-[gGlu]-[AHX], U-[gGlu]-[gGlu]-[gGlu]-[gGlu]-[gGlu]-[AHX]-[AHX], U-[gGlu]-[gGlu]-[gGlu]-[gGlu]-[gGlu]-[OEG]-[eLys], U-[gGlu]-[gGlu]-[gGlu]-[gGlu]-[gGlu]-[eLys]-[gGlu],
U-[gGlu]-[gGlu]-[gGlu]-[gGlu]-[gGlu]-[gGlu], U-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[OEG], U-[gGlu]-[gGlu]-[gGlu]-[gGlu]-[gGlu]-[gGlu]-[eLys]-[eLys], U-[gGlu]-[gGlu]-[gGlu]-[gGlu]-[gGlu]-[gGlu]-[AHX], U-[gGlu]-[gGlu]-[gGlu]-[gGlu]-[gGlu]-[gGlu]-[gDab], U-[gGlu]-[gGlu]-[gGlu]-[gGlu]-[gGlu]-[gGlu]-[dtOrn], U-[gGlu]-[gGlu]-[gGlu]-[gGlu]-[gGlu]-[gGlu]-[gGlu], U-[gGlu]-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[gGlu]-[AHX],
U-[TRX]-[gGlu]-[gGlu]-[gGlu]-[gGlu]-[gGlu]-[gGlu], U-[TRX]-[gGlu]-[gGlu]-[gGlu]-[gGlu]-[gGlu]-[gGlu]-[eLys];
preferably, wherein U is 17-carboxy-heptadecanoyl (C18DA) or 19-carboxy-nonadecanoyl (C20DA).

Emb. 13. The compound according to any one of the preceding embodiments,
wherein Z comprises at least five (5) [gGlu] residues (i.e. at least five (5) sub-moieties are [gGlu]).

Emb. 14. The compound according to Emb. 11 and Emb. 13.

Emb. 15. The compound according to Emb. 14,
wherein U-Z is selected from the group consisting of:
U-[gGlu]-[gGlu]-[gGlu]-[OEG]-[gGlu]-[gGlu]-[eLys], U-[gGlu]-[gGlu]-[gGlu]-[eLys]-[gGlu]-[gGlu]-[gGlu],
U-[gGlu]-[gGlu]-[gGlu]-[gGlu]-[AHX]-[gGlu]-[AHX], U-[gGlu]-[gGlu]-[gGlu]-[gGlu]-[OEG]-[gGlu]-[eLys], U-[gGlu]-[gGlu]-[gGlu]-[gGlu]-[OEG]-[gGlu]-[gGlu], U-[gGlu]-[gGlu]-[gGlu]-[gGlu]-[eLys]-[gGlu]-[gGlu],
U-[gGlu]-[gGlu]-[gGlu]-[gGlu]-[gGlu], U-[gGlu]-[gGlu]-[gGlu]-[gGlu]-[gGlu]-[OEG], U-[gGlu]-[gGlu]-[gGlu]-[gGlu]-[gGlu]-[OEG]-[OEG], U-[gGlu]-[gGlu]-[gGlu]-[gGlu]-[gGlu]-[eLys], U-[gGlu]-[gGlu]-[gGlu]-[gGlu]-[gGlu]-[eLys]-[eLys], U-[gGlu]-[gGlu]-[gGlu]-[gGlu]-[gGlu]-[AHX], U-[gGlu]-[gGlu]-[gGlu]-[gGlu]-[gGlu]-[AHX]-[AHX], U-[gGlu]-[gGlu]-[gGlu]-[gGlu]-[gGlu]-[OEG]-[eLys], U-[gGlu]-[gGlu]-[gGlu]-[gGlu]-[gGlu]-[eLys]-[gGlu],
U-[gGlu]-[gGlu]-[gGlu]-[gGlu]-[gGlu]-[gGlu], U-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[OEG], U-[gGlu]-[gGlu]-[gGlu]-[gGlu]-[gGlu]-[gGlu]-[eLys]-[eLys], U-[gGlu]-[gGlu]-[gGlu]-[gGlu]-[gGlu]-[gGlu]-[AHX], U-[gGlu]-[gGlu]-[gGlu]-[gGlu]-[gGlu]-[gGlu]-[gDab], U-[gGlu]-[gGlu]-[gGlu]-[gGlu]-[gGlu]-[gGlu]-[dtOrn],
U-[gGlu]-[gGlu]-[gGlu]-[gGlu]-[gGlu]-[gGlu]-[gGlu], U-[gGlu]-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[gGlu]-[AHX],
U-[TRX]-[gGlu]-[gGlu]-[gGlu]-[gGlu]-[gGlu]-[gGlu], U-[TRX]-[gGlu]-[gGlu]-[gGlu]-[gGlu]-[gGlu]-[gGlu]-[eLys]; preferably wherein U is 17-carboxy-heptadecanoyl (C18DA) or 19-carboxy-nonadecanoyl (C20DA).

Emb. 16. The compound according to any one of the preceding embodiments,
wherein compound of formula (I) comprises at least seven (7) amino acids selected from the group consisting of E and [gGlu]; such as in the range 7 to 10 (i.e. 7, 8, 9, 10).

Emb. 17. The compound according to Emb. 14. and Emb. 16.

Emb. 18. The compound according to any one of the preceding embodiments,
wherein Z consists of seven (7) to eight (8) linker sub-moieties termed Z¹, Z², Z³, Z⁴, Z⁵, Z⁶, Z⁷, and Z⁸ covalently connected each other;
preferably wherein the linker sub-moieties are independently selected from the group consisting of absent, [gGlu], [eLys], [OEG], and [AHX].

Emb. 19. The compound according to Emb. 17 and Emb. 18.

Emb. 20. The compound according to Emb. 19,
wherein U-Z is selected from the group consisting of:
U-[gGlu]-[gGlu]-[gGlu]-[OEG]-[gGlu]-[gGlu]-[eLys],
U-[gGlu]-[gGlu]-[gGlu]-[gGlu]-[AHX]-[gGlu]-[AHX], U-[gGlu]-[gGlu]-[gGlu]-[gGlu]-[OEG]-[gGlu]-[gGlu], U-[gGlu]-[gGlu]-[gGlu]-[gGlu]-[gGlu]-[AHX]-[AHX], U-[gGlu]-[gGlu]-[gGlu]-[gGlu]-[gGlu]-[OEG]-[eLys], U-[gGlu]-[gGlu]-[gGlu]-[gGlu]-[gGlu]-[eLys]-[gGlu],
U-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[OEG], U-[gGlu]-[gGlu]-[gGlu]-[gGlu]-[gGlu]-[gGlu]-[AHX],
U-[gGlu]-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[gGlu]-[AHX];
preferably, wherein U is 17-carboxy-heptadecanoyl (C18DA) or 19-carboxy-nonadecanoyl (C20DA).

Emb. 21. The compound according to any one of the preceding embodiments, wherein X₁₃ is Aib.

Emb. 22. The compound according to any one of the preceding embodiments, wherein X₁₃ is aMeL.

Emb. 23. The compound according to any one of the preceding embodiments, wherein X₁₅ is E.

Emb. 24. The compound according to any one of the preceding embodiments, wherein X₁₅ is D.

Emb. 25. The compound according to any one of the preceding embodiments, wherein X₁₈ is A.

Emb. 26. The compound according to any one of the preceding embodiments, wherein X₁₈ is R.

Emb. 27. The compound according to any one of the preceding embodiments, wherein X₂₁ is A.

Emb. 28. The compound according to any one of the preceding embodiments, wherein X₂₁ is S.

Emb. 29. The compound according to any one of the preceding embodiments, wherein X₂₀ is Aib.

Emb. 30. The compound according to any one of the preceding embodiments, wherein X₃₀ is Aib.

Emb. 31. The compound according to any one of the preceding embodiments, wherein X₃₄ is R.

Emb. 32. The compound according to any one of the preceding embodiments, wherein X₃₅ is Y.

Emb. 33. The compound according to any one of the preceding embodiments,
wherein U-Z is C18DA-[gGlu]-[gGlu]-[gGlu]-[gGlu]-[gGlu]-[gGlu]-[eLys].

Emb. 34. The compound according to any one of the preceding embodiments,
wherein U-Z is C20DA-[gGlu]-[gGlu]-[gGlu]-[gGlu]-[gGlu]-[gGlu]-[eLys].

Emb. 35. The compound according to any one of the preceding embodiments,
wherein U-Z is C18DA-[gGlu]-[gGlu]-[gGlu]-[gGlu]-[gGlu]-[gGlu]-[OEG].

Emb. 36. The compound according to any one of the preceding embodiments,
wherein U-Z is C20DA-[gGlu]-[gGlu]-[gGlu]-[gGlu]-[gGlu]-[gGlu]-[OEG].

Emb. 37. The compound according to any one of the preceding embodiments,
wherein U-Z is C18DA-[gGlu]-[gGlu]-[gGlu]-[gGlu]-[gGlu]-[gGlu]-[AHX].

Emb. 38. The compound according to any one of the preceding embodiments,
wherein U-Z is C20DA-[gGlu]-[gGlu]-[gGlu]-[gGlu]-[gGlu]-[gGlu]-[AHX].

Emb. 39. The compound according to any one of the preceding embodiments,
wherein U-Z is C18DA-[gGlu]-[gGlu]-[gGlu]-[gGlu]-[gGlu]-[gGlu]-[gGlu]-[eLys].

Emb. 40. The compound according to any one of the preceding embodiments,
wherein U-Z is C20DA-[gGlu]-[gGlu]-[gGlu]-[gGlu]-[gGlu]-[gGlu]-[gGlu]-[eLys].

Emb. 41. The compound according to any one of the preceding embodiments,
wherein U-Z is C18DA-[gGlu]-[gGlu]-[gGlu]-[gGlu]-[gGlu]-[gGlu]-[gGlu]-[AHX].

Emb. 42. The compound according to any one of the preceding embodiments,
wherein U-Z is C20DA-[gGlu]-[gGlu]-[gGlu]-[gGlu]-[gGlu]-[gGlu]-[gGlu]-[AHX].

Emb. 43. The compound according to any one of the preceding embodiments,
wherein the compound is selected from the group consisting of Compound 1 (SEQ ID NO: 1) to Compound 176 (SEQ ID NO: 176) according to table 3 (table 3 incorporated by reference).

Emb. 44. The compound according to any one of the preceding embodiments,
wherein the compound bind to and/or activate GLP-1, GIP and NPY2 receptors, preferably the human receptors.

Emb. 45. The compound according to any one of the preceding embodiments,
wherein the compound shows/has a EC50 in the presence of human plasma (i.e. circa 100% hP) towards hGLP1R and hGIPR below 100 nM, and towards hNPY2R below 1000 nM;
preferably towards hGLP1R and hGIPR below or equal to 30 nM, and towards hNPY2R below 600 nM; more preferably towards hGLP1R and GIPR below or equal to 20 nM, and towards hNPY2R below 200 nM (as disclosed in Example 1).

Emb. 46. The compound according to any one of the preceding embodiments,
wherein the compound shows/has a solubility at or around physiological pH greater than or equal to (around) 1.0 mg/ml; more preferably greater or equal to than 3.0 mg/ml; even more preferably greater or equal to than 5.0 mg/ml (as disclosed in Example 2).

Emb. 47. The compound according to any one of the preceding embodiments,
wherein the compound has a half-life suitable for human therapy.

Emb. 48. The compound according to any one of the preceding embodiments,
wherein the compound has a half-life in mouse greater than 4 hours;
preferably greater than 6 hours; more preferably greater than 8 hours; even more preferably greater than 10 hours (as disclosed in Example 3).

Emb. 49. The compound according to
Emb. 3, Emb. 8 and Emb. 19; or Emb. 3, Emb. 9, and Emb. 20; or Emb. 3, Emb. 10, and Emb. 20; or Emb. 4, Emb. 9, and Emb. 20; or Emb. 4, Emb. 10, and Emb. 20; or Emb. 3, and any one of the embodiments selected from Emb. 33 to Emb. 42; or Emb. 4, and any one of the embodiments selected from Emb. 33 to Emb. 42; or Emb. 3, Emb. 21, and any one of the embodiments selected from Emb. 33 to Emb. 42; or Emb. 4, Emb. 22, and any one of the embodiments selected from Emb. 33 to Emb. 42; or Emb. 4, Emb. 9, Emb. 20, and Emb. 21; or Emb. 4, Emb. 10, Emb. 20, and Emb. 22; or Emb. 3, Emb. 8, Emb. 19, and Emb. 46; or Emb. 4, Emb. 8, Emb. 20, and Emb. 46.

### Biological Assays and Data

The following examples demonstrate certain specific embodiments of the present invention. The following examples were carried out using standard techniques that are well known and routine to those of skill in the art, except where otherwise described in detail.

### Example 1: HGLP1R/hGIPR/hNPY2R CreLuc Assay in 0.5% and 100% Plasma

CHO and HEK-293 recombinant cell lines express a luciferase reporter gene under control of the CAMP responsive element (CRE). As a second recombinant protein the GLP-1 and NPY2 receptors were expressed in the reporter gene HEK cells, and the GIP receptor was expressed in the reporter gene CHO cells, respectively. Stimulation of these recombinant cells with an agonist leads to an increase of intracellular CAMP levels. In the presence of CAMP, the transcription factor CRE binding protein (CREB) binds to the CRE and to the CREB binding protein (CBP), which leads to transcription of the luciferase reporter gene. Peptides upon serial-dilution in 100% DMSO were transferred into 384-well assay plates with 5 μl pre-dispensed assay buffer (1×HBSS, 20 mM HEPES, pH7.4 supplemented with 0.5% human plasma) using acoustic dispensing on a Labcyte ECHO. Cryo-preserved transgenic reporter gene cells were thawed in assay buffer. 20 μl (10.000 cells/well) were added to the plate with the peptides and incubated for 4 hours at 37° C. in a humidified incubator. Following incubation, assay plates are equilibrated to room temperature, followed by addition of 25 μl of Bright-Glo™ Luciferase reagent, incubation at room temperature for 10 minutes and analysis of luminescence (Envision Reader). Concentration response evaluation of compounds was performed with 8 concentrations of peptides (covering four decades). EC50 values were calculated by non-linear regression using sigmoid concentration-response with variable slope.

The same assays were run in the presence of 100% plasma with no additional buffer constituents.

The results are summarized in Table 1, below.

TABLE 1

| | hGLP1R | hGLP1R | hGIPR | hGIPR | hNPY2R | hNPY2R |
|---|---|---|---|---|---|---|
| Compound | 0.5% hP | 100% hP | 0.5% hP | 100% hP | 0.5% hP | 100% hP |
| No | (nM) | (nM) | (nM) | (nM) | (nM) | (nM) |
| 1 | 2.2 | 14.3 | 0.2 | 10.3 | 12.6 | 126.9 |
| 2 | 0.8 | 2.9 | 0.4 | 6.8 | 89.9 | 257.9 |
| 3 | 3.0 | 13.9 | 0.7 | 10.2 | 37.9 | 147.4 |
| 4 | 2.1 | 6.8 | 0.3 | 11.5 | 17.7 | 193.6 |
| 5 | 1.5 | 20.4 | 1.6 | 25.0 | 28.6 | 89.4 |
| 6 | 1.2 | 13.7 | 0.7 | 7.3 | 14.7 | 51.0 |
| 7 | 0.7 | 7.9 | 0.4 | 10.6 | 12.4 | 80.6 |
| 8 | 0.3 | 4.2 | 0.3 | 14.2 | 40.6 | 475.8 |
| 9 | 0.5 | 2.4 | 0.1 | 4.9 | 25.1 | 75.4 |
| 10 | 1.2 | 1.2 | 0.6 | 2.4 | 26.3 | 28.2 |
| 11 | 1.5 | 4.3 | 0.3 | 8.0 | 9.5 | 64.7 |
| 12 | 1.8 | 7.7 | 0.8 | 23.4 | 47.6 | 140.4 |
| 13 | 0.4 | 4.2 | 0.1 | 5.1 | 18.7 | 121.3 |
| 14 | 0.3 | 0.9 | 0.1 | 6.2 | 16.8 | 387.5 |
| 15 | 15.7 | 7.5 | 0.6 | 7.8 | 8.1 | 59.8 |
| 16 | 0.7 | 1.8 | 0.5 | 2.0 | 6.4 | 59.1 |
| 17 | 1.3 | 3.2 | 0.2 | 2.0 | 29.4 | 60.9 |
| 18 | 2.4 | 8.4 | 0.2 | 6.8 | 15.1 | 101.4 |
| 19 | 2.7 | 13.0 | 0.9 | 51.5 | 10.0 | 28.2 |
| 20 | 3.9 | 11.0 | 0.4 | 6.9 | 9.5 | 75.4 |
| 21 | 4.8 | 13.9 | 0.2 | 5.6 | 6.5 | 110.7 |
| 22 | 0.2 | 0.6 | 0.1 | 1.5 | 8.8 | 72.0 |
| 23 | 0.5 | 5.1 | 0.2 | 3.1 | 12.5 | 49.2 |
| 24 | 0.2 | 2.9 | 1.0 | 26.2 | 45.0 | 209.0 |
| 25 | 0.7 | 2.0 | 0.4 | 7.6 | 89.2 | 437.4 |
| 26 | 3.5 | 7.7 | 0.3 | 5.6 | 21.4 | 215.7 |
| 27 | 3.6 | 20.8 | 0.6 | 16.3 | 5.2 | 69.1 |
| 28 | 8.0 | 14.8 | 19.3 | 207.5 | 24.0 | 296.8 |
| 29 | 5.5 | 33.0 | 0.8 | 14.1 | 41.3 | 179.5 |
| 30 | 1.1 | 0.9 | 0.3 | 3.5 | 33.9 | 81.1 |
| 31 | 3.2 | 15.4 | 0.6 | 14.9 | 76.4 | 131.3 |
| 32 | 1.1 | 3.1 | 0.5 | 6.5 | 17.1 | 44.6 |
| 33 | 0.1 | 1.0 | 0.7 | 18.7 | 27.1 | 169.0 |
| 34 | 0.3 | 2.5 | 0.2 | 3.5 | 14.6 | 28.0 |
| 35 | 3.5 | 6.6 | 0.7 | 10.9 | 22.9 | 98.5 |
| 36 | 5.2 | 16.9 | 8.8 | 41.4 | 30.1 | 95.2 |
| 37 | 5.8 | 9.1 | 2.9 | 15.5 | 11.9 | 41.3 |
| 38 | 1.5 | 11.8 | 0.6 | 10.7 | 104.8 | 140.7 |
| 39 | 2.2 | 10.4 | 0.9 | 22.9 | 10.8 | 119.4 |
| 40 | 0.3 | 3.5 | 0.3 | 3.7 | 16.2 | 59.4 |
| 41 | 0.9 | 5.0 | 0.3 | 7.1 | 49.2 | 71.3 |
| 42 | 0.9 | 14.5 | 0.2 | 13.0 | 81.4 | 704.1 |
| 43 | 0.6 | 3.0 | 0.1 | 2.8 | 49.3 | 189.3 |
| 44 | 0.2 | 2.4 | 0.1 | 2.3 | 6.9 | 59.3 |
| 45 | 0.5 | 3.6 | 0.3 | 19.6 | 19.8 | 224.0 |
| 46 | 5.7 | 5.3 | 0.8 | 7.2 | 28.4 | 70.5 |
| 47 | 0.8 | 3.1 | 0.3 | 6.2 | 19.9 | 72.6 |
| 48 | 0.5 | 6.2 | 0.1 | 2.5 | 11.6 | 77.2 |
| 49 | 0.3 | 1.1 | 0.1 | 7.6 | 62.7 | 763.2 |
| 50 | 0.7 | 6.3 | 0.7 | 23.3 | 334.6 | 522.5 |
| 51 | 4.5 | 16.3 | 0.5 | 13.8 | 56.5 | 101.3 |
| 52 | 3.3 | 5.8 | 0.3 | 7.4 | 14.3 | 152.4 |
| 53 | 0.3 | 1.1 | 0.1 | 1.7 | 8.2 | 81.2 |
| 54 | 3.4 | 14.4 | 2.6 | 64.5 | 8.6 | 38.3 |
| 55 | 0.4 | 3.1 | 0.4 | 7.0 | 18.3 | 107.6 |
| 56 | 0.7 | 7.1 | 0.4 | 19.3 | 122.1 | 748.5 |
| 57 | 1.1 | 6.3 | 0.3 | 16.1 | 68.9 | 715.0 |
| 58 | 7.8 | 56.3 | 2.7 | 27.4 | 11.4 | 40.0 |
| 59 | 2.5 | 16.7 | 0.4 | 7.4 | 5.7 | 66.2 |
| 60 | 4.4 | 12.3 | 0.3 | 8.0 | 12.3 | 124.2 |
| 61 | 4.1 | 10.9 | 0.4 | 2.9 | 24.1 | 42.1 |
| 62 | 0.4 | 2.9 | 0.3 | 4.9 | 37.3 | 71.7 |
| 63 | 0.4 | 4.2 | 0.2 | 7.7 | 72.1 | 348.0 |
| 64 | 0.5 | 1.5 | 0.5 | 8.2 | 161.6 | 720.5 |
| 65 | 4.9 | 20.2 | 0.7 | 16.8 | 65.8 | 406.0 |
| 66 | 2.3 | 5.4 | 0.2 | 6.9 | 9.8 | 99.3 |
| 67 | 0.1 | 0.6 | 0.1 | 1.7 | 18.1 | 112.2 |
| 68 | 0.6 | 3.3 | 0.2 | 3.0 | 150.4 | 690.2 |
| 69 | 0.7 | 2.9 | 0.2 | 5.5 | 7.0 | 56.0 |
| 70 | 0.2 | 5.2 | 0.2 | 8.1 | 12.8 | 119.1 |
| 71 | 3.7 | 12.3 | 0.3 | 2.1 | 45.0 | 150.6 |
| 72 | 0.6 | 1.4 | 0.2 | 1.1 | 73.7 | 171.0 |
| 73 | 0.4 | 1.4 | 0.4 | 5.9 | 23.4 | 135.9 |
| 74 | 0.4 | 3.3 | 0.5 | 8.2 | 7.1 | 90.6 |
| 75 | 1.4 | 24.5 | 0.3 | 13.2 | 190.6 | 904.1 |
| 76 | 0.0 | 0.3 | 0.0 | 0.5 | 7.2 | 24.3 |
| 77 | 0.4 | 1.8 | 0.3 | 3.7 | 32.7 | 58.7 |
| 78 | 0.2 | 1.1 | 0.1 | 2.1 | 11.4 | 56.7 |
| 79 | 0.2 | 1.9 | 0.3 | 4.7 | 57.9 | 99.1 |
| 80 | 3.5 | 2.9 | 1.7 | 8.3 | 12.0 | 34.0 |
| 81 | 0.6 | 3.8 | 0.5 | 6.3 | 7.2 | 52.4 |
| 82 | 4.2 | 3.3 | 1.2 | 11.7 | 6.4 | 75.1 |
| 83 | 0.6 | 1.4 | 0.4 | 3.7 | 16.8 | 31.7 |
| 84 | 10.6 | 57.6 | 5.0 | 48.4 | 18.5 | 55.0 |
| 85 | 10.4 | 14.5 | 4.9 | 22.3 | 8.3 | 16.2 |
| 86 | 0.8 | 3.7 | 0.3 | 5.9 | 33.2 | 162.3 |
| 87 | 1.7 | 5.6 | 0.2 | 4.9 | 16.5 | 57.9 |
| 88 | 0.2 | 1.8 | 0.1 | 1.7 | 7.4 | 30.9 |
| 89 | 0.2 | 1.7 | 0.1 | 0.9 | 19.7 | 159.8 |
| 90 | 2.0 | 1.5 | 0.2 | 0.6 | 10.6 | 27.1 |
| 91 | — | 13.7 | 0.8 | 14.4 | 9.7 | 45.2 |
| 92 | 0.4 | 2.0 | 0.2 | 2.5 | 9.9 | 22.6 |
| 93 | 1.2 | 1.9 | 1.6 | 17.6 | 9.4 | 24.8 |
| 94 | 2.9 | 9.2 | 0.3 | 6.3 | 9.4 | 100.9 |
| 95 | 10.5 | 43.5 | 1.5 | 9.3 | 58.0 | 96.3 |
| 96 | 0.9 | 1.7 | 0.3 | 1.7 | 23.0 | 125.4 |
| 97 | 3.8 | 16.8 | 0.6 | 12.7 | 11.7 | 134.9 |
| 98 | 0.7 | 2.4 | 0.5 | 7.8 | 27.3 | 202.2 |
| 99 | 4.1 | 9.5 | 0.5 | 9.0 | 16.7 | 124.8 |
| 100 | 0.2 | 1.9 | 0.5 | 14.3 | 101.7 | 407.0 |
| 101 | 4.6 | 20.0 | 1.0 | 21.0 | 98.0 | 364.2 |
| 102 | 0.1 | 1.2 | 0.1 | 2.8 | 7.8 | 26.0 |
| 103 | 0.4 | 0.6 | 0.2 | 3.6 | 25.9 | 130.8 |
| 104 | 2.0 | 12.9 | 1.2 | 14.0 | 17.5 | 129.3 |
| 105 | 0.3 | 3.4 | 0.4 | 6.6 | 19.9 | 49.6 |
| 106 | 1.1 | 6.1 | 0.2 | 4.7 | 7.1 | 41.7 |
| 107 | 1.4 | 3.3 | 0.7 | 8.4 | 13.3 | 20.3 |
| 108 | 0.3 | 5.1 | 0.2 | 6.8 | 20.3 | 119.7 |
| 109 | 0.8 | 5.1 | 0.3 | 4.2 | 6.6 | 78.2 |
| 110 | 0.7 | 2.3 | 0.2 | 3.8 | 14.2 | 51.4 |
| 111 | 4.7 | 6.1 | 0.3 | 2.2 | 53.6 | 77.4 |
| 112 | 6.6 | 31.6 | 0.5 | 10.3 | 13.7 | 27.9 |
| 113 | 0.1 | 1.2 | 0.2 | 5.5 | 15.4 | 100.5 |
| 114 | 1.8 | 10.0 | 0.5 | 13.1 | 82.1 | 657.3 |
| 115 | 7.0 | 9.8 | 1.4 | 22.2 | 10.1 | 107.3 |
| 116 | 2.0 | 2.7 | 0.3 | 3.4 | 92.8 | 207.7 |
| 117 | 0.4 | 2.8 | 0.2 | 3.6 | 8.8 | 39.2 |
| 118 | 6.4 | 12.5 | 0.3 | 2.5 | 11.0 | 55.9 |
| 119 | 0.1 | 0.4 | 0.1 | 0.8 | 10.9 | 43.7 |
| 120 | 1.7 | 4.0 | 0.3 | 4.6 | 23.9 | 56.4 |
| 121 | 0.4 | 2.1 | 0.2 | 2.8 | 13.8 | 33.0 |
| 122 | 0.3 | 2.2 | 0.1 | 4.5 | 60.8 | 284.2 |
| 123 | 6.7 | 15.7 | 0.3 | 6.8 | 51.6 | 231.7 |
| 124 | 0.5 | 8.1 | 0.1 | 4.0 | 8.2 | 54.4 |
| 125 | 1.1 | 6.1 | 0.4 | 5.2 | 6.4 | 60.8 |
| 126 | 4.3 | 14.3 | 0.4 | 10.5 | 12.5 | 116.1 |
| 127 | 2.4 | 4.0 | 0.4 | 16.7 | 10.3 | 76.9 |
| 128 | 0.7 | 4.0 | 0.2 | 5.7 | 116.0 | 232.9 |
| 129 | 0.9 | 2.8 | 0.4 | 7.1 | 212.8 | 827.3 |
| 130 | 0.5 | 2.3 | 0.5 | 10.0 | 75.0 | 788.8 |
| 131 | 0.1 | 1.2 | 0.3 | 6.8 | 17.5 | 85.9 |
| 132 | 5.4 | 4.4 | 0.4 | 7.6 | 21.4 | 170.6 |
| 133 | 2.7 | 9.7 | 0.3 | 3.4 | 44.9 | 245.9 |
| 134 | 1.0 | 4.0 | 0.4 | 8.2 | 32.8 | 34.7 |
| 135 | 6.3 | 7.4 | 0.5 | 5.8 | 18.8 | 47.8 |
| 136 | 2.7 | 8.1 | 0.5 | 9.1 | 38.5 | 123.1 |
| 137 | 3.9 | 13.7 | 0.5 | 11.5 | 20.1 | 46.4 |
| 138 | 1.2 | 9.0 | 0.5 | 11.5 | 13.4 | 160.3 |
| 139 | 17.9 | 23.4 | 12.2 | 22.3 | 12.3 | 22.7 |
| 140 | 1.1 | 3.5 | 0.2 | 8.0 | 22.0 | 140.6 |
| 141 | 0.8 | 4.1 | 0.1 | 6.3 | 9.4 | 99.0 |
| 142 | 2.6 | 8.0 | 0.3 | 3.4 | 17.4 | 31.8 |
| 143 | 1.2 | 3.1 | 0.3 | 2.6 | 35.4 | 48.7 |
| 144 | 1.9 | 9.4 | 0.4 | 4.8 | 21.0 | 72.4 |
| 145 | 0.5 | 1.8 | 0.3 | 5.4 | 40.7 | 94.2 |
| 146 | 1.0 | 2.1 | 0.2 | 3.9 | 51.5 | 89.8 |
| 147 | 0.3 | 2.3 | 0.2 | 5.0 | 11.1 | 86.3 |
| 148 | 3.2 | 3.9 | 0.8 | 17.3 | 9.5 | 68.0 |
| 149 | 2.1 | 3.3 | 0.6 | 7.6 | 24.5 | 165.5 |
| 150 | 0.5 | 1.7 | 0.1 | 2.8 | 9.8 | 90.7 |
| 151 | 3.9 | 7.0 | 0.5 | 5.6 | 32.6 | 89.8 |
| 152 | 1.1 | 2.9 | 0.5 | 13.0 | 21.7 | 160.0 |
| 153 | 0.1 | 1.5 | 0.1 | 1.5 | 40.0 | 65.5 |
| 154 | 0.5 | 4.3 | 0.1 | 3.6 | 34.4 | 122.1 |
| 155 | 1.1 | 4.7 | 0.7 | 6.1 | 6.0 | 63.1 |
| 156 | 7.0 | 33.1 | 0.8 | 14.8 | 16.3 | 44.1 |
| 157 | 1.6 | 6.4 | 0.2 | 6.1 | 15.9 | 134.8 |
| 158 | 0.6 | 1.9 | 0.5 | 14.4 | 26.0 | 149.2 |
| 159 | 1.3 | 2.9 | 0.2 | 6.7 | 11.5 | 57.5 |
| 160 | 7.1 | 27.0 | 0.4 | 3.5 | 15.5 | 28.5 |
| 161 | 2.0 | 5.0 | 1.1 | 14.1 | 7.7 | 72.8 |
| 162 | 2.9 | 3.7 | 0.7 | 14.0 | 11.8 | 43.8 |
| 163 | 0.1 | 2.2 | 0.2 | 5.9 | 14.3 | 218.4 |
| 164 | 0.6 | 2.2 | 0.4 | 3.5 | 22.8 | 56.9 |
| 165 | 3.7 | 10.7 | 0.3 | 3.6 | 17.1 | 32.9 |
| 166 | 8.0 | 27.4 | 0.6 | 15.4 | 28.8 | 279.4 |
| 167 | 1.0 | 18.7 | 0.2 | 10.5 | 13.3 | 267.1 |
| 168 | 1.1 | 2.7 | 0.2 | 4.6 | 27.7 | 69.7 |
| 169 | 2.6 | 6.3 | 0.4 | 4.4 | 17.9 | 34.0 |
| 170 | 2.6 | 9.8 | 0.4 | 7.4 | 43.8 | 244.5 |
| 171 | 4.2 | 24.2 | 0.4 | 6.7 | 9.9 | 95.3 |
| 172 | 4.1 | 13.1 | 1.7 | 19.8 | 52.7 | 136.1 |
| 173 | 0.2 | 2.5 | 0.7 | 20.1 | 7.3 | 55.5 |
| 174 | 1.0 | 4.1 | 0.2 | 4.6 | 11.4 | 40.6 |
| 175 | 2.2 | 8.6 | 0.2 | 5.6 | 7.5 | 33.8 |
| 176 | 2.5 | 12.6 | 0.6 | 27.1 | 32.8 | 457.2 |

### Example 2: Solubility

Peptides (as TFA-salts) were weighed out in a filter unit (Mini-UniPrep Syringeless Filter 0.45 μm, Whatman PVDF), and 0.1 M phosphate buffer at pH 7 was added to achieve 10 mg/mL final peptide concentration. The peptides were dissolved by shaking the filter units horizontally at 600 rpm for 2 hours at room temperature. Samples were then filtered to remove any insoluble particles. Controls were prepared by weighing out the corresponding peptide solid material and dissolving it in an appropriate vehicle (e.g. ACN:H₂O) to a final concentration of 1 mg/mL. Both, the control and sample were analyzed by reversed phase chromatography. The area under the peak of the sample was compared to the control and the solubility was calculated based on that ratio. The pH was measured and recorded for each sample.

Solubility of compounds according to the invention and reference compounds have been measured and summarized in table 2 below.

TABLE 2

| Compound No | Solubility (mg/ml) | pH |
|---|---|---|
| 1 | 9.8 | 6.8 |
| 2 | 5 | 6.8 |
| 3 | 6.9 | 6.8 |
| 4 | 10 | 6.8 |
| 6 | 8.9 | 6.7 |
| 7 | 4.5 | 6.8 |
| 8 | 4.9 | 6.8 |
| 9 | 4.7 | 6.8 |
| 10 | 3.8 | 6.8 |
| 11 | 0.8 | 6.8 |
| 12 | 5 | 6.8 |
| 13 | 4.3 | 6.8 |
| 14 | 4.8 | 6.8 |
| 15 | 9.6 | 6.8 |
| 17 | 4 | 6.8 |
| 18 | 4.7 | 6.8 |
| 19 | 0 | 6.8 |
| 20 | 8.5 | 6.8 |
| 23 | 9.4 | 6.7 |
| 24 | 8.1 | 6.7 |
| 25 | 4.8 | 6.8 |
| 26 | 9.4 | 6.7 |
| 27 | 10 | 6.8 |
| 28 | 10 | 6.7 |
| 29 | 10 | 6.6 |
| 30 | 4.7 | 6.8 |
| 31 | 4.3 | 6.8 |
| 32 | 9.1 | 6.7 |
| 33 | 8.9 | 6.7 |
| 34 | 10.6 | 6.8 |
| 35 | 5.2 | 6.8 |
| 36 | 5 | 6.8 |
| 37 | 9 | 6.6 |
| 38 | 4.3 | 6.8 |
| 39 | 4.5 | 6.8 |
| 40 | 9.5 | 6.7 |
| 41 | 4 | 6.8 |
| 42 | 4.3 | 6.8 |
| 43 | 4.3 | 6.8 |
| 44 | 9.8 | 6.8 |
| 45 | 5 | 6.8 |
| 46 | 4.2 | 6.8 |
| 47 | 10 | 6.8 |
| 48 | 7.7 | 6.7 |
| 49 | 10 | 6.8 |
| 50 | 4.5 | 6.8 |
| 51 | 9.3 | 6.8 |
| 52 | 9.4 | 6.8 |
| 53 | 10 | 7.0 |
| 54 | 9.6 | 6.7 |
| 55 | 8.8 | 6.7 |
| 56 | 4.4 | 6.8 |
| 57 | 10 | 6.7 |
| 58 | 3.3 | 6.6 |
| 59 | 2.1 | 6.9 |
| 60 | 9.6 | 6.8 |
| 61 | 3.7 | 6.8 |
| 62 | 4.1 | 6.8 |
| 63 | 4.6 | 6.8 |
| 64 | 4.8 | 6.8 |
| 65 | 3.9 | 6.8 |
| 66 | 5 | 6.8 |
| 67 | 8.8 | 6.7 |
| 68 | 4.8 | 6.5 |
| 69 | 4.8 | 6.8 |
| 70 | 9.1 | 6.7 |
| 71 | 4.7 | 6.8 |
| 72 | 3.8 | 6.8 |
| 73 | 4.9 | 6.9 |
| 74 | 8.6 | 6.7 |
| 75 | 4.3 | 6.8 |
| 76 | 8.9 | 6.7 |
| 77 | 0.4 | 6.8 |
| 78 | 10.3 | 6.7 |
| 79 | 4.2 | 6.8 |
| 82 | 9 | 6.8 |
| 83 | 3.8 | 6.8 |
| 84 | 4.1 | 6.8 |
| 85 | 9.4 | 6.8 |
| 87 | 4.9 | 6.8 |
| 88 | 10.4 | 6.6 |
| 89 | 8.8 | 6.8 |
| 91 | 2.2 | 6.8 |
| 92 | 6.7 | 6.8 |
| 93 | 10 | 6.8 |
| 94 | 9 | 6.7 |
| 95 | 4.4 | 6.8 |
| 97 | 4.7 | 6.8 |
| 98 | 4.9 | 6.9 |
| 99 | 10 | 6.8 |
| 100 | 5.3 | 6.7 |
| 101 | 5.2 | 6.8 |
| 102 | 9.1 | 6.7 |
| 103 | 9.7 | 6.8 |
| 104 | 9.4 | 6.6 |
| 105 | 8 | 6.7 |
| 106 | 9.2 | 6.8 |
| 107 | 3.9 | 6.8 |
| 108 | 9.2 | 6.6 |
| 109 | 10 | 6.8 |
| 110 | 0.8 | 6.9 |
| 111 | 4 | 6.8 |
| 112 | 4.4 | 6.8 |
| 113 | 8.7 | 6.7 |
| 115 | 8.2 | 6.9 |
| 116 | 4.2 | 6.8 |
| 117 | 4.8 | 6.9 |
| 118 | 4.1 | 6.8 |
| 119 | 9 | 7.0 |
| 120 | 4.7 | 6.8 |
| 121 | 4.2 | 6.8 |
| 122 | 3.8 | 6.8 |
| 123 | 4.3 | 6.8 |
| 124 | 10 | 6.8 |
| 125 | 6.7 | 6.8 |
| 126 | 10 | 6.8 |
| 127 | 4.5 | 6.8 |
| 128 | 4.1 | 6.8 |
| 129 | 5.1 | 6.8 |
| 130 | 5 | 6.8 |
| 131 | 9 | 6.6 |
| 132 | 9.2 | 6.8 |
| 133 | 4.2 | 6.8 |
| 135 | 9.2 | 6.7 |
| 136 | 9.6 | 6.8 |
| 137 | 4.3 | 6.8 |
| 138 | 9.3 | 6.7 |
| 139 | 9 | 6.6 |
| 140 | 4.9 | 6.8 |
| 141 | 1.1 | 6.8 |
| 143 | 2.8 | 6.8 |
| 144 | 4.3 | 6.8 |
| 145 | 5 | 6.8 |
| 146 | 5 | 6.8 |
| 148 | 8.6 | 6.8 |
| 149 | 4.9 | 6.8 |
| 150 | 4.8 | 6.8 |
| 151 | 10.3 | 6.8 |
| 152 | 4.2 | 6.8 |
| 153 | 8.2 | 6.7 |
| 154 | 4.4 | 6.8 |
| 155 | 8.9 | 6.8 |
| 156 | 5.1 | 6.8 |
| 157 | 5.2 | 6.8 |
| 158 | 3.2 | 6.8 |
| 159 | 9.6 | 6.8 |
| 160 | 5.2 | 6.8 |
| 161 | 10 | 6.8 |
| 162 | 8.7 | 6.8 |
| 163 | 5 | 6.8 |
| 164 | 4.5 | 6.8 |
| 165 | 5 | 6.8 |
| 167 | 10 | 6.7 |
| 168 | 8.2 | 6.8 |
| 169 | 9.4 | 6.8 |
| 170 | 4.6 | 6.8 |
| 172 | 3.1 | 6.6 |
| 174 | 9.1 | 6.8 |
| 175 | 8.4 | 6.8 |
| 176 | 5.3 | 6.8 |
| Ref. 1 | <1 | 6.8 |
| Ref. 2 | <1 | 6.8 |
| Ref. 3 | <1 | 6.8 |

### Example 3: Mouse PK

Pharmacokinetic parameters of the peptides were determined after intravenous application to NMRI mice (Naval Medical Research Institute mice). Male NMRI mice were obtained from Janvier Laboratories (France) weighing 30 g to 40 g. Mice were housed in standard cages with a 12:12 hours light:dark cycle and standard food and water access ad libitum. Each test peptide was dissolved in 50 mM phosphate buffer (pH 7.4)/3.5% mannitol. Intravenous application of 30-60 nmol/kg was performed via the tail vein. Serial blood samples from conscious mice were collected from the saphenous vein in EDTA-containing vials at different time points up to 56 hours post-dosing. Subsequently, plasma was prepared by centrifugation for 5 minutes at approximately 5000 rpm and stored at −20° C. until quantification of the peptide plasma levels by liquid chromatography mass spectrometry (LC-MS). Individual plasma concentration-time profiles were analysed by a non-compartmental approach and the resulting pharmacokinetic parameters were calculated. Mouse mean residence times (MRT) of GGY peptides according to the invention have been measured and summarized in table 3 below.

TABLE 3

| Compound No | Mouse MRT (hours) |
|---|---|
| 3 | 17.2 |
| 9 | 7.5 |
| 10 | 7.3 |
| 53 | 14.4 |
| 62 | 8.9 |
| 69 | 4.6 |
| 72 | 9.6 |
| 74 | 16.4 |
| 76 | 6.7 |
| 77 | 7.4 |
| 79 | 7 |
| 83 | 7.8 |
| 92 | 7.5 |
| 106 | 12.8 |
| 109 | 13.8 |
| 119 | 9.6 |
| 121 | 10 |
| 125 | 21.2 |
| 134 | 5.3 |
| 150 | 15.9 |

### Example 4: Effect on Acute Food Intake in Normal NMRI Mice

Male NMRI mice were obtained from Janvier (Janvier Labs, France) or from Charles River (Charles River Research Models & Services Germany GmbH) at the age of 3 weeks. The animals get a microchip (Datamars, Slim Microchip T-SL) for individual identification after delivery. They were housed with 4 mice per cage under a 12/12 dark-light cycle, light off at 3 p.m. Room temperature was controlled to 21° C.+/−1° C. and humidity 60%+/−20%. Mice had ad libitum access to regular rodent chow (KLIBA Nafag 3040 or Altromin 1324, Brogaarden, Denmark) and tap water.

Latest transfer into the HM2-System of MBRose, Denmark (real-time monitoring system of food- and water intake) of the animals 5 days before study start, to allow acclimatization to experimental conditions. As the animals were uniquely identified with the microchips, each individual animal was identified by its own microchip upon entry and exit from the food channel via antennae. Randomization of the mice for each study group (n=8; age earliest 6 weeks old) was based on food intake (middle of last three days 24 h-interval) and bodyweight directly before start of the study. A vehicle treated (50 mM phosphate buffer pH 7.4 with 3.5% mannitol) group was included in each experiment. To have the same nutrition standard for each animal the food access was locked eight hours before dark phase. One hour before night, the animals were once treated subcutaneously with the test peptide. Food intake was reported hourly for a period of 24 hours. The food intake was normalized [%] to the average food intake of the vehicle group and the value are summarized in table 4 below.

TABLE 4

| | Acute Food Intake 24 hours |
|---|---|
| Compound No | 100 nmol/kg [% CTL] |
| 3 | 74 |
| 9 | 56 |
| 20 | 79 |
| 53 | 47 |
| 76 | 55 |
| 78 | 65 |
| 88 | 47 |
| 92 | 58 |
| 106 | 60 |
| 119 | 34 |
| 125 | 55 |
| 150 | 46 |

### General Procedure for Solid Phase Synthesis of Peptide

All peptides were synthesized by standard Fmoc-based solid phase peptide chemistry on a Tentagel S RAM resin (loading 0.23-0.25 mmol/g, bead size 90 μm) supplied by Iris Biotech GmbH or Rapp Polymere GmbH.

The following protected amino acids were used: Fmoc-Ala-OH, Fmoc-Aib-OH, Fmoc-Arg(Pbf)-OH, Fmoc-ArgMe(Pbf)-OH, Fmoc-NMeR(Pbf)-OH, Fmoc-bhArg(Pbf)-OH, Fmoc-Asn(Trt)-OH, Fmoc-Asp(tBu)-OH, Fmoc-Gln(Trt)-OH, Fmoc-NMeQ(Trt)-OH, Fmoc-Glu(tBu)-OH, Fmoc-Glu-OtBu, Fmoc-Gly-OH, Fmoc-His(Trt)-OH, Fmoc-Ile-OH, Fmoc-aMeL-OH, Fmoc-Leu-OH, Fmoc-Lys(Boc)-OH, Fmoc-Lys(Dde)-OH, Fmoc-Lys(Mtt)-OH, Fmoc-Phe-OH, Fmoc-Phe4F-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-NMeY(tBu)-OH, Boc-Tyr(tBu)-OH, Fmoc-D-Tyr(tBu)-OH, Fmoc-Val-OH. The L-form of the amino acid building blocks was utilized if not specified otherwise.

The modular half-life extending group was built up by solid-phase peptide synthesis (SPPS) using protected building blocks such as, but not limited to, C18DA(tBu), C20DA(tBu), Fmoc-OEG-OEG-OH, Fmoc-OEG-OH, Boc-Lys(Fmoc)-OH, Fmoc-Glu-OtBu, Fmoc-Glu(tBu)-OH, Fmoc-AHX-OH, Fmoc-TRX-OH, Boc-ORN(Fmoc)-OH and Boc-DAB(Fmoc)-OH.

The amino acids, Fmoc-Glu-OtBu, Oxyma and DIC, were purchased from standard suppliers, e.g. Bachem, Novabiochem, ABCR GmbH & CO. KG., Iris Biotech GmbH, Sigma-Aldrich. C18DA(tBu) and C20DA(tBu) were supplied by Cool Pharm Ltd. or AstraTech, Fmoc-OEG-OEG-OH was supplied by ABCR GmbH & CO. KG or Iris Biotech GmbH, Fmoc-OEG-OH was supplied by Angene International Limited, Combi Blocks Inc., Iris Biotech GmbH or Hangzhou APIChem Technology Co., Ltd. Fmoc-AHX-OH was purchased from Activate Scientific GmbH, Fmoc-TRX-OH was purchased from ABCR GmbH & CO. KG.

Assembly of peptides started from the C-terminus by stepwise chain elongation towards the N-terminus according to the respective sequences until the N-terminal amino acid was reached. Deprotection of the side chain of the branching amino acid, e.g. Lys(Dde), was followed by assembly of the half-life extending group.

The peptides were obtained as TFA salts from cleavage/deprotection or from HPLC purification. The trifluoroacetate can be exchanged by common procedures, such as resin-ion exchange procedures, e.g. as disclosed in Roux, St. et al. J. Pept. Sci. 2008; 14:354-359.

### Synthesis Method 1 (S01)

Peptides were synthesized by microwave-assisted solid-phase peptide synthesis (SPPS) on a CEM Liberty Blue Peptide Synthesizer at 0.25 mmol scale on Tentagel S RAM resin using the Fmoc strategy. The stoichiometry and concentrations for peptide coupling reactions were 4 eq of suitably protected amino acid in DMF (0.2 mol/l, 5 ml), 4 eq of Oxyma in DMF (1 mol/l, 1 ml) and 8 eq DIC in DMF (1 mol/l, 2 ml).

The reaction times and temperatures were different for the amino acids. Single coupling 4 minutes (i.e. min) at 90° C. was used for all standard amino acids except those mentioned below. Single coupling 15 min at 75° C. was used for Fmoc-Glu-OtBu. Double coupling 4 min at 90° C. was used for Fmoc-Arg(Pbf)-OH, C18DA(tBu), C20DA(tBu), Fmoc-OEG-OH, Boc-Lys(Fmoc)-OH, Fmoc-NMeR(Pbf)-OH, Fmoc-NMeQ(Trt)-OH and Fmoc-AHX-OH. The amino acid after FMOC-Aib-OH, NMeR and NMeQ was coupled twice. Fmoc-aMeL-OH, the first 8 amino acids and the last 10 amino acids in the main chain were coupled at 90° C. for 8 min.

A capping step with 20% acetic anhydride in DMF (10 ml) for 5 min at 65° C. was performed prior to the coupling of Fmoc-Glu-OtBu and prior to the coupling of C18DA(tBu) and C20DA(tBu). N° Fmoc deprotection was performed with 10% piperidine/DMF (10 ml) for 1 min at 90° C. and resin was deprotected two times prior to coupling the first amino acid. Deprotection of the Lys(Dde)-group was carried out 2 times with 5% hydrazine hydrate in DMF (10 ml) for 3 min at 90° C. Raw products were washed on resin with DCM and dried prior to cleavage. Cleavage from resin and deprotection was performed with a mixture of 95% TFA/water (10 ml) and triisopropylsilane (250 μl) for 45 min at 40° C. Crude peptides were precipitated with cold tertbutyl-methyl ether, dissolved in 50% acetonitrile/water and purified by preparative HPLC.

### Purification Method 1 (L20)

Crude peptides were dissolved in DMF/acetonitrile/water and purified by reversed phase chromatography using an Agilent preparative HPLC-MS System with preparative pumps G7161B, G7111B and G7110B, a diode array detector G7115A, a mass-spectrometer G6135B and a fraction collector G7158B. A Waters Luna Prep C8 (3) column (100 Å, 10 μm, 300 g, self-filled steel column) served as stationary phase. The mobile phase was run with a gradient of buffer A (ACN) and buffer (H₂O+0.1% TFA) as described in the table below at a flow rate of 150 ml/min at 40° C. The relevant fractions were pooled and lyophilized. The final product was characterized by analytical HPLC-MS (U046_001 and U046_006, see below).

Purification method 1 standard gradient

| Time in minute | Flow [Pump A + B] | Pump A % | Pump B % |
|---|---|---|---|
| 0.0 | 120 ml/min | 20 | 80 |
| 6.0 | 120 ml/min | 20 | 80 |
| 45.0 | 120 ml/min | 40 | 60 |
| 52.0 | 120 ml/min | 40 | 60 |
| 55.0 | 120 ml/min | 45 | 55 |
| 62.0 | 120 ml/min | 45 | 55 |
| 63.0 | 120 ml/min | 100 | 0 |
| 70.0 | 120 ml/min | 100 | 0 |
| 70.1 | 120 ml/min | 100 | 0 |

### Analytical Method 1 (U046_001/U046_006/010_CA07)

Peptide purity and mass were estimated by analytical HPLC-MS on a Kinetex C8 column (4.6 mm×150 mm, 2.6 μm, Phenomenex) using a Agilent 1260 HPLC system equipped with Mass Detector G6135 and Waters Acquity with 3100 MS. Analysis was performed by gradient elution with buffer A (0.3% TFA in H₂O) and buffer B (0.24% TFA in ACN) at a temperature of 40° C. Details of the gradient and flow rates are summarized in the tables below. Retention times and masses were recorded.

Peptide purities (relative peak areas @ 214 nm) were in the range from 80% to 99%, preferably greater than 95%.

| | | Gradient/ | | | | |
|---|---|---|---|---|---|---|
| | | Solvent | % Sol | % Sol | | |
| | | Time | [Water 0.3% | [ACN 0.24% | Flow | Temp |
| Method name | U046_001 | [min] | TFA (v/v)] | TFA (v/v)] | ml/min | [° C.] |
| Device | Agilent 1260 with MS | 0.0 | 65.0 | 35.0 | 0.5 | 40.0 |
| | G6135 | | | | | |
| Column | Kinetex C8_4.6 × 150 | 15.0 | 45.0 | 55.0 | 0.5 | 40.0 |
| | mm_2.6 μm | | | | | |
| Column | Phenomenex | 16.0 | 10.0 | 90.0 | 1.0 | 40.0 |
| producer | | | | | | |
| | | 17.0 | 65.0 | 35.0 | 1.0 | 40.0 |
| | | 19.0 | 65.0 | 35.0 | 1.0 | 40.0 |

| | | Gradient/ | | | | |
|---|---|---|---|---|---|---|
| | | Solvent | % Sol | % Sol | | |
| Method | | Time | [Water 0.3% | [ACN 0.24% | Flow | Temp |
| Name | U046_006 | [min] | TFA (v/v)] | TFA (v/v)] | ml/min | [° C.] |
| Device | Agilent 1260 with MS | 0.0 | 85.0 | 15.0 | 0.5 | 40.0 |
| | G6135 | | | | | |
| Column | Kinetex C8_4.6 × 150 | 50.0 | 35.0 | 65.0 | 0.5 | 40.0 |
| | mm_2.6 μm | | | | | |
| Column | Phenomenex | 52.0 | 10.0 | 90.0 | 1.0 | 40.0 |
| producer | | | | | | |
| | | 53.0 | 85.0 | 15.0 | 1.0 | 40.0 |
| | | 55.0 | 85.0 | 15.0 | 1.0 | 40.0 |
| | | Gradient/ | | | | |
| | | Solvent | % Sol | % Sol | | |
| Method | | Time | [Water 0.3% | [ACN 0.24% | Flow | Temp |
| Name | 010_CA07 | [min] | TFA (v/v)] | TFA (v/v)] | ml/min | [° C.] |
| Device | Waters Acquity with | 0.0 | 65.0 | 35.0 | 0.5 | 40.0 |
| | 3100 MS | | | | | |
| Column | Kinetex C8_4.6 × 150 | 15.0 | 45.0 | 55.0 | 0.5 | 40.0 |
| | mm_2.6 μm | | | | | |
| Column | Phenomenex | 16.0 | 10.0 | 90.0 | 1.0 | 40.0 |
| producer | | | | | | |
| | | 17.01 | 65.0 | 35.0 | 1.0 | 40.0 |
| | | 18.0 | 65.0 | 35.0 | 1.0 | 40.0 |

### LIST OF FURTHER ABBREVIATIONS

ACN: acetonitrile
Boc: tert-butyloxycarbonyl
C18DA(tBu): 18-(tert-butoxy)-18-oxooctadecanoic acid
C20DA(tBu): 20-(tert-butoxy)-20-oxoicosanoic acid
DCM: dichloromethane
DIC: diisopropylcarbodiimide
DIPEA: disopropylethylamine
Dde: (4,4-dimethyl-2,6-dioxocyclohex-1-ylidene)ethyl
DMF: N,N-dimethylformamid
DODT: 3,6-dioxa-1,8-octanedithiol
DPBS: Dulbecco's phosphate-buffered saline
Fmoc: 9H-fluoren-9-ylmethoxycarbonyl
Fmoc-AHX: 6-{[(9H-fluoren-9-ylmethoxy)carbonyl]amino}hexanoic acid
Fmoc-OEG-OH: 2-[2-[2-(9H-fluoren-9-ylmethoxycarbonylamino)ethoxy]ethoxy] acetic acid
Fmoc-OEG-OEG-OH: 2-[2-[2-[[2-[2-[2-(9H-fluoren-9-ylmethoxycarbonyl-amino)ethoxy]ethoxy]acetyl]amino]ethoxy]ethoxy]acetic acid
HTRF: homogeneous time resolved fluorescence
IBMX: 3-isobutyl-1-methylxanthine
MRT: mean residence time
Mtt: 4-Methyl-trityl
NMP: 1-methyl-pyrrolidine-2-one
Oxyma: 2-cyano-2-(hydroxyimino) acetic acid ethyl ester
Pbf: 2,2,4,6,7-pentamethyldihydrobenzofuran-5-sulfonyl
Rt: retention time
RT: room temperature
SPPS: solid-phase peptide synthesis
tBu: tert-butyl
Trt: trityl
TES: triethylsilane
TFA: trifluoroacetic acid

The following compounds were synthesised. All compounds were obtained as TFA salts:

### Reference Compounds

Ref 1 (i.e. Example 41 of EP3467106)

(SEQ ID NOs 177 and 178)
NMeY-Aib-E-G-T-aMeF-T-S-D-K(1,3)-S-I-Aib-L-E-K-Q-R-Q-Iva-E-F-V-R-H-L-L-N-K-Aib-T-
R-Q-R-Y-NH2.C16A-G-G-G-G(1,2)

MW (calculated): 4720.5 Da
Synthesis and purification methods: S01; L20
   Ref 2 (i.e. Example 42 of EP3467106)

(SEQ ID NOS 179 and 180)
NMeY-Aib-E-G-T-aMeF-T-S-D-K(1,3)-S-I-Aib-L-E-K-Q-R-Q-Iva-E-F-V-R-H-L-L-N-K-Aib-T-R-Q-
R-Y-NH2.C18DA-G-G-G-G(1,2)

MW (calculated): 4778.6 Da
Synthesis and purification methods: S01; L20
   Ref 3 (i.e. Example 40 of EP3467106)

(SEQ ID NOS 188 and 189)
Y-Aib-E-G-T-aMeF-T-S-D-K(1,3)-S-I-Aib-L-E-K-Q-R-Q-Iva-E-F-V-R-H-L-L-N-K-Aib-T-R-Q-R-
Y-NH2.C18DA-G-G-G-G(1,2)

MW (calculated): 4764.5 Da
Synthesis and purification methods: S01; L20
LCMS: U046_006; Rt: 30.3 min; m/3: m/4: 1191.4

In Reference Compounds the term “Iva” means isovaline.

Compounds of the invention

Compound 1
(SEQ ID NO 1)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-AHX-AHX(1,2)

MW (calculated): 5250.0 Da
Synthesis and purification methods: S01; L20
LCMS: U046_001; Rt: 12.7 min; m/3: m/4: 1313.4

Compound 2
(SEQ ID NO 2)
Y-Aib-E-G-T-F-T-S-D-Y-S-I-Aib-L-E-K-Q-A-Q-Aib-A-F-V-R-H-L-I-N-K(1,3)-Aib-T-R-NMeQ-R-Y-
NH2.C18DA-gGlu-gGlu-gGlu-gGlu-gGlu-gGlu(1,2)

MW (calculated): 5188.8 Da
Synthesis and purification methods: S01; L20
LCMS: U046_001; Rt: 10.6 min; m/3: m/4: 1298.0

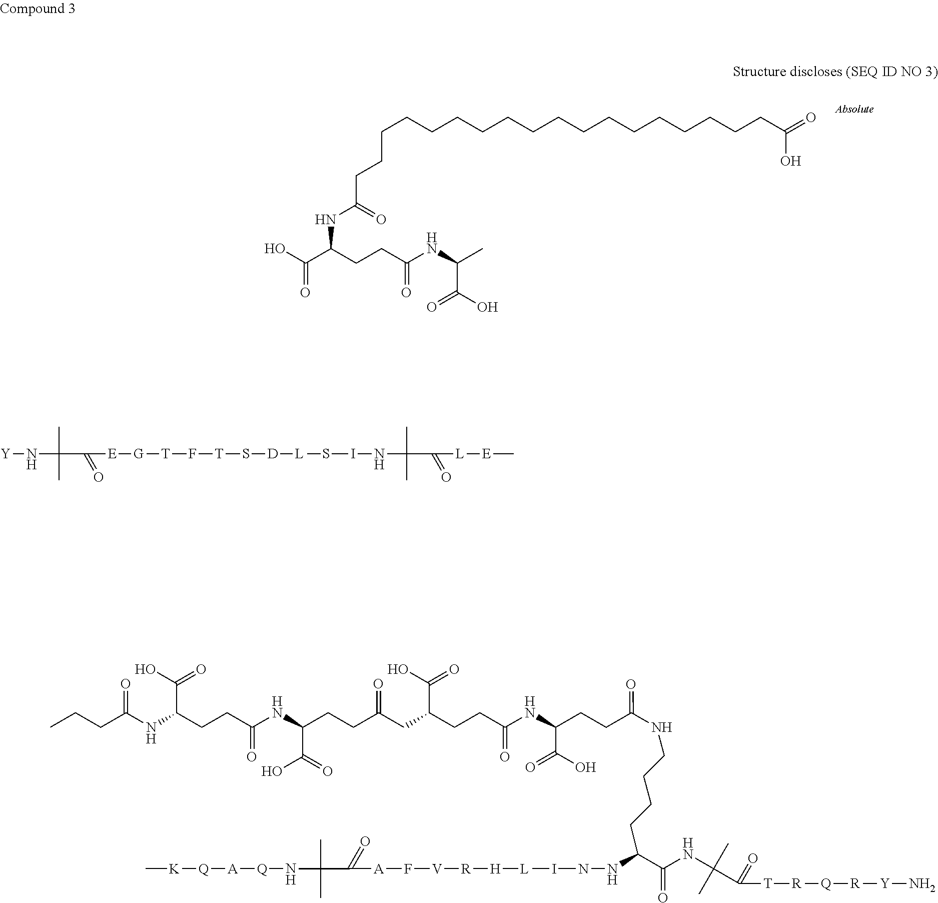

(SEQ ID NO 3)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu(1,2)

MW (calculated): 5202.9 Da
Synthesis and purification methods: S01; L20
LCMS: U046_001; Rt: 12 min; m/3: m/4: 1301.1

Compound 4
(SEQ ID NO 4)
Y-Aib-E-G-T-F-T-S-D-Y-S-I-Aib-L-E-K-A-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5274.0 Da
Synthesis and purification methods: S01; L20
LCMS: U046_001; Rt: 11.6 min; m/3: m/4: 1319.3

Compound 5
(SEQ ID NO 5)
Y-Aib-E-G-T-F-T-S-D-Y-S-I-Aib-L-E-Aib-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5259.9 Da
Synthesis and purification methods: S01; L20
LCMS: U046_006; Rt: 34.8 min; m/3: m/4: 1315.7

Compound 6
(SEQ ID NO 6)
Y-Aib-E-G-T-F-T-S-D-Y-S-I-Aib-L-E-N-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5288.9 Da
Synthesis and purification methods: S01; L20
LCMS: U046_006; Rt: 31.5 min; m/3: m/4: 1323.0

Compound 7
(SEQ ID NO 7)
Y-Aib-E-G-T-F-T-S-D-Y-S-I-Aib-L-E-K-Q-A-Q-Aib-A-F-V-R-H-L-I-N-K(1,3)-L-T-R-Q-R-Y-
NH2.C18DA-gGlu-gGlu-gGlu-gGlu-gGlu-OEG(1,2)

MW (calculated): 5218.9 Da
Synthesis and purification methods: S01; L20
LCMS: U046_001; Rt: 11.9 min; m/3: m/4: 1305.0

Compound 8
(SEQ ID NO 8)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-
NH2.C18DA-gGlu-gGlu-gGlu-gGlu-gGlu-OEG(1,2)

MW (calculated): 5248.9 Da
Synthesis and purification methods: S01; L20
LCMS: 010_CA07; Rt: 9.1 min; m/3: m/4: 1313.4

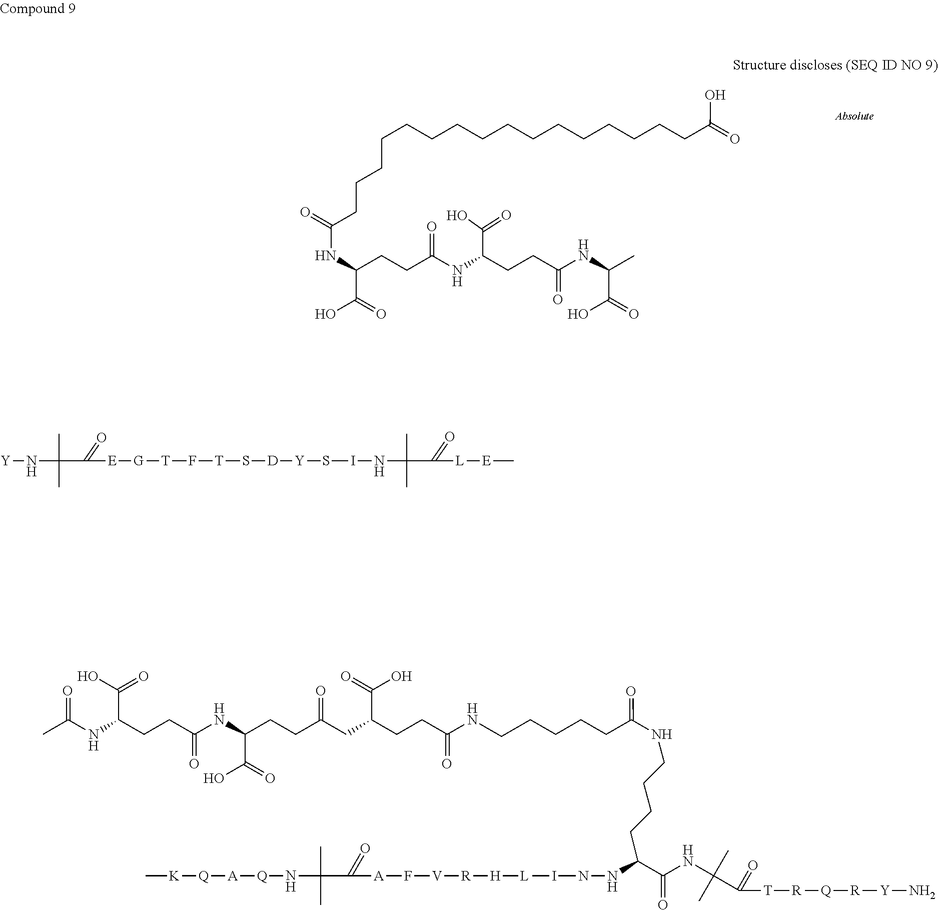

(SEQ ID NO 9)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Y-NH2.C18DA-gGlu-gGlu-gGlu-gGlu-gGlu-Glu-AHX(1,2)

MW (calculated): 5288.0 Da
Synthesis and purification methods: S01; L20
LCMS: U046_001; Rt: 10.3 min; m/3: m/4: 1322.7

Compound 10
(SEQ ID NO 10)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5304.0 Da
Synthesis and purification methods: S01; L20
LCMS: U046_001; Rt: 10.2 min; m/3: m/4: 1326.8

Compound 11
(SEQ ID NO 11)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys-eLys(1,2)

MW (calculated): 5431.2 Da
Synthesis and purification methods: S01; L20
LCMS: U046_001; Rt: 9.2 min; m/3: m/4: 1358.1

Compound 12
(SEQ ID NO 12)
Y-Aib-E-G-T-F-T-S-D-Y-S-I-Aib-L-E-K-Q-A-Q-Aib-A-F-V-R-H-L-I-N-K(1,3)-L-T-R-Q-R-Y-
NH2.C18DA-gGlu-gGlu-gGlu-gGlu-gGlu-gGlu(1,2)

MW (calculated): 5202.9 Da
Synthesis and purification methods: S01; L20
LCMS: U046_001; Rt: 11.1 min; m/3: m/4: 1301.4

Compound 13
(SEQ ID NO 13)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-AHX(1,2)

MW (calculated): 5158.9 Da
Synthesis and purification methods: S01; L20
LCMS: U046_001; Rt: 10.4 min; m/3: m/4: 1290.5

Compound 14
(SEQ ID NO 14)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-
NH2.C18DA-gGlu-gGlu-gGlu-gGlu-gGlu-AHX(1,2)

MW (calculated): 5216.9 Da
Synthesis and purification methods: S01; L20
LCMS: 010_CA07; Rt: 9.2 min; m/3: m/4: 1305.4

Compound 15
(SEQ ID NO 15)
Y-Aib-E-G-T-F-T-S-D-F-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5315.0 Da
Synthesis and purification methods: S01; L20
LCMS: U046_001; Rt: 11.9 min; m/3: m/4: 1329.6

Compound 16
(SEQ ID NO 16)
Y-Aib-E-G-T-F-T-S-D-Y-S-I-Aib-L-D-K-Q-A-Q-Aib-A-F-
V-R-H-L-I-N-K(1,3)-Aib-T-R-Q-dOR-Y-NH2.C20DA-gGlu-
gGlu-gGlu-gGlu-gGlu-gGlu-gGlu-eLys(1,2)

MW (calculated): 5432.1 Da
Synthesis and purification methods: S01; L20
LCMS: U046_006; Rt: 31.3 min; m/3: m/4: 1358.8

Compound 17
(SEQ ID NO 17)
Y-Aib-E-G-T-F-T-S-D-Y-S-I-Aib-L-E-K-Q-A-Q-Aib-A-F-
V-R-H-L-I-N-K(1,3)-Aib-T-R-NMeQ-R-Y-NH2.C18DA-
gGlu-gGlu-gGlu-gGlu-gGlu-gGlu-eLys(1,2)

MW (calculated): 5318.0 Da
Synthesis and purification methods: S01; L20
LCMS: U046_001; Rt: 9.9 min; m/3: m/4: 1330.0

Compound 18
(SEQ ID NO 18)
Y-Aib-E-G-T-F-T-S-D-Y-S-I-Aib-L-E-K-Q-A-Q-Aib-A-F-
V-R-H-L-I-N-K(1,3)-I-T-R-Q-R-Y-NH2.C18DA-gGlu-
gGlu-gGlu-gGlu-gGlu-gGlu-gGlu-eLys(1,2)

MW (calculated): 5460.2 Da
Synthesis and purification methods: S01; L20
LCMS: 010_CA07; Rt: 9.5 min; m/3: m/4: 1366.4

### Compound 19

Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2·C18DA-gGlu-gGlu-gGlu-gGlu-gGlu-OEG (1,2) (SEQ ID NO 19)
MW (calculated): 6057.8 Da
Synthesis and purification methods: S01; L20
LCMS: 010_CA07; Rt: 11.1 min; m/3: m/4: 1515.6

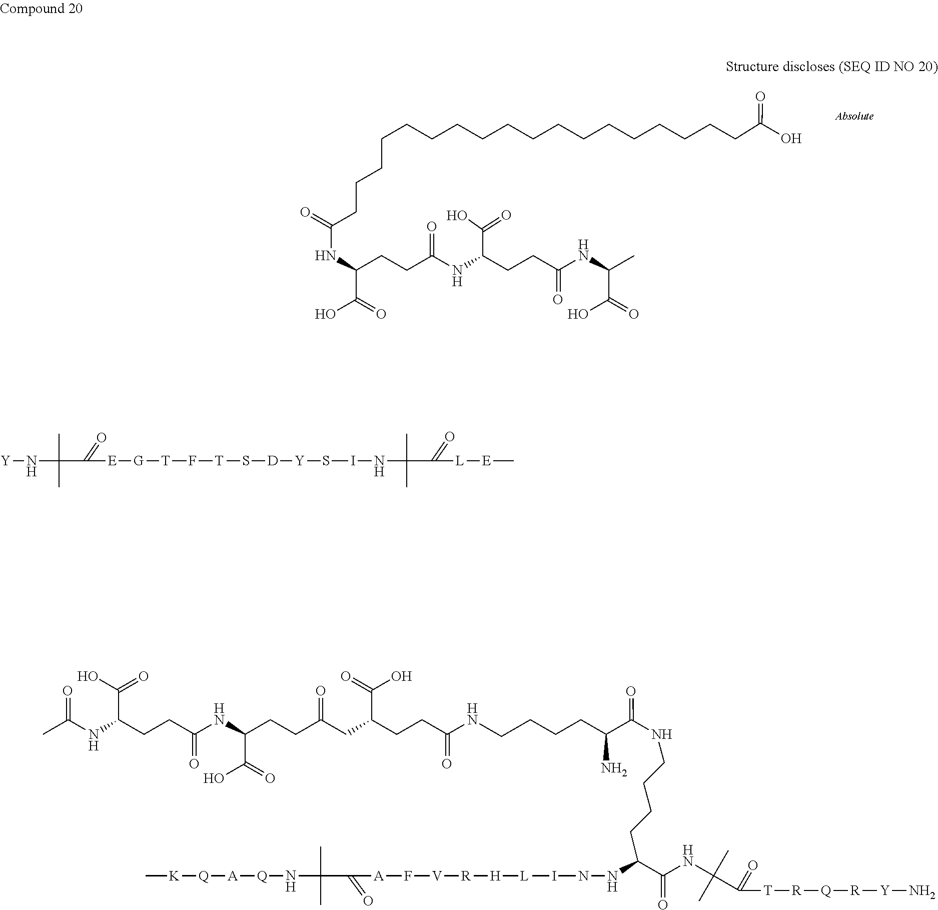

(SEQ ID NO 20)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5281.0 Da
Synthesis and purification methods: LP; L20
LCMS: U046_001; Rt: 12 min; m/3: m/4: 1321.0

Compound 21
(SEQ ID NO 21)
Y-Aib-E-G-T-F-T-S-D-L-S-I-Aib-L-E-K-Q-A-Q-Aib-A-F-
I-R-H-L-I-N-K(1,3)-Aib-T-R-Q-R-Y-NH2.C20DA-gGlu-
gGlu-gGlu-gGlu-gGlu-gGlu-eLys(1,2)

MW (calculated): 5295.0 Da
Synthesis and purification methods: S01; L20
LCMS: U046_001; Rt: 12.1 min; m/3: m/4: 1324.6

Compound 22
(SEQ ID NO 22)
Y-Aib-E-G-T-F-T-S-D-Y-S-I-aMeL-L-D-K-Q-A-Q-Aib-A-
F-I-R-H-L-I-N-K(1,3)-Aib-T-R-Q-R-Y-NH2.C20DA-
gGlu-gGlu-Glu-gGlu-gGlu-gGlu-eLys(1,2)

MW (calculated): 5373.1 Da
Synthesis and purification methods: S01; L20
LCMS: U046_006; Rt: 33.4 min; m/3: m/4: 1344.0

Compound 23
(SEQ ID NO 23)
Y-Aib-E-G-T-F-T-S-D-Y-S-I-Aib-L-E-Q-Q-A-Q-Aib-A-F-
V-R-H-L-I-N-K(1,3)-Aib-T-R-Q-R-Y-NH2.C18DA-gGlu-
gGlu-gGlu-gGlu-gGlu-gGlu-eLys(1,2)

MW (calculated): 5302.9 Da
Synthesis and purification methods: S01; L20
LCMS: U046_006; Rt: 31.6 min; m/3: m/4: 1326.4

Compound 24
(SEQ ID NO 24)
Y-Aib-E-G-T-F-T-S-D-Y-S-I-Aib-L-E-K-I-A-Q-Aib-A-F-
V-R-H-L-I-N-K(1,3)-Aib-T-R-Q-R-Y-NH2.C18DA-gGlu-
gGlu-gGlu-gGlu-gGlu-gGlu-eLys(1,2)

MW (calculated): 5288.0 Da
Synthesis and purification methods: S01; L20
LCMS: U046_006; Rt: 32.5 min; m/3: m/4: 1322.7

Compound 25
(SEQ ID NO 25)
Y-Aib-E-G-T-F-T-S-D-Y-S-I-Aib-L-E-E-E-A-Q-Aib-A-F-
V-R-H-L-I-N-K(1,3)-Aib-T-R-Q-R-Y-NH2.C18DA-gGlu-
gGlu-gGlu-gGlu(1,2)

MW (calculated): 4918.5 Da
Synthesis and purification methods: S01; L20
LCMS: U046_001; Rt: 12.4 min; m/3:1640.3 m/4:

Compound 26
(SEQ ID NO 26)
Y-Aib-E-G-T-F-T-S-D-Y-S-I-Aib-L-E-K-Q-A-Q-Aib-A-F-
V-R-H-L-I-N-K(1,3)-Aib-T-R-NMeQ-R-Y-NH2.C20DA-
gGlu-gGlu-gGlu-gGlu-gGlu-gGlu-eLys(1,2)

MW (calculated): 5345.1 Da
Synthesis and purification methods: S01; L20
LCMS: U046_001; Rt: 11.4 min; m/3: m/4: 1337.1

Compound 27
(SEQ ID NO 27)
Y-Aib-E-G-T-F-T-S-D-Y-S-I-Aib-L-E-K-Q-A-Q-Aib-A-F-
V-R-H-L-I-N-K(1,3)-L-T-R-Q-R-Y-NH2.C20DA-gGlu-
gGlu-gGlu-gGlu-gGlu-gGlu-eLys(1,2)

MW (calculated): 5359.1 Da
Synthesis and purification methods: S01; L20
LCMS: U046_001; Rt: 12 min; m/3: m/4: 1340.6

Compound 28
(SEQ ID NO 28)
Y-Aib-E-G-T-F-T-S-D-Y-S-I-Aib-L-E-K-E-A-Q-Aib-A-F-
V-R-H-L-I-N-K(1,3)-Aib-T-R-Q-R-Y-NH2.C20DA-gGlu-
gGlu-gGlu-gGlu-gGlu-Glu-eLys(1,2)

MW (calculated): 5332.0 Da
Synthesis and purification methods: S01; L20
LCMS: U046_001; Rt: 11.5 min; m/3: m/4: 1333.7

Compound 29
(SEQ ID NO 29)
Y-Aib-E-G-T-F-T-S-D-Y-S-I-A-L-E-K-Q-A-Q-Aib-A-F-V-
R-H-L-I-N-K(1,3)-Aib-T-R-Q-R-Y-NH2.C20DA-gGlu-
gGlu-gGlu-gGlu-gGlu-gGlu(1,2)

MW (calculated): 5188.8 Da
Synthesis and purification methods: S01; L20
LCMS: U046_001; Rt: 11.8 min; m/3: m/4: 1298.0

Compound 30
(SEQ ID NO 30)
Y-Aib-E-G-T-F-T-S-D-Y-S-I-Aib-L-E-K-Q-A-Q-Aib-E-F-
V-R-H-L-I-N-K(1,3)-Aib-T-R-Q-R-Y-NH2.C18DA-gGlu-
gGlu-gGlu-gGlu-gGlu-gGlu-gGlu-eLys(1,2)

MW (calculated): 5490.1 Da
Synthesis and purification methods: S01; L20
LCMS: U046_001; Rt: 11.3 min; m/3: m/4: 1373.3

Compound 31
(SEQ ID NO 31)
Y-Aib-E-G-T-F-T-S-D-Y-S-I-Aib-L-E-K-Q-A-Q-Aib-A-F-
V-R-H-L-I-N-K(1,3)-Aib-T-R-Q-R-Y-NH2.C18DA-TRX-
gGlu-gGlu-gGlu-gGlu-gGlu-gGlu(1,2)

MW (calculated): 5314.0 Da
Synthesis and purification methods: S01; L20
LCMS: U046_001; Rt: 11.6 min; m/3: m/4: 1329.2

Compound 32
(SEQ ID NO 32)
Y-Aib-E-G-T-F-T-S-D-Y-S-I-Aib-L-E-K-Q-A-Q-Aib-E-F-
V-R-H-L-I-N-K(1,3)-L-T-R-Q-R-Y-NH2.C18DA-gGlu-
gGlu-gGlu-gGlu-gGlu-gGlu-Glu-eLys(1,2)

MW (calculated): 5518.2 Da
Synthesis and purification methods: S01; L20
LCMS: U046_001; Rt: 10.2 min; m/3: m/4: 1380.2

Compound 33
(SEQ ID NO 33)
Y-Aib-E-G-T-F-T-S-D-Y-S-I-Aib-L-D-K-I-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5274.0 Da
Synthesis and purification methods: S01; L20
LCMS: U046_006; Rt: 32.5 min; m/3: m/4: 1319.3

Compound 34
(SEQ ID NO 34)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-eLys-gGlu-gGlu(1,2)

MW (calculated): 5303.0 Da
Synthesis and purification methods: S01; L20
LCMS: U046_001; Rt: 9.6 min; m/3: m/4: 1326.5

Compound 35
(SEQ ID NO 35)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-gGlu-eLys(1,2)

MW (calculated): 5460.2 Da
Synthesis and purification methods: S01; L20
LCMS: U046_001; Rt: 11.2 min; m/3: m/4: 1365.8

Compound 36
(SEQ ID NO 36)
Y-Aib-E-G-T-F-T-S-D-Y-S-I-Aib-L-E-K-Q-E-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5361.0 Da
Synthesis and purification methods: S01; L20
LCMS: U046_001; Rt: 9.7 min; m/3: m/4: 1341.0

Compound 37
(SEQ ID NO 37)
Y-Aib-E-G-T-F-T-S-D-Y-S-I-Aib-L-E-K-Q-A-Q-Aib-A-F-L-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5317.0 Da
Synthesis and purification methods: S01; L20
LCMS: U046_001; Rt: 9.6 min; m/3: m/4: 1330.1

Compound 38
(SEQ ID NO 38)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-gGlu(1,2)

MW (calculated): 5303.9 Da
Synthesis and purification methods: S01; L20
LCMS: U046_001; Rt: 10.4 min; m/3: m/4: 1326.8

Compound 39
(SEQ ID NO 39)
Y-Aib-E-G-T-F-T-S-D-Y-S-I-Aib-L-E-K-Q-A-Q-Aib-E-F-V-R-H-L-I-N-K(1,3)-L-T-R-Q-R-Y-
NH2.C20DA-gGlu-gGlu-gGlu-gGlu-gGlu-gGlu-eLys(1,2)

MW (calculated): 5417.1 Da
Synthesis and purification methods: S01; L20
LCMS: U046_001; Rt: 12 min; m/3: m/4: 1355.0

(SEQ ID NO 40)
Compound 40
Y-Aib-E-G-T-F-T-S-D-Y-S-I-Aib-L-D-E-Q-A-Q-Aib-A-F-V-R-H-L-I-N-K(1,3)-Aib-T-R-Q-R-Y-
NH2.C18DA-gGlu-gGlu-gGlu-gGlu-Glu-gGlu-eLys(1,2)

MW (calculated): 5289.9 Da
Synthesis and purification methods: S01; L20
LCMS: U046_006; Rt: 32.4 min; m/3: m/4: 1323.2

Compound 41
(SEQ ID NO 41)
Y-Aib-E-G-T-F-T-S-D-Y-S-I-Aib-L-E-K-Q-A-Q-Aib-A-F-V-R-H-L-I-N-K(1,3)-I-T-R-Q-R-Y-
NH2.C18DA-gGlu-gGlu-gGlu-gGlu-gGlu(1,2)

MW (calculated): 5073.7 Da
Synthesis and purification methods: S01; L20
LCMS: U046_001; Rt: 11.3 min; m/3: m/4: 1269.0

Compound 42
(SEQ ID NO 42)
Y-Aib-E-G-T-F-T-S-D-Y-S-I-Aib-L-E-Q-Q-A-Q-Aib-A-F-V-R-H-L-I-N-K(1,3)-Aib-T-R-Q-R-Y-
NH2.C18DA-gGlu-gGlu-gGlu-gGlu-gGlu(1,2)

MW (calculated): 5045.6 Da
Synthesis and purification methods: S01; L20
LCMS: 010_CA07; Rt: 9.9 min; m/3: m/4: 1262.6

Compound 43
(SEQ ID NO 43)
Y-Aib-E-G-T-F-T-S-D-Y-S-I-Aib-L-E-K-Q-A-Q-Aib-A-F-V-R-H-L-I-N-K(1,3)-Aib-T-R-Q-NMeR-Y-
NH2.C18DA-gGlu-gGlu-gGlu-gGlu-gGlu-gGlu-eLys(1,2)

MW (calculated): 5317.0 Da
Synthesis and purification methods: S01; L20
LCMS: U046_001; Rt: 9.9 min; m/3: m/4: 1330.1

Compound 44
(SEQ ID NO 44)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-AHX-AHX(1,2)

MW (calculated): 5272.0 Da
Synthesis and purification methods: S01; L20
LCMS: U046_006; Rt: 30.8 min; m/3: m/4: 1318.7

Compound 45
(SEQ ID NO 45)
Y-Aib-E-G-T-F-T-S-D-Y-S-I-Aib-L-E-K-Q-A-Q-Aib-E-F-V-R-H-L-I-N-K(1,3)-L-T-R-Q-R-Y-
NH2.C18DA-gGlu-gGlu-gGlu-Glu-gGlu-AHX(1,2)

MW (calculated): 5244.9 Da
Synthesis and purification methods: S01; L20
LCMS: U046_001; Rt: 11.4 min; m/3: m/4: 1312.0

Compound 46
(SEQ ID NO 46)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-gDab(1,2)

MW (calculated): 5303.0 Da
Synthesis and purification methods: S01; L20
LCMS: U046_001; Rt: 11.3 min; m/3: m/4: 1326.5

Compound 47
(SEQ ID NO 47)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-OEG(1,2)

MW (calculated): 5320.0 Da
Synthesis and purification methods: S01; L20
LCMS: U046_001; Rt: 10.1 min; m/3: m/4: 1330.7

Compound 48
(SEQ ID NO 48)
Y-Aib-E-G-T-F-T-S-D-Y-S-I-Aib-L-E-E-Q-A-Q-Aib-A-F-V-R-H-L-I-N-K(1,3)-Aib-T-R-Q-R-Y-
NH2.C18DA-gGlu-gGlu-gGlu-gGlu-gGlu-eLys(1,2)

MW (calculated): 5174.8 Da
Synthesis and purification methods: S01; L20
LCMS: U046_006; Rt: 32.4 min; m/3: m/4: 1294.4

Compound 49
(SEQ ID NO 49)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu(1,2)

MW (calculated): 5244.9 Da
Synthesis and purification methods: S01; L20
LCMS: 010_CA07; Rt: 11.7 min; m/3: m/4: 1312.4

Compound 50
(SEQ ID NO 50)
Y-Aib-E-G-T-F-T-S-D-Y-S-I-Aib-L-E-K-Q-A-Q-Aib-A-F-V-R-Q-L-I-N-K(1,3)-Aib-T-R-Q-R-Y-
NH2.C18DA-gGlu-gGlu-gGlu-gGlu-gGlu(1,2)

MW (calculated): 5036.7 Da
Synthesis and purification methods: S01; L20
LCMS: U046_001; Rt: 10.7 min; m/3: m/4: 1260.0

Compound 51
(SEQ ID NO 51)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu(1,2)

MW (calculated): 5152.8 Da
Synthesis and purification methods: S01; L20
LCMS: U046_001; Rt: 12.7 min; m/3:1718.4 m/4:

Compound 52
(SEQ ID NO 52)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-eLys-gGlu-gGlu(1,2)

MW (calculated): 5331.0 Da
Synthesis and purification methods: S01; L20
LCMS: U046_001; Rt: 11.4 min; m/3: m/4: 1333.5

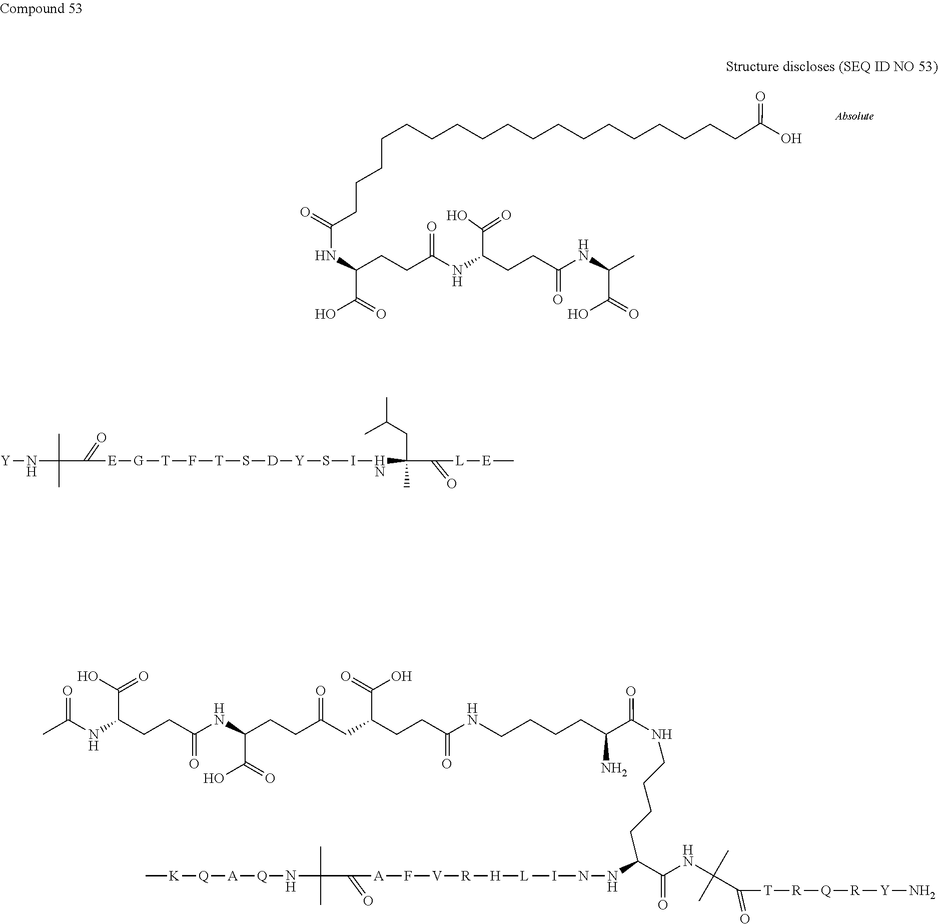

(SEQ ID NO 53)
Y-Aib-E-G-T-F-T-S-D-Y-S-I-aMeL-L-E-K-Q-A-Q-Aib-A-F-I-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5387.1 Da
Synthesis and purification methods: S01; L20
LCMS: U046_006; Rt: 32.1 min; m/3: m/4: 1347.0

Compound 54
(SEQ ID NO 54)
Y-Aib-E-G-E-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5331.0 Da
Synthesis and purification methods: S01; L20
LCMS: U046_001; Rt: 9.3 min; m/3: m/4: 1333.5

Compound 55
(SEQ ID NO 55)
Y-Aib-E-G-T-F-T-S-D-Y-S-I-Aib-L-D-A-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5231.9 Da
Synthesis and purification methods: S01; L20
LCMS: U046_006; Rt: 34.1 min; m/3: m/4: 1308.7

Compound 56
(SEQ ID NO 56)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-
NH2.C18DA-gGlu-gGlu-gGlu-gGlu-gGlu-Glu(1,2)

MW (calculated): 5232.8 Da
Synthesis and purification methods: S01; L20
LCMS: 010_CA07; Rt: 8.9 min; m/3: m/4: 1309.4

Compound 57
(SEQ ID NO 57)
Y-Aib-E-G-T-F-T-S-D-Y-S-I-L-L-E-K-Q-A-Q-Aib-A-F-V-R-H-L-I-N-K(1,3)-Aib-T-R-Q-R-Y-
NH2.C20DA-gGlu-Glu-gGlu-gGlu-gGlu-gGlu(1,2)

MW (calculated): 5230.9 Da
Synthesis and purification methods: S01; L20
LCMS: 010_CA07; Rt: 11.6 min; m/3: m/4: 1308.9

Compound 58
(SEQ ID NO 58)
Y-Aib-E-G-T-F-T-S-D-Y-S-I-Aib-L-E-K-Q-A-Q-Aib-A-F-V-R-H-L-Y-N-K(1,3)-Aib-T-R-Q-R-Y-
NH2.C18DA-gGlu-gGlu-gGlu-gGlu-gGlu-gGlu-eLys(1,2)

MW (calculated): 5353.0 Da
Synthesis and purification methods: S01; L20
LCMS: U046_001; Rt: 8.9 min; m/3: m/4: 1339.0

Compound 59
(SEQ ID NO 59)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-eLys(1,2)

MW (calculated): 5201.9 Da
Synthesis and purification methods: S01; L20
LCMS: U046_001; Rt: 11.4 min; m/3: m/4: 1301.2

Compound 60
(SEQ ID NO 60)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-eLys-gGlu(1,2)

MW (calculated): 5281.0 Da
Synthesis and purification methods: S01; L20
LCMS: U046_001; Rt: 12.1 min; m/3: m/4: 1321.1

Compound 61
(SEQ ID NO 61)
Y-Aib-E-G-T-F-T-S-D-Y-S-I-Aib-L-E-K-Q-A-Q-A-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5289.0 Da
Synthesis and purification methods: S01; L20
LCMS: U046_001; Rt: 9.6 min; m/3: m/4: 1323.0

Compound 62
(SEQ ID NO 62)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-Glu(1,2)

MW (calculated): 5045.7 Da
Synthesis and purification methods: S01; L20
LCMS: U046_001; Rt: 10.5 min; m/3: m/4: 1262.0

Compound 63
(SEQ ID NO 63)
Y-Aib-E-G-T-F-T-S-D-Y-S-I-Aib-L-E-LysAc-Q-A-Q-Aib-A-F-V-R-H-L-I-N-K(1,3)-Aib-T-R-Q-R-Y-
NH2.C18DA-gGlu-gGlu-gGlu-Glu(1,2)

MW (calculated): 4958.6 Da
Synthesis and purification methods: S01; L20
LCMS: U046_001; Rt: 12.7 min; m/3: m/4: 1240.5

Compound 64
(SEQ ID NO 64)
Y-Aib-E-G-T-F-T-S-D-Y-S-I-Aib-L-E-E-E-A-Q-Aib
-E-F-V-R-H-L-I-N-K(1,3)-Aib-T-R-Q-R-Y-NH2.C18
DA-gGlu-gGlu-gGlu-gGlu(1,2)

MW (calculated): 4976.5 Da
Synthesis and purification methods: S01; L20
LCMS: U046_001; Rt: 12.6 min; m/3: m/4: 1244.9

Compound 65
(SEQ ID NO 65)
Y-Aib-E-G-T-F-T-S-D-Y-S-I-Aib-L-E-L-Q-A-Q-Aib
-A-F-V-R-H-L-I-N-K(1,3)-Aib-T-R-Q-R-Y-NH2.C18
DA-gGlu-gGlu-gGlu-gGlu-gGlu-gGlu-eLys(1,2)

MW (calculated): 5288.0 Da
Synthesis and purification methods: S01; L20
LCMS: U046_001; Rt: 13.9 min; m/3: m/4: 1322.8

Compound 66
(SEQ ID NO 66)
Y-Aib-E-G-T-F-T-S-D-Y-S-I-Aib-L-E-K-Q-A-Q-Aib
-A-F-V-R-H-L-I-N-K(1,3)-I-T-R-Q-R-Y-NH2.C18DA
-gGlu-gGlu-gGlu-gGlu-gGlu-gGlu-eLys(1,2)

MW (calculated): 5331.0 Da
Synthesis and purification methods: S01; L20
LCMS: 010_CA07; Rt: 9.7 min; m/3: m/4: 1334.2

Compound 67
(SEQ ID NO 67)
Y-Aib-E-G-T-F-T-S-D-Y-S-I-aMeL-L-D-K-Q-A-Q-Ai
b-A-F-V-R-H-L-I-N-K(1,3)-Aib-T-R-Q-R-Y-NH2.C1
8DA-gGlu-gGlu-gGlu-gGlu-gGlu-gGlu-eLys(1,2)

MW (calculated): 5331.0 Da
Synthesis and purification methods: S01; L20
LCMS: U046_006; Rt: 31 min; m/3: m/4: 1333.5

Compound 68
(SEQ ID NO 68)
Y-Aib-E-G-T-F-T-S-D-Y-S-I-Aib-L-E-K-Q-A-Q-Aib
-A-F-V-R-H-L-I-A-K(1,3)-Aib-T-R-Q-R-Y-NH2.C18
DA-gGlu-gGlu-gGlu-gGlu-gGlu-gGlu-eLys(1,2)

MW (calculated): 5260.0 Da
Synthesis and purification methods: S01; L20
LCMS: U046_001; Rt: 9.5 min; m/3: m/4: 1315.8

Compound 69
(SEQ ID NO 69)
Y-Aib-E-G-T-F-T-S-D-Y-S-I-Aib-L-E-K-Q-A-Q-Aib
-A-F-V-R-H-L-I-N-K(1,3)-L-T-R-Q-R-Y-NH2.C18DA
-gGlu-gGlu-gGlu-gGlu-gGlu-AHX(1,2)

MW (calculated): 5186.9 Da
Synthesis and purification methods: S01; L20
LCMS: U046_001; Rt: 11.4 min; m/3: m/4: 1297.5

Compound 70
(SEQ ID NO 70)
Y-Aib-E-G-T-F-T-S-D-Y-S-I-Aib-L-D-Q-Q-A-Q-Aib
-A-F-V-R-H-L-I-N-K(1,3)-Aib-T-R-Q-R-Y-NH2.C18
DA-gGlu-gGlu-gGlu-gGlu-gGlu-eLys(1,2)

MW (calculated): 5159.8 Da
Synthesis and purification methods: S01; L20
LCMS: U046_006; Rt: 31.9 min; m/3: m/4: 1290.7

Compound 71
(SEQ ID NO 71)
Y-Aib-E-G-T-F-T-S-D-Y-S-I-Aib-L-E-E-Q-A-Q-Aib
-A-F-V-R-H-L-I-N-K(1,3)-Aib-T-R-Q-R-Y-NH2.C18
DA-gGlu-Glu-gGlu-gGlu-Glu-gGlu-eLys(1,2)

MW (calculated): 5303.9 Da
Synthesis and purification methods: S01; L20
LCMS: U046_001; Rt: 11.3 min; m/3: m/4: 1326.7

Compound 72
(SEQ ID NO 72)
Y-Aib-E-G-T-F-T-S-D-Y-S-I-Aib-L-E-K-Q-A-Q-Aib
-A-F-V-R-H-L-I-N-K(1,3)-Aib-T-R-Q-ArgMe-Y-NH2.
C18DA-gGlu-gGlu-gGlu-gGlu-gGlu-gGlu-eLys
(1,2)

MW (calculated): 5317.0 Da
Synthesis and purification methods: S01; L20
LCMS: U046_001; Rt: 9.7 min; m/3: m/4: 1330.1

Compound 73
(SEQ ID NO 73)
Y-Aib-E-G-T-F-T-S-D-Y-S-I-Aib-L-E-K-Q-A-Q-Aib
-E-F-V-R-H-L-I-N-K(1,3)-Aib-T-R-Q-R-Y-NH2.C18
DA-gGlu-gGlu-gGlu-gGlu-OEG-OEG(1,2)

MW (calculated): 5264.9 Da
Synthesis and purification methods: S01; L20
LCMS: U046_001; Rt: 10.2 min; m/3: m/4: 1317.0

Compound 74
(SEQ ID NO 74)
Y-Aib-E-G-T-F-T-S-D-Y-S-I-Aib-L-D-K-Q-A-Q-Aib
-A-F-V-R-H-L-I-N-K(1,3)-Aib-T-R-Q-R-Y-NH2.C20
DA-gGlu-gGlu-gGlu-gGlu-Glu-gGlu-eLys(1,2)

MW (calculated): 5317.0 Da
Synthesis and purification methods: LP; L20
LCMS: U046_006; Rt: 32.5 min; m/3: m/4: 1330.0

Compound 75
(SEQ ID NO 75)
Y-Aib-E-G-T-F-T-S-D-Y-S-I-Aib-L-E-E-Q-A-Q-Aib
-A-F-V-R-H-L-I-N-K(1,3)-Aib-T-R-Q-R-Y-NH2.C18
DA-gGlu-gGlu-gGlu-gGlu-gGlu(1,2)

MW (calculated): 5046.6 Da
Synthesis and purification methods: S01; L20
LCMS: 010_CA07; Rt: 10.6 min; m/3: m/4: 1262.8

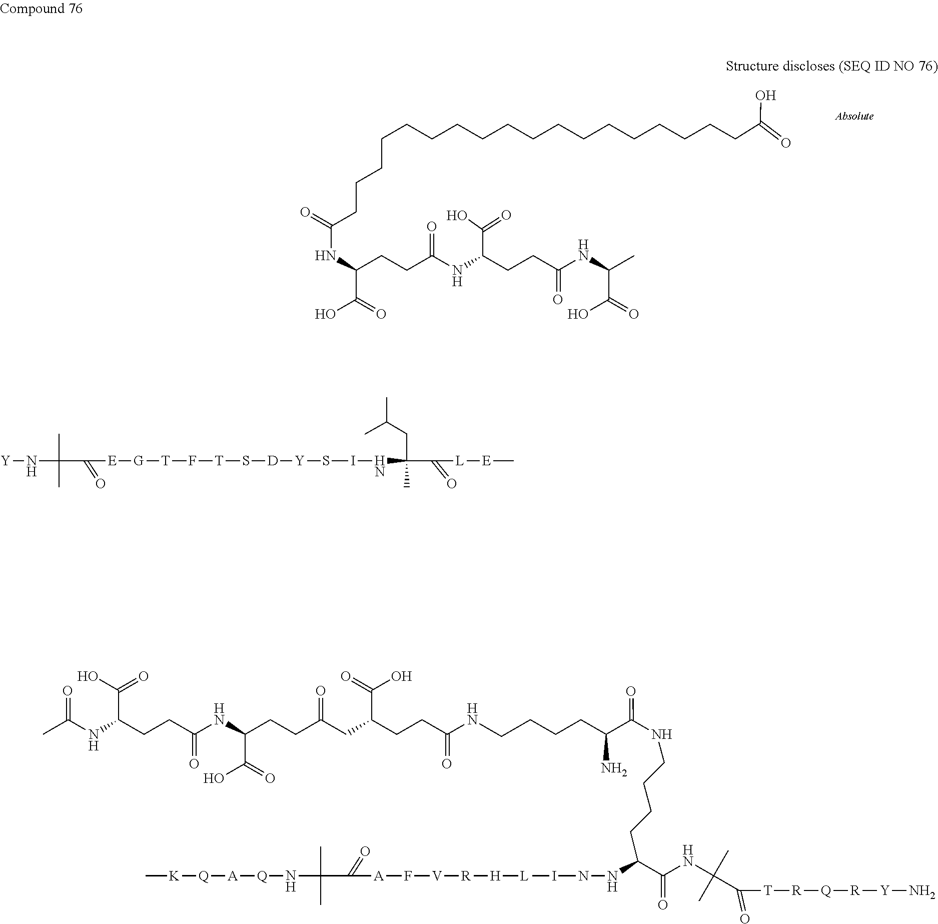

(SEQ ID NO 76)
Y-Aib-E-G-T-F-T-S-D-Y-S-I-aMeL-L-E-K-Q-A-Q-Aib-A-F-I-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5359.1 Da
Synthesis and purification methods: LP; L20
LCMS: U046_006; Rt: 31.1 min; m/3: m/4: 1340.5

Compound 77
(SEQ ID NO 77)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C18
DA-gGlu-gGlu-gGlu-gGlu(1,2)

MW (calculated): 4916.6 Da
Synthesis and purification methods: S01; L20
LCMS: U046_001; Rt: 10.7 min; m/3:1639.5 m/4:

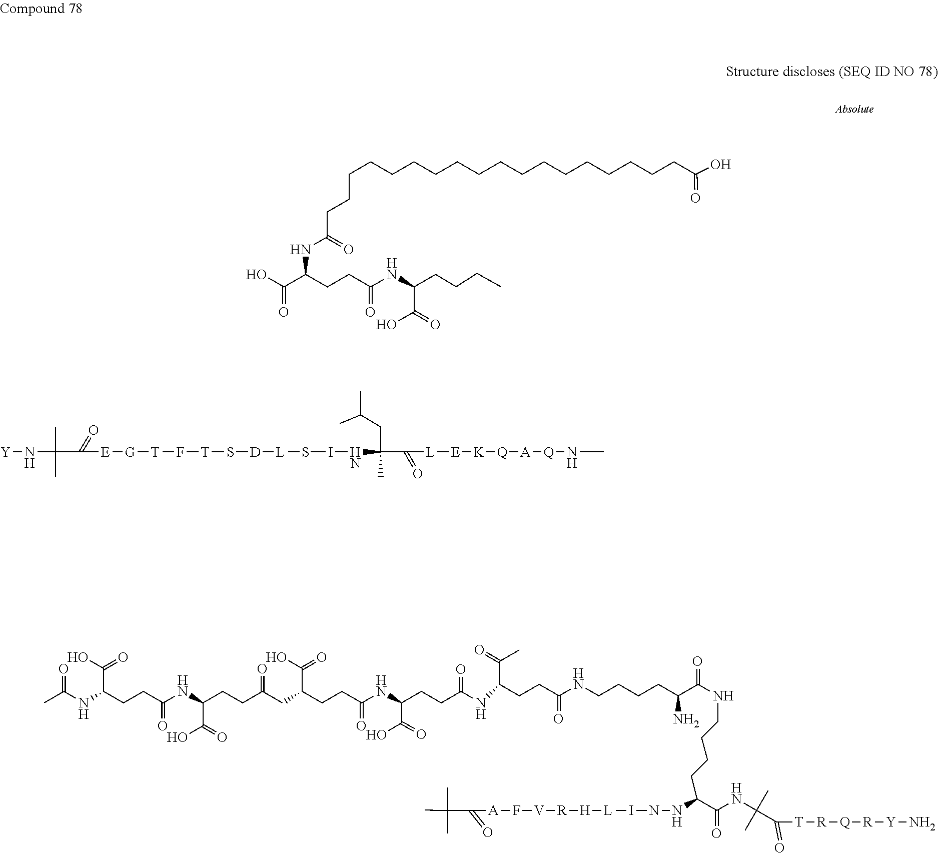

(SEQ ID NO 78)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-gGlu-eLys(1,2)

MW (calculated): 5502.2 Da
Synthesis and purification methods: S01; L20
LCMS: U046_001; Rt: 11.5 min; m/3: m/4: 1376.4

Compound 79
(SEQ ID NO 79)
Y-Aib-E-G-T-F-T-S-D-Y-S-I-Aib-L-E-K-Q-A-Q-Aib
-E-F-V-R-H-L-I-N-K(1,3)-Aib-T-R-Q-R-Y-NH2.C18
DA-gGlu-gGlu-gGlu-gGlu(1,2)

MW (calculated): 4974.6 Da
Synthesis and purification methods: S01; L20
LCMS: U046_001; Rt: 10.7 min; m/3: m/4: 1244.4

Compound 80
(SEQ ID NO 80)
Y-Aib-E-G-T-F-T-S-D-Y-S-I-Aib-L-E-K-Q-A-Q-Aib
-A-F-V-R-H-L-L-N-K(1,3)-Aib-T-R-Q-R-Y-NH2.C20
DA-gGlu-gGlu-gGlu-gGlu-gGlu-gGlu-eLys(1,2)

MW (calculated): 5331.0 Da
Synthesis and purification methods: S01; L20
LCMS: U046_001; Rt: 11.2 min; m/3: m/4: 1333.6

Compound 81
(SEQ ID NO 81)
Y-Aib-E-G-T-F-T-S-D-Y-S-I-Aib-L-D-K-Q-A-Q-Aib-A-F-I-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5331.0 Da
Synthesis and purification methods: S01; L20
LCMS: U046_006; Rt: 32.9 min; m/3: m/4: 1333.5

Compound 82
(SEQ ID NO 82)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-
NH2.C20DA-gGlu-gGlu-gGlu-gGlu-gGlu-gGlu-eLys-eLys(1,2)

MW (calculated): 5460.2 Da
Synthesis and purification methods: S01; L20
LCMS: U046_001; Rt: 11 min; m/3: m/4: 1365.8

Compound 83
(SEQ ID NO 83)
Y-Aib-E-G-T-F-T-S-D-Y-S-I-Aib-L-E-K-Q-A-Q-Aib-E-F-V-R-H-L-I-N-K(1,3)-L-T-R-Q-R-Y-
NH2.C18DA-gGlu-gGlu-gGlu-gGlu-gGlu-gGlu-eLys(1,2)

MW (calculated): 5389.1 Da
Synthesis and purification methods: S01; L20
LCMS: U046_001; Rt: 10.4 min; m/3: m/4: 1348.1

Compound 84
(SEQ ID NO 84)
Y-Aib-E-G-T-F-T-S-D-Y-S-I-Aib-L-E-K-Q-A-Y-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5338.0 Da
Synthesis and purification methods: S01; L20
LCMS: U046_001; Rt: 10.4 min; m/3: m/4: 1335.3

Compound 85
(SEQ ID NO 85)
Y-Aib-E-G-A-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5273.0 Da
Synthesis and purification methods: S01; L20
LCMS: U046_001; Rt: 9.5 min; m/3: m/4: 1319.0

Compound 86
(SEQ ID NO 86)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Phe4F-NH2.C18DA-gGlu-gGlu-gGlu-gGlu-Glu-gGlu-eLys(1,2)

MW (calculated): 5305.0 Da
Synthesis and purification methods: S01; L20
LCMS: U046_001; Rt: 10.3 min; m/3: m/4: 1327.0

Compound 87
(SEQ ID NO 87)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-dtOrn(1,2)

MW (calculated): 5289.0 Da
Synthesis and purification methods: S01; L20
LCMS: U046_001; Rt: 9.5 min; m/3: m/4: 1323.0

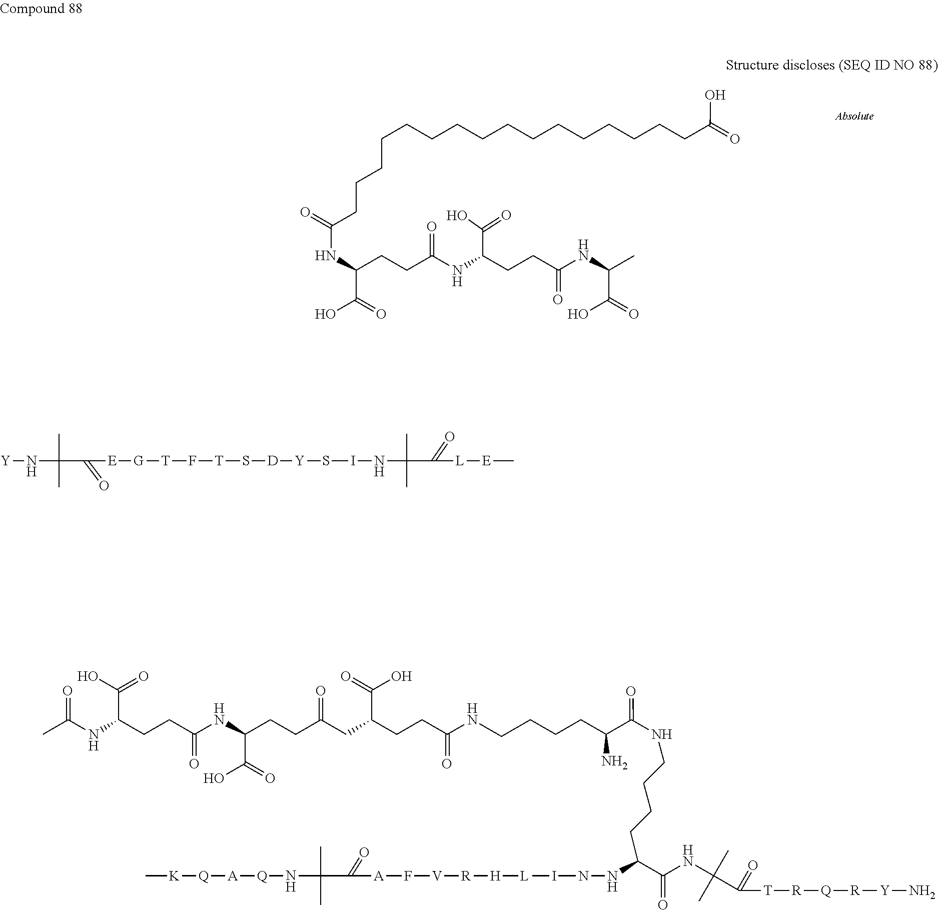

(SEQ ID NO 88)
Y-Aib-E-G-T-F-T-S-D-Y-S-I-Aib-L-E-K-Q-A-Q-Aib-A-F-I-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5317.0 Da
Synthesis and purification methods: S01; L20
LCMS: U046_006; Rt: 38.9 min; m/3: m/4: 1330.0

Compound 89
(SEQ ID NO 89)
Y-Aib-E-G-T-F-T-S-D-Y-S-I-aMeL-L-E-E-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5374.1 Da
Synthesis and purification methods: LP; L20
LCMS: U046_006; Rt: 34.3 min; m/3: m/4: 1344.2

Compound 90
(SEQ ID NO 90)
Y-Aib-E-G-T-F-T-S-D-Y-S-I-Aib-L-E-K-Q-A-Q-Aib-A-F-V-R-H-L-I-N-K(1,3)-Aib-T-R-Q-dOR-
Y-NH2.C18DA-gGlu-gGlu-gGlu-Glu-gGlu-Glu-eLys(1,2)

MW (calculated): 5290.0 Da
Synthesis and purification methods: S01; L20
LCMS: U046_001; Rt: 9.2 min; m/3: m/4: 1322.9

Compound 91
(SEQ ID NO 91)
Y-Aib-E-G-T-F-T-S-D-Y-S-I-Aib-L-E-K-Q-R-Q-Aib-A-F-V-R-H-L-I-N-K(1,3)-Aib-T-R-Q-R-Y-
NH2.C18DA-gGlu-gGlu-gGlu-gGlu-gGlu-Glu-eLys(1,2)

MW (calculated): 5388.1 Da
Synthesis and purification methods: S01; L20
LCMS: U046_001; Rt: 9.4 min; m/3: m/4: 1347.0

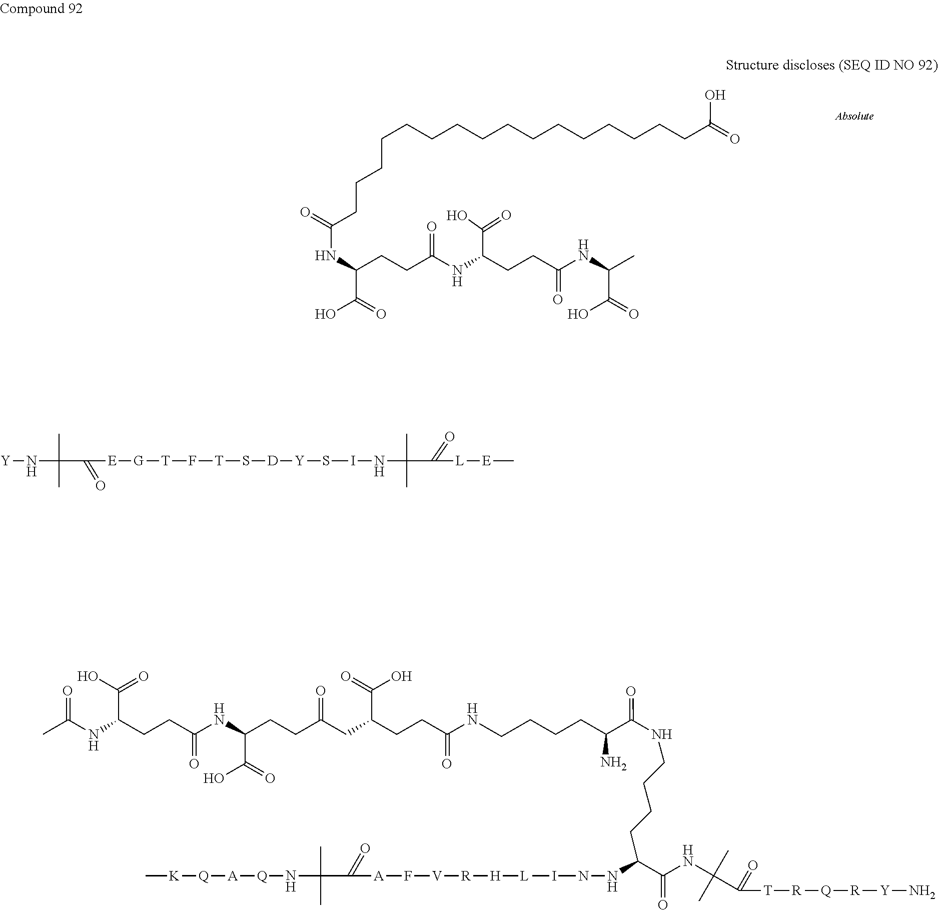

(SEQ ID NO 92)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5303.0 Da
Synthesis and purification methods: S01; L20
LCMS: U046_001; Rt: 9.5 min; m/3: m/4: 1326.6

Compound 93
(SEQ ID NO 93)
Y-Aib-E-A-T-F-T-S-D-Y-S-I-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5317.0 Da
Synthesis and purification methods: S01; L20
LCMS: U046_001; Rt: 9.6 min; m/3: m/4: 1330.0

Compound 94
(SEQ ID NO 94)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-eLys-gGlu(1,2)

MW (calculated): 5331.0 Da
Synthesis and purification methods: S01; L20
LCMS: U046_001; Rt: 11.4 min; m/3: m/4: 1333.6

Compound 95
(SEQ ID NO 95)
Y-Aib-E-G-T-F-T-S-D-Y-S-I-Aib-L-E-G-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5231.9 Da
Synthesis and purification methods: S01; L20
LCMS: U046_001; Rt: 11.9 min; m/3: m/4: 1308.7

Compound 96
(SEQ ID NO 96)
Y-Aib-E-G-T-F-T-S-D-Y-S-I-Aib-L-E-K-Q-A-Q-Aib-A-F-V-R-H-L-I-N-K(1,3)-Aib-T-R-Q-dOR-Y-
NH2.C18DA-gGlu-gGlu-gGlu-gGlu-gGlu(1,2)

MW (calculated): 5031.7 Da
Synthesis and purification methods: S01; L20
LCMS: U046_001; Rt: 9.9 min; m/3: m/4: 1258.7

Compound 97
(SEQ ID NO 97)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-dtOrn(1,2)

MW (calculated): 5317.0 Da
Synthesis and purification methods: S01; L20
LCMS: U046_001; Rt: 11.2 min; m/3: m/4: 1330.0

Compound 98
(SEQ ID NO 98)
Y-Aib-E-G-T-F-T-S-D-Y-S-I-Aib-L-E-K-Q-A-Q-Aib-E-F-V-R-H-L-I-N-K(1,3)-Aib-T-R-Q-R-
Y-NH2.C18DA-gGlu-gGlu-gGlu-gGlu-gGlu-OEG-OEG(1,2)

MW (calculated): 5394.0 Da
Synthesis and purification methods: S01; L20
LCMS: U046_001; Rt: 10 min; m/3: m/4: 1349.3

Compound 99
(SEQ ID NO 99)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-OEG(1,2)

MW (calculated): 5348.0 Da
Synthesis and purification methods: S01; L20
LCMS: U046_001; Rt: 11.7 min; m/3: m/4: 1337.8

Compound 100
(SEQ ID NO 100)
Y-Aib-E-G-T-F-T-S-D-Y-S-I-Aib-L-E-K-E-A-Q-Aib-E-F-V-R-H-L-I-N-K(1,3)-Aib-T-R-Q-R-Y-
NH2.C18DA-gGlu-gGlu-gGlu-Glu(1,2)

MW (calculated): 4975.6 Da
Synthesis and purification methods: S01; L20
LCMS: U046_001; Rt: 10.9 min; m/3: m/4: 1244.7

Compound 101
(SEQ ID NO 101)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-gGlu(1,2)

MW (calculated): 5332.0 Da
Synthesis and purification methods: S01; L20
LCMS: U046_001; Rt: 12 min; m/3: m/4: 1333.8

Compound 102
(SEQ ID NO 102)
Y-Aib-E-G-T-F-T-S-D-Y-S-I-Aib-L-D-K-Q-A-Q-Aib-A-F-I-R-H-L-I-N-K(1,3)-Aib-T-R-Q-R-Y-
NH2.C18DA-gGlu-gGlu-gGlu-gGlu-gGlu-gGlu-eLys(1,2)

MW (calculated): 5303.0 Da
Synthesis and purification methods: S01; L20
LCMS: U046_006; Rt: 30.7 min; m/3: m/4: 1326.5

Compound 103
(SEQ ID NO 103)
Y-Aib-E-G-T-F-T-S-D-Y-S-I-aMeL-L-E-K-Q-A-E-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5374.1 Da
Synthesis and purification methods: S01; L20
LCMS: U046_001; Rt: 11.7 min; m/3: m/4: 1344.3

Compound 104
(SEQ ID NO 104)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2)

MW (calculated): 5332.0 Da
Synthesis and purification methods: S01; L20
LCMS: U046_001; Rt: 11.4 min; m/3: m/4: 1333.8

Compound 105
(SEQ ID NO 105)
Y-Aib-E-G-T-F-T-S-D-Y-S-I-Aib-L-D-K-Q-A-Q-Aib-
A-F-V-R-H-L-I-N-K(1,3)-Aib-T-R-Q-R-Y-NH2.C18DA-
gGlu-gGlu-gGlu-gGlu-gGlu-gGlu-eLys(1,2)

MW (calculated): 5289.0 Da
Synthesis and purification methods: S01; L20
LCMS: U046_006; Rt: 30.5 min; m/3: m/4: 1323.0

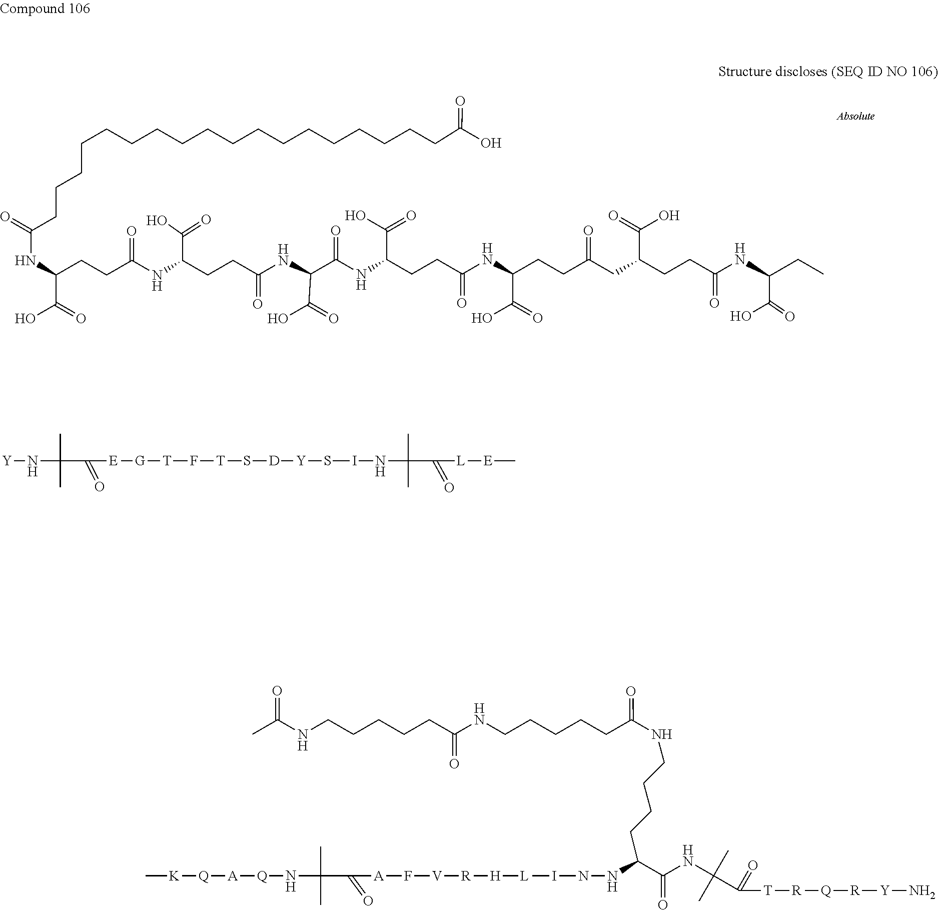

(SEQ ID NO 106)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C20DA-
gGlu-gGlu-gGlu-gGlu-gGlu-AHX-AHX(1,2)

MW (calculated): 5300.1 Da
Synthesis and purification methods: LP; L20
LCMS: U046_001; Rt: 11.9 min; m/3: m/4: 1325.8

Compound 107
(SEQ ID NO 107)
Y-Aib-E-G-T-F-T-S-D-Y-S-I-Aib-L-E-K-Q-A-Q-Aib-
A-F-V-R-H-L-L-N-K(1,3)-Aib-T-R-Q-R-Y-NH2.C18DA-
gGlu-gGlu-gGlu-gGlu-gGlu-gGlu-eLys(1,2)

MW (calculated): 5303.0 Da
Synthesis and purification methods: S01; L20
LCMS: U046_001; Rt: 9.5 min; m/3: m/4: 1326.5

Compound 108
(SEQ ID NO 108)
Y-Aib-E-G-T-F-T-S-D-Y-S-I-Aib-L-D-E-Q-A-Q-Aib-
A-F-V-R-H-L-I-N-K(1,3)-Aib-T-R-Q-R-Y-NH2.C18DA-
gGlu-gGlu-gGlu-gGlu-gGlu-eLys(1,2)

MW (calculated): 5160.8 Da
Synthesis and purification methods: S01; L20
LCMS: U046_006; Rt: 32.6 min; m/3: m/4: 1290.9

Compound 109
(SEQ ID NO 109)
Y-Aib-E-G-T-F-T-S-D-Y-S-I-Aib-L-E-K-Q-A-Q-Aib-
A-F-I-R-H-L-I-N-K(1,3)-Aib-T-R-Q-R-Y-NH2.C20DA-
gGlu-gGlu-gGlu-gGlu-gGlu-Glu-eLys(1,2)

MW (calculated): 5345.1 Da
Synthesis and purification methods: S01; L20
LCMS: U046_001; Rt: 11.4 min; m/3: m/4: 1337.0

Compound 110
(SEQ ID NO 110)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C18DA-
gGlu-Glu-gGlu-gGlu-OEG(1,2)

MW (calculated): 5061.7 Da
Synthesis and purification methods: S01; L20
LCMS: U046_001; Rt: 10.6 min; m/3: m/4: 1266.3

Compound 111
(SEQ ID NO 111)
Y-Aib-E-G-T-F-T-S-D-Y-S-I-Aib-L-E-K-Q-A-A-Aib-
A-F-V-R-H-L-I-N-K(1,3)-Aib-T-R-Q-R-Y-NH2.C18DA-
gGlu-gGlu-gGlu-gGlu-gGlu-gGlu-eLys(1,2)

MW (calculated): 5245.9 Da
Synthesis and purification methods: S01; L20
LCMS: U046_001; Rt: 11.5 min; m/3: m/4: 1312.2

Compound 112
(SEQ ID NO 112)
Y-Aib-E-G-T-F-T-S-D-Y-S-E-Aib-L-E-K-Q-A-Q-Aib-
A-F-V-R-H-L-I-N-K(1,3)-Aib-T-R-Q-R-Y-NH2.C18DA-
gGlu-gGlu-gGlu-gGlu-gGlu-gGlu-eLys(1,2)

MW (calculated): 5318.9 Da
Synthesis and purification methods: S01; L20
LCMS: U046_001; Rt: 9 min; m/3: m/4: 1330.5

Compound 113
(SEQ ID NO 113)
Y-Aib-E-G-T-F-T-S-D-Y-S-I-Aib-L-D-K-Q-A-Q-Aib-
E-F-V-R-H-L-I-N-K(1,3)-Aib-T-R-Q-R-Y-NH2.C18DA-
gGlu-gGlu-gGlu-gGlu-gGlu-eLys(1,2)

MW (calculated): 5217.9 Da
Synthesis and purification methods: S01; L20
LCMS: U046_006; Rt: 30.5 min; m/3: m/4: 1305.2

Compound 114
(SEQ ID NO 114)
Y-Aib-E-G-T-F-T-S-D-Y-S-I-Aib-L-E-K-Q-A-Q-Aib-A-
F-V-R-H-L-I-N-K(1,3)-Aib-T-R-NMeQ-R-Y-NH2.C20DA-
gGlu-gGlu-gGlu-gGlu-gGlu-gGlu(1,2)

MW (calculated): 5216.9 Da
Synthesis and purification methods: S01; L20
LCMS: U046_001; Rt: 12.2 min; m/3: m/4: 1305.0

Compound 115
(SEQ ID NO 115)
Y-Aib-E-G-T-F-T-S-D-L-S-I-Aib-L-E-K-Q-A-Q-Aib-
A-F-V-R-H-L-L-N-K(1,3)-Aib-T-R-Q-R-Y-NH2.C20DA-
gGlu-gGlu-gGlu-gGlu-gGlu-gGlu-eLys(1,2)

MW (calculated): 5281.0 Da
Synthesis and purification methods: S01; L20
LCMS: U046_001; Rt: 12 min; m/3: m/4: 1321.0

Compound 116
(SEQ ID NO 116)
Y-Aib-E-G-T-F-T-S-D-Y-S-I-Aib-L-E-K-Q-A-Q-Aib-A-
F-V-R-H-L-I-N-K(1,3)-Aib-T-R-Q-R-NMeY-NH2.C18DA-
gGlu-gGlu-gGlu-gGlu-gGlu-gGlu-eLys(1,2)

MW (calculated): 5317.0 Da
Synthesis and purification methods: S01; L20
LCMS: U046_001; Rt: 10 min; m/3: m/4: 1330.0

Compound 117
(SEQ ID NO 117)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C18DA-
gGlu-gGlu-gGlu-gGlu-Glu-OEG-OEG(1,2)

MW (calculated): 5336.0 Da
Synthesis and purification methods: S01; L20
LCMS: U046_001; Rt: 10.1 min; m/3: m/4: 1334.8

Compound 118
(SEQ ID NO 118)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C20DA-
TRX-gGlu-gGlu-Glu-gGlu-gGlu-Glu-eLys(1,2)

MW (calculated): 5470.2 Da
Synthesis and purification methods: S01; L20
LCMS: U046_001; Rt: 12.9 min; m/3: m/4: 1368.3

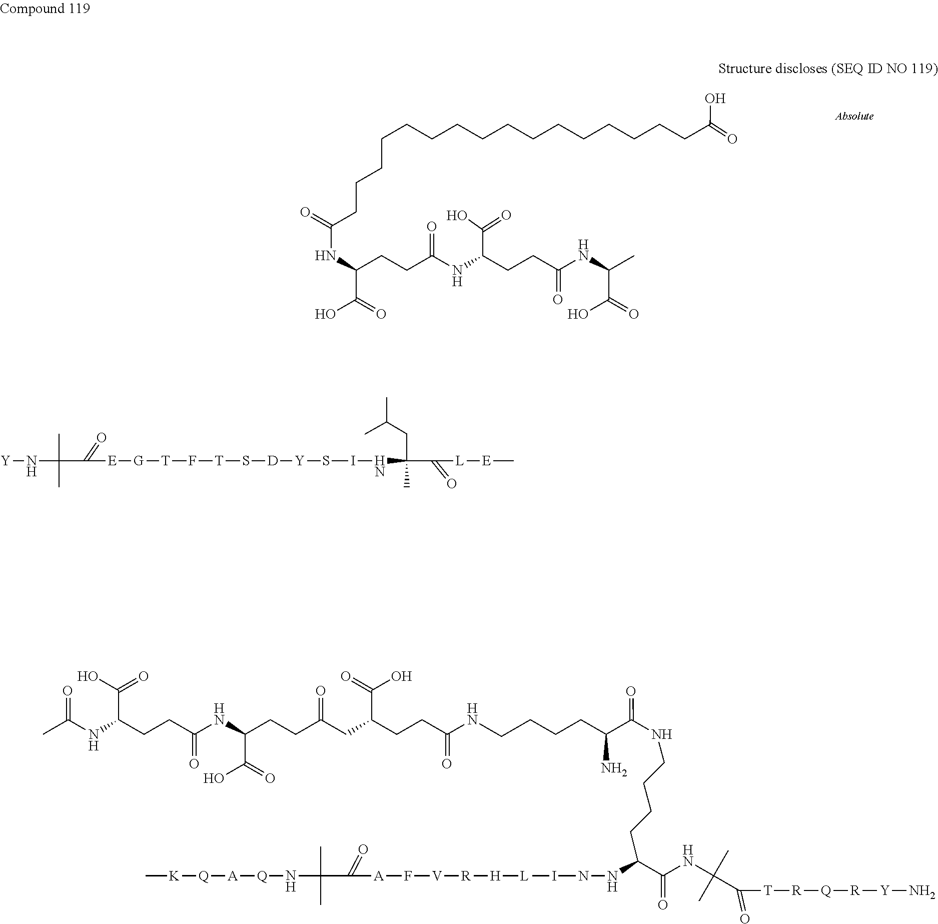

(SEQ ID NO 119)
Y-Aib-E-G-T-F-T-S-D-Y-S-I-aMeL-L-E-K-Q-A-Q-Aib-
A-F-V-R-H-L-I-N-K(1,3)-Aib-T-R-Q-R-Y-NH2.C18DA-
gGlu-gGlu-gGlu-gGlu-gGlu-gGlu-eLys(1,2)

MW (calculated): 5345.1 Da
Synthesis and purification methods: S01; L20
LCMS: U046_001; Rt: 9.9 min; m/3: m/4: 1337.1

Compound 120
(SEQ ID NO 120)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C18DA
-gGlu-gGlu-gGlu-gGlu-gGlu-gGlu-gDab(1,2)

MW (calculated): 5274.9 Da
Synthesis and purification methods: S01; L20
LCMS: U046_001; Rt: 9.6 min; m/3: m/4: 1319.6

Compound 121
(SEQ ID NO 121)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C18DA
-gGlu-gGlu-gGlu-gGlu-gGlu-gGlu-gGlu-eLys(1,2)

MW (calculated): 5432.1 Da
Synthesis and purification methods: S01; L20
LCMS: U046_001; Rt: 9.4 min; m/3: m/4: 1358.8

Compound 122
(SEQ ID NO 122)
Y-Aib-E-G-T-F-T-S-D-Y-S-I-L-L-E-K-Q-A-Q-Aib-A-
F-V-R-H-L-I-N-K(1,3)-Aib-T-R-Q-R-Y-NH2.C18DA-g
Glu-gGlu-gGlu-gGlu-gGlu(1,2)

MW (calculated): 5073.7 Da
Synthesis and purification methods: S01; L20
LCMS: U046_001; Rt: 10.8 min; m/3: m/4: 1269.2

Compound 123
(SEQ ID NO 123)
Y-Aib-E-G-T-F-T-S-D-Y-S-I-Aib-L-E-A-Q-A-Q-Aib-
A-F-V-R-H-L-I-N-K(1,3)-Aib-T-R-Q-R-Y-NH2.C18DA
-gGlu-gGlu-gGlu-gGlu-gGlu-gGlu-eLys(1,2)

MW (calculated): 5245.9 Da
Synthesis and purification methods: S01; L20
LCMS: U046_001; Rt: 12.3 min; m/3: m/4: 1312.3

Compound 124
(SEQ ID NO 124)
Y-Aib-E-G-T-F-T-S-D-Y-S-I-Aib-L-E-R-Q-A-Q-Aib-
A-F-V-R-H-L-I-N-K(1,3)-Aib-T-R-Q-R-Y-NH2.C18DA
-gGlu-gGlu-gGlu-Glu-gGlu-gGlu-eLys(1,2)

MW (calculated): 5331.0 Da
Synthesis and purification methods: S01; L20
LCMS: U046_006; Rt: 30.9 min; m/3: m/4: 1333.5

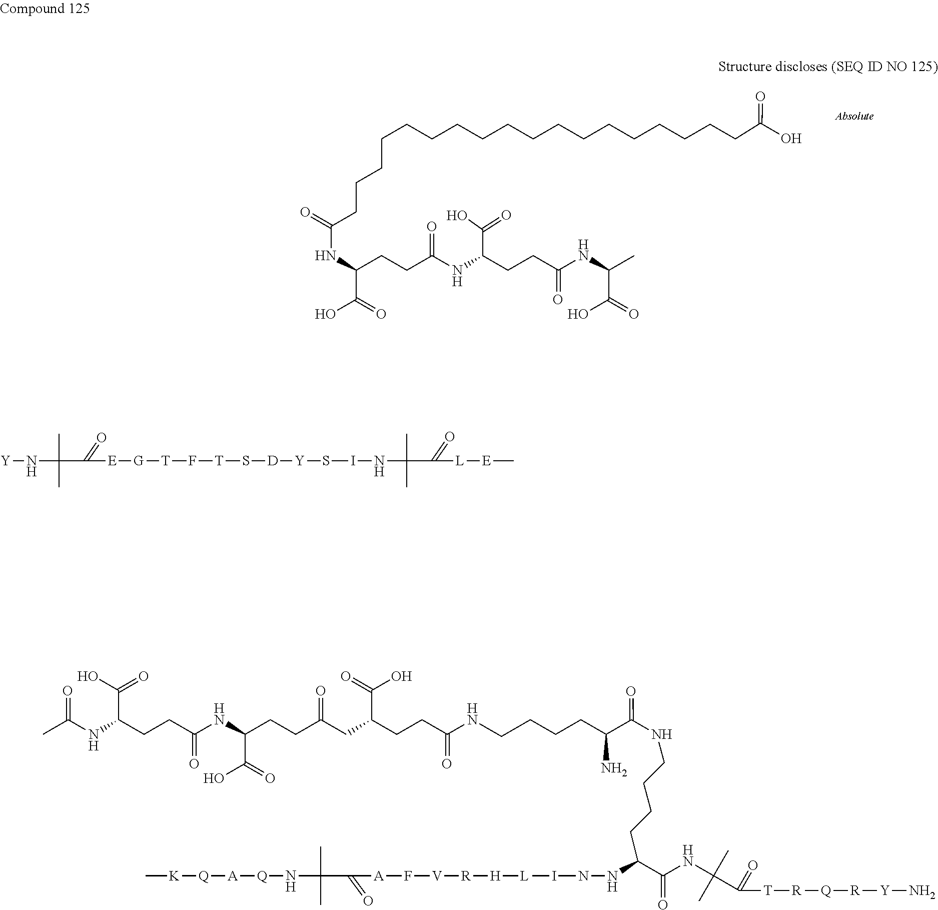

(SEQ ID NO 125)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C20DA
-gGlu-gGlu-gGlu-gGlu-gGlu-gGlu-eLys(1,2)

MW (calculated): 5331.0 Da
Synthesis and purification methods: S01; L20
LCMS: U046_001; Rt: 11.2 min; m/3: m/4: 1333.5

Compound 126
(SEQ ID NO 126)
Y-Aib-E-G-T-F-T-S-D-Y-S-I-Aib-L-E-K-Q-A-Q-Aib-
A-F-V-R-H-L-I-N-K(1,3)-V-T-R-Q-R-Y-NH2.C18DA-g
Glu-gGlu-gGlu-gGlu-gGlu-Glu-eLys(1,2)

MW (calculated): 5317.0 Da
Synthesis and purification methods: S01; L20
LCMS: 010_CA07; Rt: 9.4 min; m/3: m/4: 1330.5

Compound 127
(SEQ ID NO 127)
Y-Aib-E-G-T-F-T-S-D-Y-S-I-Aib-L-E-K-Q-A-Q-Aib-
A-F-V-R-H-L-I-N-K(1,3)-L-T-R-Q-R-Y-NH2.C18DA-g
Glu-gGlu-gGlu-gGlu-gGlu-gGlu-eLys(1,2)

MW (calculated): 5331.0 Da
Synthesis and purification methods: S01; L20
LCMS: U046_001; Rt: 10.5 min; m/3: m/4: 1333.5

Compound 128
(SEQ ID NO 128)
Y-Aib-E-G-T-F-T-S-D-Y-S-I-Aib-L-E-K-Q-A-Q-Aib-
A-F-V-R-A-L-I-N-K(1,3)-Aib-T-R-Q-R-Y-NH2.C18DA
-gGlu-gGlu-gGlu-gGlu-gGlu-gGlu-eLys(1,2)

MW (calculated): 5236.9 Da
Synthesis and purification methods: S01; L20
LCMS: U046_001; Rt: 9.9 min; m/3: m/4: 1310.0

Compound 129
(SEQ ID NO 129)
Y-Aib-E-G-T-F-T-S-D-Y-S-I-Aib-L-E-E-Q-A-Q-Aib-
E-F-V-R-H-L-I-N-K(1,3)-Aib-T-R-Q-R-Y-NH2.C18DA
-gGlu-gGlu-gGlu-gGlu(1,2)

MW (calculated): 4975.6 Da
Synthesis and purification methods: S01; L20
LCMS: U046_001; Rt: 12.2 min; m/3:1659.2 m/4:

Compound 130
(SEQ ID NO 130)
Y-Aib-E-G-T-F-T-S-D-Y-S-I-Aib-L-E-E-E-A-Q-Aib-
E-F-V-R-H-L-I-N-K(1,3)-Aib-T-R-Q-R-Y-NH2.C18DA
-gGlu-OEG-OEG(1,2)

MW (calculated): 4879.5 Da
Synthesis and purification methods: S01; L20
LCMS: U046_001; Rt: 13.3 min; m/3:1627.2 m/4:

Compound 131
(SEQ ID NO 131)
Y-Aib-E-G-T-F-T-S-D-Y-S-I-Aib-L-D-K-Q-A-Q-Aib-
E-F-V-R-H-L-I-N-K(1,3)-Aib-T-R-Q-R-Y-NH2.C18DA
-gGlu-gGlu-gGlu-gGlu-gGlu-gGlu-eLys(1,2)

MW (calculated): 5347.0 Da
Synthesis and purification methods: S01; L20
LCMS: U046_006; Rt: 30.4 min; m/3: m/4: 1337.5

Compound 132
(SEQ ID NO 132)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C20DA
-gGlu-gGlu-gGlu-eLys-gGlu-gGlu-gGlu(1,2)

MW (calculated): 5331.0 Da
Synthesis and purification methods: S01; L20
LCMS: U046_001; Rt: 11.4 min; m/3: m/4: 1333.5

Compound 133
(SEQ ID NO 133)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C20DA
-TRX-gGlu-gGlu-gGlu-gGlu-gGlu-gGlu(1,2)

MW (calculated): 5342.1 Da
Synthesis and purification methods: S01; L20
LCMS: U046_001; Rt: 13.4 min; m/3: m/4: 1336.3

Compound 134
(SEQ ID NO 134)
Y-Aib-E-G-T-F-T-S-D-Y-S-I-Aib-L-E-K-Q-A-Q-Aib-
A-F-V-R-H-L-I-N-K(1,3)-Aib-T-R-Q-R-Y-NH2.C18DA
-gGlu-gGlu-gGlu-gGlu-gGlu-gGlu(1,2)

MW (calculated): 5174.8 Da
Synthesis and purification methods: S01; L20
LCMS: U046_001; Rt: 10.3 min; m/3: m/4: 1294.5

Compound 135
(SEQ ID NO 135)
Y-Aib-E-G-T-F-T-S-D-L-S-I-Aib-L-E-K-Q-A-Q-Aib-A-F-V-R-H-L-I-N-K(1,3)-Aib-T-R-Q-R-
Y-NH2.C20DA-gGlu-gGlu-gGlu-gGlu-gGlu-gGlu-gGlu-eLys(1,2)

MW (calculated): 5410.1 Da
Synthesis and purification methods: LP; L20
LCMS: U046_001; Rt: 12 min; m/3:1804.1 m/4:

Compound 136
(SEQ ID NO 136)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Y-NH2.C20DA-gGlu-gGlu-gGlu-gGlu-OEG-gGlu-gGlu(1,2)

MW (calculated): 5348.0 Da
Synthesis and purification methods: S01; L20
LCMS: U046_001; Rt: 11.6 min; m/3: m/4: 1337.8

Compound 137
(SEQ ID NO 137)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Y-NH2.C18DA-TRX-gGlu-gGlu-gGlu-gGlu-gGlu-gGlu-eLys(1,2)

MW (calculated): 5442.2 Da
Synthesis and purification methods: S01; L20
LCMS: U046_001; Rt: 11 min; m/3: m/4: 1361.3

Compound 138
(SEQ ID NO 138)
Y-Aib-E-G-T-F-T-S-D-Y-S-I-Aib-L-D-E-Q-A-Q-Aib-A-F-V-R-H-L-I-N-K(1,3)-Aib-T-R-Q-R-
Y-NH2.C20DA-gGlu-gGlu-gGlu-gGlu-gGlu-eLys(1,2)

MW (calculated): 5188.8 Da
Synthesis and purification methods: S01; L20
LCMS: U046_006; Rt: 34.3 min; m/3: m/4: 1298.0

Compound 139
(SEQ ID NO 139)
Y-Aib-E-G-T-F-T-S-D-Y-S-I-Aib-L-E-K-Q-A-Q-Aib-A-F-V-R-H-A-I-N-K(1,3)-Aib-T-R-Q-R-
Y-NH2.C18DA-gGlu-gGlu-gGlu-gGlu-gGlu-gGlu-eLys(1,2)

MW (calculated): 5260.9 Da
Synthesis and purification methods: S01; L20
LCMS: U046_001; Rt: 8.8 min; m/3: m/4: 1316.0

Compound 140
(SEQ ID NO 140)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Y-NH2.C18DA-gGlu-gGlu-gGlu-gGlu-gGlu-OEG(1,2)

MW (calculated): 5190.8 Da
Synthesis and purification methods: S01; L20
LCMS: U046_001; Rt: 10.3 min; m/3: m/4: 1298.4

Compound 141
(SEQ ID NO 141)
Y-Aib-E-G-T-F-T-S-D-Y-S-I-Aib-L-E-K-Q-A-Q-Aib-A-F-V-R-H-L-I-N-K(1,3)-Aib-T-R-Q-R-
Y-NH2.C18DA-gGlu-gGlu-gGlu-gGlu-gGlu-eLys(1,2)

MW (calculated): 5173.9 Da
Synthesis and purification methods: S01; L20
LCMS: U046_001; Rt: 9.7 min; m/3: m/4: 1294.2

Compound 142
(SEQ ID NO 142)
Y-Aib-E-G-T-F-T-S-D-Y-A-I-Aib-L-E-K-Q-A-Q-Aib-A-F-V-R-H-L-I-N-K(1,3)-Aib-T-R-Q-R-
Y-NH2.C18DA-gGlu-gGlu-gGlu-gGlu-gGlu-gGlu-eLys(1,2)

MW (calculated): 5287.0 Da
Synthesis and purification methods: S01; L20
LCMS: U046_001; Rt: 9.9 min; m/3: m/4: 1322.6

Compound 143
(SEQ ID NO 143)
Y-Aib-E-G-T-F-T-S-D-Y-S-I-Aib-L-E-K-A-A-Q-Aib-A-F-V-R-H-L-I-N-K(1,3)-Aib-T-R-Q-R-
Y-NH2.C18DA-gGlu-gGlu-gGlu-gGlu-Glu-gGlu-eLys(1,2)

MW (calculated): 5245.9 Da
Synthesis and purification methods: S01; L20
LCMS: U046_001; Rt: 10.4 min; m/3: m/4: 1312.2

Compound 144
(SEQ ID NO 144)
Y-Aib-E-G-T-F-T-S-D-Y-S-I-Aib-L-E-K-Q-A-Q-Aib-S-F-V-R-H-L-I-N-K(1,3)-Aib-T-R-Q-R-
Y-NH2.C18DA-gGlu-gGlu-gGlu-gGlu-gGlu-gGlu-eLys(1,2)

MW (calculated): 5319.0 Da
Synthesis and purification methods: S01; L20
LCMS: U046_001; Rt: 9.4 min; m/3: m/4: 1330.5

Compound 145
(SEQ ID NO 145)
Y-Aib-E-G-T-F-T-S-D-Y-S-I-Aib-L-E-K-Q-A-Q-Aib-E-F-V-R-H-L-I-N-K(1,3)-Aib-T-R-Q-R-
Y-NH2.C18DA-gGlu-gGlu-gGlu-gGlu-gGlu-gGlu-eLys(1,2)

MW (calculated): 5361.0 Da
Synthesis and purification methods: S01; L20
LCMS: U046_001; Rt: 9.6 min; m/3: m/4: 1341.0

Compound 146
(SEQ ID NO 146)
Y-Aib-E-G-T-F-T-S-D-Y-S-I-Aib-L-E-K-Q-A-Q-Aib-A-F-V-R-H-L-I-N-K(1,3)-Aib-T-R-Q-
bhArg-Y-NH2.C18DA-gGlu-gGlu-gGlu-gGlu-gGlu-gGlu-eLys(1,2)

MW (calculated): 5317.0 Da
Synthesis and purification methods: S01; L20
LCMS: U046_001; Rt: 9.7 min; m/3: m/4: 1330.0

Compound 147
(SEQ ID NO 147)
Y-Aib-E-G-T-F-T-S-D-Y-S-I-Aib-L-D-R-Q-A-Q-Aib-A-F-V-R-H-L-I-N-K(1,3)-Aib-T-R-Q-R-
Y-NH2.C18DA-gGlu-gGlu-gGlu-gGlu-gGlu-gGlu-eLys(1,2)

MW (calculated): 5317.0 Da
Synthesis and purification methods: S01; L20
LCMS: U046_006; Rt: 30.9 min; m/3:1773.0 m/4:

Compound 148
(SEQ ID NO 148)
Y-Aib-E-G-T-F-T-S-D-Y-S-I-Aib-L-E-K-Q-A-E-Aib-A-F-V-R-H-L-I-N-K(1,3)-Aib-T-R-Q-R-
Y-NH2.C20DA-gGlu-gGlu-gGlu-gGlu-OEG-gGlu-eLys(1,2)

MW (calculated): 5349.0 Da
Synthesis and purification methods: S01; L20
LCMS: U046_001; Rt: 11.4 min; m/3: m/4: 1337.7

Compound 149
(SEQ ID NO 149)
Y-Aib-E-G-T-F-T-S-D-Y-S-I-Aib-L-E-K-Q-A-Q-Aib-E-F-V-R-H-L-I-N-K(1,3)-Aib-T-R-Q-R-
Y-NH2.C20DA-gGlu-gGlu-gGlu-gGlu-gGlu-Glu-eLys(1,2)

MW (calculated): 5389.1 Da
Synthesis and purification methods: S01; L20
LCMS: U046_001; Rt: 11.2 min; m/3: m/4: 1348.0

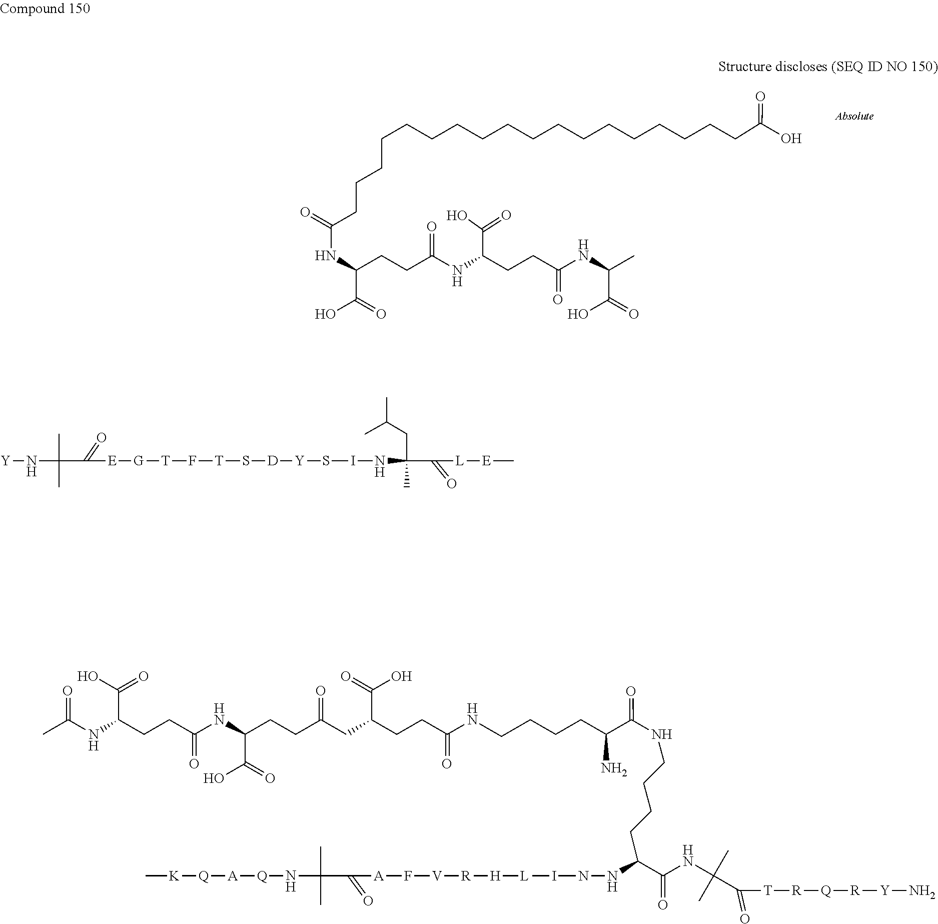

(SEQ ID NO 150)
Y-Aib-E-G-T-F-T-S-D-Y-S-I-aMeL-L-E-K-Q-A-Q-Aib-A-F-V-R-H-L-I-N-K(1,3)-Aib-T-R-
Q-R-Y-NH2.C20DA-gGlu-gGlu-gGlu-gGlu-gGlu-gGlu-eLys(1,2)

MW (calculated): 5373.1 Da
Synthesis and purification methods: S01; L20
LCMS: U046_001; Rt: 11.9 min; m/3: m/4: 1344.1

Compound 151
(SEQ ID NO 151)
Y-Aib-E-G-T-F-T-S-D-Y-S-I-Aib-L-E-K-Q-A-Q-Y-A-F-V-R-H-L-I-N-K(1,3)-Aib-T-R-Q-
R-Y-NH2.C18DA-gGlu-gGlu-gGlu-gGlu-gGlu-gGlu-eLys(1,2)

MW (calculated): 5381.1 Da
Synthesis and purification methods: S01; L20
LCMS: U046_001; Rt: 9.4 min; m/3: m/4: 1346.1

Compound 152
(SEQ ID NO 152)
Y-Aib-E-G-T-F-T-S-D-Y-S-I-Aib-L-E-K-Q-A-Q-Aib-E-F-V-R-H-L-I-N-K(1,3)-Aib-T-R-
Q-R-Y-NH2.C18DA-gGlu-gGlu-gGlu-gGlu-gGlu-gGlu-eLys-eLys(1,2)

MW (calculated): 5489.2 Da
Synthesis and purification methods: S01; L20
LCMS: U046_001; Rt: 9.2 min; m/3: m/4: 1373.1

Compound 153
(SEQ ID NO 153)
Y-Aib-E-G-T-F-T-S-D-Y-S-I-Aib-L-D-K-Q-A-Q-Aib-A-F-V-R-Y-L-I-N-K(1,3)-Aib-T-R-
Q-R-Y-NH2.C18DA-gGlu-gGlu-gGlu-gGlu-gGlu-gGlu-eLys(1,2)

MW (calculated): 5315.0 Da
Synthesis and purification methods: S01; L20
LCMS: U046_006; Rt: 31 min; m/3: m/4: 1329.5

Compound 154
(SEQ ID NO 154)
Y-Aib-E-G-T-F-T-S-D-Y-S-I-Aib-L-E-K-Q-A-Q-Aib-A-F-V-R-H-L-I-N-K(1,3)-Aib-T-R-
Q-R-Y-NH2.C18DA-gGlu-gGlu-gGlu-gGlu-gGlu-gGlu-gGlu-AHX(1,2)

MW (calculated): 5417.1 Da
Synthesis and purification methods: S01; L20
LCMS: U046_001; Rt: 10.1 min; m/3: m/4: 1355.1

Compound 155
(SEQ ID NO 155)
Y-Aib-E-G-T-F-T-S-D-Y-S-I-Aib-L-D-E-Q-A-Q-Aib-A-F-V-R-H-L-I-N-K(1,3)-Aib-T-R-
Q-R-Y-NH2.C20DA-gGlu-gGlu-gGlu-gGlu-gGlu-eLys-eLys(1,2)

MW (calculated): 5317.0 Da
Synthesis and purification methods: S01; L20
LCMS: U046_006; Rt: 34 min; m/3: m/4: 1330.0

Compound 156
(SEQ ID NO 156)
Y-Aib-E-G-T-F-T-S-D-Y-S-A-Aib-L-E-K-Q-A-Q-Aib-A-F-V-R-H-L-I-N-K(1,3)-Aib-T-R-
Q-R-Y-NH2.C18DA-gGlu-gGlu-gGlu-gGlu-gGlu-gGlu-eLys(1,2)

MW (calculated): 5260.9 Da
Synthesis and purification methods: S01; L20
LCMS: U046_001; Rt: 9.1 min; m/3: m/4: 1316.0

(SEQ ID NO 157)
Compound 157
Y-Aib-E-G-T-F-T-S-D-Y-S-I-Aib-L-E-K-Q-A-Q-Aib-A-F-V-R-H-L-I-N-K(1,3)-Aib-T-R-
Q-R-Y-NH2.C20DA-gGlu-gGlu-gGlu-gGlu-gGlu-gGlu-AHX(1,2)

MW (calculated): 5316.0 Da
Synthesis and purification methods: S01; L20
LCMS: U046_001; Rt: 11.7 min; m/3: m/4: 1329.8

Compound 158
(SEQ ID NO 158)
Y-Aib-E-G-T-F-T-S-D-Y-S-I-Aib-L-E-K-Q-A-Q-Aib-A-F-V-R-H-L-I-N-K(1,3)-L-T-R-Q-
R-Y-NH2.C18DA-gGlu-gGlu-gGlu-gGlu-gGlu(1,2)

MW (calculated): 5073.7 Da
Synthesis and purification methods: S01; L20
LCMS: U046_001; Rt: 11.5 min; m/3: m/4: 1269.2

Compound 159
(SEQ ID NO 159)
Y-Aib-E-G-T-F-T-S-D-Y-S-I-Aib-L-E-K-Q-A-Q-Aib-A-F-V-R-H-L-I-N-K(1,3)-A-T-R-
Q-R-Y-NH2.C18DA-gGlu-gGlu-gGlu-gGlu-gGlu-gGlu-eLys(1,2)

MW (calculated): 5289.0 Da
Synthesis and purification methods: S01; L20
LCMS: U046_001; Rt: 9.1 min; m/3: m/4: 1323.0

Compound 160
(SEQ ID NO 160)
Y-Aib-E-G-T-F-T-S-D-Y-S-I-E-L-E-K-Q-A-Q-Aib-A-F-V-R-H-L-I-N-K(1,3)-Aib-T-R-Q-
R-Y-NH2.C18DA-gGlu-gGlu-gGlu-gGlu-gGlu-gGlu-eLys(1,2)

MW (calculated): 5347.0 Da
Synthesis and purification methods: S01; L20
LCMS: U046_001; Rt: 9.4 min; m/3: m/4: 1337.5

Compound 161
(SEQ ID NO 161)
Y-Aib-E-G-T-F-T-S-D-Y-S-I-Aib-L-E-K-Q-A-E-Aib-A-F-V-R-H-L-I-N-K(1,3)-Aib-T-R-
Q-R-Y-NH2.C20DA-gGlu-gGlu-gGlu-gGlu-gGlu-OEG-eLys(1,2)

MW (calculated): 5349.0 Da
Synthesis and purification methods: S01; L20
LCMS: U046_001; Rt: 11.4 min; m/3: m/4: 1337.8

Compound 162
(SEQ ID NO 162)
Y-Aib-E-G-T-F-T-S-D-Y-S-I-Aib-L-E-K-Q-A-E-Aib-A-F-V-R-H-L-I-N-K(1,3)-Aib-T-R-
Q-R-Y-NH2.C20DA-gGlu-gGlu-gGlu-OEG-gGlu-gGlu-eLys(1,2)

MW (calculated): 5348.1 Da
Synthesis and purification methods: S01; L20
LCMS: U046_001; Rt: 11.4 min; m/3: m/4: 1337.9

Compound 163
(SEQ ID NO 163)
Y-Aib-E-G-T-F-T-S-D-Y-S-I-Aib-L-E-K-Q-A-Q-Aib-E-F-V-R-H-L-I-N-K(1,3)-Aib-T-R-
Q-R-Y-NH2.C18DA-gGlu-gGlu-gGlu-gGlu-eLys(1,2)

MW (calculated): 5102.8 Da
Synthesis and purification methods: S01; L20
LCMS: U046_001; Rt: 9.8 min; m/3: m/4: 1276.5

Compound 164
(SEQ ID NO 164)
Y-Aib-E-G-T-F-T-S-D-Y-S-I-Aib-L-E-K-E-A-Q-Aib-A-F-V-R-H-L-I-N-K(1,3)-Aib-T-R-
Q-R-Y-NH2.C18DA-gGlu-gGlu-gGlu-gGlu-gGlu-gGlu-eLys(1,2)

MW (calculated): 5304.0 Da
Synthesis and purification methods: S01; L20
LCMS: U046_001; Rt: 10 min; m/3: m/4: 1326.7

Compound 165
(SEQ ID NO 165)
Y-Aib-E-G-T-F-T-S-D-Y-S-I-A-L-E-K-Q-A-Q-Aib-A
-F-V-R-H-L-I-N-K(1,3)-Aib-T-R-Q-R-Y-NH2.C18DA
-gGlu-gGlu-gGlu-gGlu-gGlu-gGlu-eLys(1,2)

MW (calculated): 5289.0 Da
Synthesis and purification methods: S01; L20
LCMS: U046_001; Rt: 9.8 min; m/3: m/4: 1323.0

Compound 166
(SEQ ID NO 166)
Y-Aib-E-G-T-F-T-S-D-L-S-I-Aib-L-E-K-Q-A-Q-Aib
-A-F-V-R-H-L-I-N-K(1,3)-Aib-T-R-Q-R-Y-NH2.C20
DA-gGlu-gGlu-gGlu-gGlu-eLys-gGlu-gGlu(1,2)

MW (calculated): 5281.0 Da
Synthesis and purification methods: S01; L20
LCMS: U046_001; Rt: 12.1 min; m/3: m/4: 1321.2

Compound 167
(SEQ ID NO 167)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C20
DA-gGlu-gGlu-gGlu-gGlu-gGlu-AHX(1,2)

MW (calculated): 5186.9 Da
Synthesis and purification methods: S01; L20
LCMS: U046_001; Rt: 11.9 min; m/3: m/4: 1297.5

Compound 168
(SEQ ID NO 168)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C18
DA-gGlu-gGlu-gGlu-eLys-gGlu-gGlu-gGlu(1,2)

MW (calculated): 5303.0 Da
Synthesis and purification methods: S01; L20
LCMS: U046_001; Rt: 9.7 min; m/3: m/4: 1326.6

Compound 169
(SEQ ID NO 169)
Y-Aib-E-G-T-F-T-S-D-L-S-I-Aib-L-E-K-Q-A-Q-Aib
-A-F-V-R-H-L-I-N-K(1,3)-Aib-T-R-Q-R-Y-NH2.C18
DA-gGlu-gGlu-gGlu-gGlu-gGlu-gGlu-eLys(1,2)

MW (calculated): 5253.0 Da
Synthesis and purification methods: LP; L20
LCMS: U046_001; Rt: 10.5 min; m/3:1751.7 m/4:

Compound 170
(SEQ ID NO 170)
Y-Aib-E-G-T-F-T-S-D-Y-S-I-Aib-L-E-K-Q-A-Q-E-A
-F-V-R-H-L-I-N-K(1,3)-Aib-T-R-Q-R-Y-NH2.C18DA
-gGlu-gGlu-gGlu-gGlu-gGlu-gGlu-eLys(1,2)

MW (calculated): 5347.0 Da
Synthesis and purification methods: S01; L20
LCMS: U046_001; Rt: 9.2 min; m/3: m/4: 1337.5

Compound 171
(SEQ ID NO 171)
Y-Aib-E-G-T-F-T-S-D-Y-S-I-Aib-L-E-R-Q-A-Q-Aib
-A-F-V-R-H-L-I-N-K(1,3)-Aib-T-R-Q-R-Y-NH2.C20
DA-gGlu-gGlu-gGlu-gGlu-gGlu-gGlu-eLys(1,2)

MW (calculated): 5359.0 Da
Synthesis and purification methods: S01; L20
LCMS: U046_001; Rt: 11.5 min; m/3: m/4: 1340.5

Compound 172
(SEQ ID NO 172)
Y-Aib-E-G-T-F-T-S-D-Y-S-I-Aib-L-E-K-Q-A-Q-Aib
-A-F-V-R-H-L-I-N-K(1,3)-W-T-R-Q-R-Y-NH2.C18DA
-gGlu-gGlu-gGlu-gGlu-gGlu-gGlu-eLys(1,2)

MW (calculated): 5404.1 Da
Synthesis and purification methods: S01; L20
LCMS: U046_001; Rt: 10 min; m/3: m/4: 1351.9

Compound 173
(SEQ ID NO 173)
Y-Aib-E-G-T-F-T-S-D-Y-S-I-Aib-L-D-K-Q-A-Q-Aib
-A-F-V-R-H-L-L-N-K(1,3)-Aib-T-R-Q-R-Y-NH2.C18
DA-gGlu-gGlu-gGlu-gGlu-gGlu-gGlu-eLys(1,2)

MW (calculated): 5289.0 Da
Synthesis and purification methods: S01; L20
LCMS: U046_006; Rt: 30.4 min; m/3: m/4: 1322.9

Compound 174
(SEQ ID NO 174)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C18
DA-gGlu-gGlu-gGlu-gGlu-gGlu-eLys-gGlu(1,2)

MW (calculated): 5303.0 Da
Synthesis and purification methods: S01; L20
LCMS: U046_001; Rt: 9.6 min; m/3: m/4: 1326.5

Compound 175
(SEQ ID NO 175)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C20
DA-gGlu-gGlu-gGlu-gGlu-AHX-gGlu-AHX(1,2)

MW (calculated): 5300.1 Da
Synthesis and purification methods: S01; L20
LCMS: U046_001; Rt: 11.9 min; m/3: m/4: 1325.8

Compound 176
(SEQ ID NO 176)
Y-Aib-E-G-T-F-T-S-D-Y-S-I-Aib-L-E-K-Q-A-Q-Aib
-A-F-V-R-H-L-I-N-K(1,3)-Aib-T-R-Q-R-Y-NH2.C20
DA-gGlu-gGlu-gGlu-gGlu-gGlu(1,2)

MW (calculated): 5073.7 Da
Synthesis and purification methods: S01; L20
LCMS: U046_001; Rt: 11 min; m/3: m/4: 1269.2

1. A compound of formula (I), 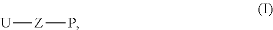
wherein P is a peptide comprising the amino acid sequence,
(SEQ ID NO: 185)
Y-Aib-E-X₄-X₅-F-T-S-D-X₁₀-X₁₁-X₁₂-X₁₃-L-X₁₅-X₁₆-
X₁₇-X₁₈-X₁₉-X₂₀-X₂₁-F-X₂₃-R-X₂₅-X₂₆-X₂₇-X₂₈-
K-X₃₀-T-R-X₃₃-X₃₄-X₃₅
wherein
X₄ is selected from the group consisting of A, and G,
X₅ is selected from the group consisting of A, E, and T,
X₁₀ is selected from the group consisting of F, L, and Y,
X₁₁ is selected from the group consisting of A, and S,
X₁₂ is selected from the group consisting of A, E, and I,
X₁₃ is selected from the group consisting of A, Aib, E, L, and aMeL,
X₁₅ is selected from the group consisting of D, and E,
X₁₆ is selected from the group consisting of A, Aib, E, G, K, L, LysAc, N, Q, and R,
X₁₇ is selected from the group consisting of A, E, I, and Q,
X₁₈ is selected from the group consisting of A, E and R,
X₁₉ is selected from the group consisting of A, E, Q, and Y,
X₂₀ is selected from the group consisting of A, Aib, E, and Y,
X₂₁ is selected from the group consisting of A, E, and S,
X₂₃ is selected from the group consisting of I, L, and V,
X₂₅ is selected from the group consisting of A, H, Q, and Y,
X₂₆ is selected from the group consisting of A, and L,
X₂₇ is selected from the group consisting of I, L, and Y,
X₂₈ is selected from the group consisting of A, and N,
X₃₀ is selected from the group consisting of A, Aib, I, L, V, and W,
X₃₃ is selected from the group consisting of NMeQ, and Q,
X₃₄ is selected from the group consisting of ArgMe, bhArg, dOR, NMeR, and R,
X₃₅ is selected from the group consisting of Phe4F, Y, and NMeY;
wherein U is a lipid moiety of formula (II) 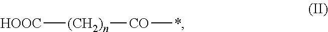
wherein n is an integer in the range 12 to 20,
wherein Z is a linker moiety comprising at least one [gGlu] residue,
wherein Z is linked to U on one end and to the epsilon-amino group of Lys at position-29 of the peptide P on the other end; and
wherein the compound of formula (I) comprises at least six residues of amino acids selected from the group consisting of E and [gGlu];
or salt thereof.

2. The compound according to claim 1,
wherein Z is a linker moiety consisting of three to ten linker sub-moieties covalently connected each other,
wherein the linker sub-moieties are independently selected from the group consisting of: absent, [gGlu], [eLys], [OEG], [AHX], [TRX], [gDab], and [dtOrn],
wherein [gGlu], [eLys], [OEG], [AHX], [TRX], [gDab], and [dtOrn] are residues of formula according to the following table

| | |
|---|---|
| [gGlu] | 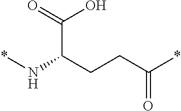 [SMILES: *N[C@@H](CCC(*)=O)C(=O)O] |
| [eLys] | 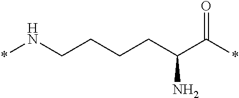 [SMILES: *NCCCC[C@H](N)C(*)=O] |
| [AHX] | 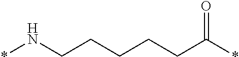 [SMILES: *NCCCCCC(*)=O] |
| [OEG] | 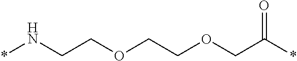 [SMILES: *NCCOCCOCC(*)=O] |
| [TRX] | 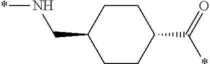 [SMILES: *NC[C@H]1CC[C@H](C(*)=O)CC1] |
| [gDab] | 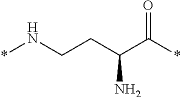 [SMILES: *NCC[C@H](N)C(*)=O] |
| [dtOrn] | 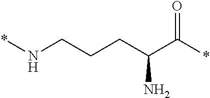 [SMILES: *NCCC[C@H](N)C(*)=O] |

and wherein the first linker sub-moiety of the linker Z is linked to U and the last linker sub-moiety on the other end of the linker Z is linked to the epsilon-amino group of Lys at position-29 of the peptide P;
preferably, wherein Z is a linker moiety consisting of three to eight linker sub-moieties.

3. The compound according to claim 1, wherein P is a peptide comprising the amino acid sequence
(SEQ ID NO: 186)
Y-Aib-E-X₄-X₅-F-T-S-D-X₁₀-X₁₁-X₁₂-X₁₃-L-X₁₅-X₁₆-
X₁₇-X₁₈-X₁₉-X₂₀-X₂₁-F-X₂₃-R-X₂₅-X₂₆-X₂₇-X₂₈-
K-X₃₀-T-R-X₃₃-X₃₄-X₃₅,
wherein
X₄ is selected from the group consisting of A, and G,
X₅ is T,
X₁₀ is selected from the group consisting of F, L, and Y,
X₁₁ is selected from the group consisting of A, and S,
X₁₂ is I,
X₁₃ is selected from the group consisting of Aib, and aMeL,
X₁₅ is selected from the group consisting of D, and E,
X₁₆ is selected from the group consisting of K, N, and E,
X₁₇ is selected from the group consisting of A, and Q,
X₁₈ is selected from the group consisting of A, and R,
X₁₉ is selected from the group consisting of A, E, and Q,
X₂₀ is selected from the group consisting of A, Aib, and Y,
X₂₁ is selected from the group consisting of A, and S,
X₂₃ is selected from the group consisting of I, L, and V,
X₂₅ is selected from the group consisting of A, H and Y,
X₂₆ is L,
X₂₇ is selected from the group consisting of I, and L,
X₂₈ is N,
X₃₀ is selected from the group consisting of A, Aib, I, L, and V,
X₃₃ is selected from the group consisting of NMeQ, and Q,
X₃₄ is selected from the group consisting of dOR, and R,
X₃₅ is selected from the group consisting of Phe4F, Y, and NMeY;
preferably, wherein P is a peptide comprising the amino acid sequence SEQ ID NO: 187.

4. The compound according to claim 1, wherein the C-terminal of peptide P is amidate.

5. The compound according to claim 1, wherein U is the lipid moiety of formula (II) 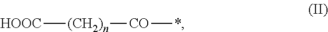
wherein n is an integer in the range 14 to 18;
preferably, wherein U is selected from the group consisting of 15-carboxy-pentadecanoyl (C16DA), 17-carboxy-heptadecanoyl (C18DA), and 19-carboxy-nonadecanoyl (C20DA).

6. The compound according to claim 1, wherein U is 17-carboxy-heptadecanoyl (C18DA).

7. The compound according to claim 1, wherein U is 19-carboxy-nonadecanoyl (C20DA).

8. The compound according to claim 1, wherein U-Z is selected from the group consisting of:
U-[gGlu]-[OEG]-[OEG],
U-[gGlu]-[gGlu]-[gGlu]-[OEG]-[gGlu]-[gGlu]-[eLys], U-[gGlu]-[gGlu]-[gGlu]-[eLys]-[gGlu]-[gGlu]-[gGlu],
U-[gGlu]-[gGlu]-[gGlu]-[gGlu], U-[gGlu]-[gGlu]-[gGlu]-[gGlu]-[OEG], U-[gGlu]-[gGlu]-[gGlu]-[gGlu]-[OEG]-[OEG], U-[gGlu]-[gGlu]-[gGlu]-[gGlu]-[eLys], U-[gGlu]-[gGlu]-[gGlu]-[gGlu]-[AHX]-[gGlu]-[AHX], U-[gGlu]-[gGlu]-[gGlu]-[gGlu]-[OEG]-[gGlu]-[eLys], U-[gGlu]-[gGlu]-[gGlu]-[gGlu]-[OEG]-[gGlu]-[gGlu], U-[gGlu]-[gGlu]-[gGlu]-[gGlu]-[eLys]-[gGlu]-[gGlu],
U-[gGlu]-[gGlu]-[gGlu]-[gGlu]-[gGlu], U-[gGlu]-[gGlu]-[gGlu]-[gGlu]-[gGlu]-[OEG], U-[gGlu]-[gGlu]-[gGlu]-[gGlu]-[gGlu]-[OEG]-[OEG], U-[gGlu]-[gGlu]-[gGlu]-[gGlu]-[gGlu]-[eLys], U-[gGlu]-[gGlu]-[gGlu]-[gGlu]-[gGlu]-[eLys]-[eLys], U-[gGlu]-[gGlu]-[gGlu]-[gGlu]-[gGlu]-[AHX], U-[gGlu]-[gGlu]-[gGlu]-[gGlu]-[gGlu]-[AHX]-[AHX], U-[gGlu]-[gGlu]-[gGlu]-[gGlu]-[gGlu]-[OEG]-[eLys], U-[gGlu]-[gGlu]-[gGlu]-[gGlu]-[gGlu]-[eLys]-[gGlu],
U-[gGlu]-[gGlu]-[gGlu]-[gGlu]-[gGlu]-[gGlu], U-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[OEG], U-[gGlu]-[gGlu]-[gGlu]-[gGlu]-[gGlu]-[gGlu]-[eLys]-[eLys], U-[gGlu]-[gGlu]-[gGlu]-[gGlu]-[gGlu]-[gGlu]-[AHX], U-[gGlu]-[gGlu]-[gGlu]-[gGlu]-[gGlu]-[gGlu]-[gDab], U-[gGlu]-[gGlu]-[gGlu]-[gGlu]-[gGlu]-[gGlu]-[dtOrn],
U-[gGlu]-[gGlu]-[gGlu]-[gGlu]-[gGlu]-[gGlu]-[gGlu], U-[gGlu]-[gGlu]-[gGlu]-[gGlu]-[gGlu]-[gGlu]-[gGlu]-[eLys], U-[gGlu]-[gGlu]-[gGlu]-[gGlu]-[gGlu]-[gGlu]-[gGlu]-[AHX],
U-[TRX]-[gGlu]-[gGlu]-[gGlu]-[gGlu]-[gGlu]-[gGlu], U-[TRX]-[gGlu]-[gGlu]-[gGlu]-[gGlu]-[gGlu]-[gGlu]-[eLys];
preferably, wherein U is 17-carboxy-heptadecanoyl (C18DA) or 19-carboxy-nonadecanoyl (C20DA).

9. The compound according to claim 1,
wherein Z comprises at least five [gGlu] residues.

10. The compound according to claim 1,
wherein Z consists of seven to eight linker sub-moieties covalently connected each other;
preferably, wherein the linker sub-moieties are independently selected from the group consisting of absent, [gGlu], [eLys], [OEG], and [AHX].

11. A compound or a pharmaceutically acceptable salt thereof, wherein the compound is selected from the group consisting of
(SEQ ID NO: 1)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-AHX-AHX(1,2);
(SEQ ID NO: 2)
Y-Aib-E-G-T-F-T-S-D-Y-S-I-Aib-L-E-K-Q-A-Q-Aib-A-F-V-R-H-L-I-N-K(1,3)-Aib-T-R-NMeQ-R-Y-
NH2.C18DA-gGlu-gGlu-gGlu-gGlu-Glu-gGlu(1,2);
(SEQ ID NO: 3)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu(1,2);
(SEQ ID NO: 4)
Y-Aib-E-G-T-F-T-S-D-Y-S-I-Aib-L-E-K-A-A-Q-Aib-A-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 5)
Y-Aib-E-G-T-F-T-S-D-Y-S-I-Aib-L-E-Aib-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2);
(SEQ ID NO: 6)
Y-Aib-E-G-T-F-T-S-D-Y-S-I-Aib-L-E-N-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 7)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C18DA-
gGlu-gGlu-gGlu-gGlu-gGlu-OEG(1,2);
(SEQ ID NO: 8)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-gGlu-OEG(1,2);
(SEQ ID NO: 9)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-AHX(1,2);
(SEQ ID NO: 10)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C18DA-
gGlu-gGlu-gGlu-Glu-gGlu-gGlu-eLys(1,2);
(SEQ ID NO: 11)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-Glu-gGlu-eLys-eLys(1,2);
(SEQ ID NO: 12)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C18DA-
gGlu-gGlu-gGlu-gGlu-gGlu-gGlu(1,2);
(SEQ ID NO: 13)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-Glu-gGlu-AHX(1,2);
(SEQ ID NO: 14)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-gGlu-AHX(1,2);
(SEQ ID NO: 15)
Y-Aib-E-G-T-F-T-S-D-F-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 16)
Y-Aib-E-G-T-F-T-S-D-Y-S-I-Aib-L-D-K-Q-A-Q-Aib-A-F-V-R-H-L-I-N-K(1,3)-Aib-T-R-Q-dOR-Y-
NH2.C20DA-gGlu-gGlu-gGlu-gGlu-gGlu-gGlu-gGlu-eLys(1,2);
(SEQ ID NO: 17)
Y-Aib-E-G-T-F-T-S-D-Y-S-I-Aib-L-E-K-Q-A-Q-Aib-A-F-V-R-H-L-I-N-K(1,3)-Aib-T-R-NMeQ-R-Y-
NH2.C18DA-gGlu-gGlu-gGlu-gGlu-gGlu-gGlu-eLys(1,2);
(SEQ ID NO: 18)
Y-Aib-E-G-T-F-T-S-D-Y-S-I-Aib-L-E-K-Q-A-Q-Aib-A-F-V-R-H-L-I-N-K(1,3)-I-T-R-Q-R-Y-NH2.C18DA-
gGlu-gGlu-gGlu-gGlu-gGlu-gGlu-gGlu-eLys(1,2);
(SEQ ID NO: 19)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-gGlu-OEG(1,2);
(SEQ ID NO: 20)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 21)
Y-Aib-E-G-T-F-T-S-D-L-S-I-Aib-L-E-K-Q-A-Q-Aib-A-F-I-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 22)
Y-Aib-E-G-T-F-T-S-D-Y-S-I-aMeL-L-D-K-Q-A-Q-Aib-A-F-I-R-H-L-I-N-K(1,3)-Aib-T-R-Q-R-Y-
NH2.C20DA-gGlu-gGlu-gGlu-gGlu-gGlu-gGlu-eLys(1,2);
(SEQ ID NO: 23)
Y-Aib-E-G-T-F-T-S-D-Y-S-I-Aib-L-E-Q-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 24)
Y-Aib-E-G-T-F-T-S-D-Y-S-I-Aib-L-E-K-I-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 25)
Y-Aib-E-G-T-F-T-S-D-Y-S-I-Aib-L-E-E-E-A-Q-Aib-A-F-V-R-H-L-I-N-K(1,3)-Aib-T-R-Q-R-Y-NH2.C18DA-
gGlu-gGlu-gGlu-gGlu(1,2);
(SEQ ID NO: 26)
Y-Aib-E-G-T-F-T-S-D-Y-S-I-Aib-L-E-K-Q-A-Q-Aib-A-F-V-R-H-L-I-N-K(1,3)-Aib-T-R-NMeQ-R-Y-
NH2.C20DA-gGlu-gGlu-gGlu-gGlu-gGlu-Glu-eLys(1,2);
(SEQ ID NO: 27)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C20DA-
gGlu-gGlu-gGlu-gGlu-gGlu-gGlu-eLys(1,2);
(SEQ ID NO: 28)
Y-Aib-E-G-T-F-T-S-D-Y-S-I-Aib-L-E-K-E-A-Q-Aib-A-F-V-R-H-L-I-N-K(1,3)-Aib-T-R-Q-R-Y-NH2.C20DA-
gGlu-gGlu-gGlu-gGlu-Glu-gGlu-eLys(1,2);
(SEQ ID NO: 29)
Y-Aib-E-G-T-F-T-S-D-Y-S-I-A-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu(1,2);
(SEQ ID NO: 30)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-Glu-gGlu-gGlu-gGlu-gGlu-eLys(1,2);
(SEQ ID NO: 31)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
TRX-gGlu-gGlu-gGlu-gGlu-gGlu-gGlu(1,2);
(SEQ ID NO: 32)
Y-Aib-E-G-T-F-T-S-D-Y-S-I-Aib-L-E-K-Q-A-Q-Aib-E-F-V-R-H-L-I-N-K(1,3)-L-T-R-Q-R-Y-NH2.C18DA-
gGlu-gGlu-gGlu-gGlu-gGlu-gGlu-Glu-eLys(1,2);
(SEQ ID NO: 33)
Y-Aib-E-G-T-F-T-S-D-Y-S-I-Aib-L-D-K-I-A-Q-Aib-A-F-V-R-H-L-I-N-K(1,3)-Aib-T-R-Q-R-Y-NH2.C18DA-
gGlu-glu-gGlu-gGlu-gGlu-gGlu-eLys(1,2);
(SEQ ID NO: 34)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-Glu-eLys-gGlu-gGlu(1,2);
(SEQ ID NO: 35)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-gGlu-eLys(1,2);
(SEQ ID NO: 36)
Y-Aib-E-G-T-F-T-S-D-Y-S-I-Aib-L-E-K-Q-E-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 37)
Y-Aib-E-G-T-F-T-S-D-Y-S-I-Aib-L-E-K-Q-A-Q-Aib-A-F-L-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 38)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-Glu(1,2);
(SEQ ID NO: 39)
Y-Aib-E-G-T-F-T-S-D-Y-S-I-Aib-L-E-K-Q-A-Q-Aib-E-F-V-R-H-L-I-N-K(1,3)-L-T-R-Q-R-Y-NH2.C20DA-
gGlu-gGlu-gGlu-Glu-gGlu-gGlu-eLys(1,2);
(SEQ ID NO: 40)
Y-Aib-E-G-T-F-T-S-D-Y-S-I-Aib-L-D-E-Q-A-Q-Aib-A-F-V-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 41)
Y-Aib-E-G-T-F-T-S-D-Y-S-I-Aib-L-E-K-Q-A-Q-Aib-A-F-V-R-H-L-I-N-K(1,3)-I-T-R-Q-R-Y-NH2.C18DA-
gGlu-gGlu-gGlu-gGlu-gGlu(1,2);
(SEQ ID NO: 42)
Y-Aib-E-G-T-F-T-S-D-Y-S-I-Aib-L-E-Q-Q-A-Q-Aib-A-F-V-R-H-L-I-N-K(1,3)-Aib-T-R-Q-R-Y-NH2.C18DA-
gGlu-gGlu-gGlu-gGlu-gGlu(1,2);
(SEQ ID NO: 43)
Y-Aib-E-G-T-F-T-S-D-Y-S-I-Aib-L-E-K-Q-A-Q-Aib-A-F-V-R-H-L-I-N-K(1,3)-Aib-T-R-Q-NMeR-Y-
NH2.C18DA-gGlu-gGlu-gGlu-gGlu-gGlu-gGlu-eLys(1,2);
(SEQ ID NO: 44)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-AHX-AHX(1,2);
(SEQ ID NO: 45)
Y-Aib-E-G-T-F-T-S-D-Y-S-I-Aib-L-E-K-Q-A-Q-Aib-E-F-V-R-H-L-I-N-K(1,3)-L-T-R-Q-R-Y-NH2.C18DA-
gGlu-gGlu-gGlu-gGlu-Glu-AHX(1,2);
(SEQ ID NO: 46)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-Glu-gGlu-gGlu-gDab(1,2);
(SEQ ID NO: 47)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-OEG(1,2);
(SEQ ID NO: 48)
Y-Aib-E-G-T-F-T-S-D-Y-S-I-Aib-L-E-E-Q-A-Q-Aib-A-F-V-R-H-L-I-N-K(1,3)-Aib-T-R-Q-R-Y-NH2.C18DA-
gGlu-gGlu-gGlu-gGlu-gGlu-eLys(1,2);
(SEQ ID NO: 49)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu(1,2);
(SEQ ID NO: 50)
Y-Aib-E-G-T-F-T-S-D-Y-S-I-Aib-L-E-K-Q-A-Q-Aib-A-F-V-R-Q-L-I-N-K(1,3)-Aib-T-R-Q-R-Y-NH2.C18DA-
gGlu-gGlu-gGlu-gGlu-gGlu(1,2);
(SEQ ID NO: 51)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu(1,2);
(SEQ ID NO: 52)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-eLys-gGlu-gGlu(1,2);
(SEQ ID NO: 53)
Y-Aib-E-G-T-F-T-S-D-Y-S-I-aMeL-L-E-K-Q-A-Q-Aib-A-F-I-R-H-L-I-N-K(1,3)-Aib-T-R-Q-R-Y-H2.C20DA-
gGlu-gGlu-gGlu-gGlu-gGlu-gGlu-eLys(1,2);
(SEQ ID NO: 54)
Y-Aib-E-G-E-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 55)
Y-Aib-E-G-T-F-T-S-D-Y-S-I-Aib-L-D-A-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 56)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-gGlu-gGlu(1,2);
(SEQ ID NO: 57)
Y-Aib-E-G-T-F-T-S-D-Y-S-I-L-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-Glu(1,2);
(SEQ ID NO: 58)
Y-Aib-E-G-T-F-T-S-D-Y-S-I-Aib-L-E-K-Q-A-Q-Aib-A-F-V-R-H-L-Y-N-K(1,3)-Aib-T-R-Q-R-Y-NH2.C18DA-
gGlu-gGlu-gGlu-gGlu-gGlu-gGlu-eLys(1,2);
(SEQ ID NO: 59)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-eLys(1,2);
(SEQ ID NO: 60)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-eLys-gGlu(1,2);
(SEQ ID NO: 61)
Y-Aib-E-G-T-F-T-S-D-Y-S-I-Aib-L-E-K-Q-A-Q-A-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 62)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu(1,2);
(SEQ ID NO: 63)
Y-Aib-E-G-T-F-T-S-D-Y-S-I-Aib-L-E-LysAc-Q-A-Q-Aib-A-F-V-R-H-L-I-N-K(1,3)-Aib-T-R-Q-R-Y-
NH2.C18DA-gGlu-gGlu-gGlu-gGlu(1,2);
(SEQ ID NO: 64)
Y-Aib-E-G-T-F-T-S-D-Y-S-I-Aib-L-E-E-E-A-Q-Aib-E-F-V-R-H-L-I-N-K(1,3)-Aib-T-R-Q-R-Y-NH2.C18DA-
gGlu-gGlu-gGlu-gGlu(1,2);
(SEQ ID NO: 65)
Y-Aib-E-G-T-F-T-S-D-Y-S-I-Aib-L-E-L-Q-A-Q-Aib-A-F-V-R-H-L-I-N-K(1,3)-Aib-T-R-Q-R-Y-NH2.C18DA-
gGlu-gGlu-gGlu-Glu-Glu-gGlu-eLys(1,2);
(SEQ ID NO: 66)
Y-Aib-E-G-T-F-T-S-D-Y-S-I-Aib-L-E-K-Q-A-Q-Aib-A-F-V-R-H-L-I-N-K(1,3)-I-T-R-Q-R-Y-NH2.C18DA-
gGlu-gGlu-gGlu-gGlu-gGlu-gGlu-eLys(1,2);
(SEQ ID NO: 67)
Y-Aib-E-G-T-F-T-S-D-Y-S-I-aMeL-L-D-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2);
(SEQ ID NO: 68)
Y-Aib-E-G-T-F-T-S-D-Y-S-I-Aib-L-E-K-Q-A-Q-Aib-A-F-V-R-H-L-I-A-K(1,3)-Aib-T-R-Q-R-Y-NH2.C18DA-
gGlu-gGlu-gGlu-gGlu-gGlu-gGlu-eLys(1,2);
(SEQ ID NO: 69)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C18DA-
gGlu-gGlu-gGlu-gGlu-gGlu-AHX(1,2);
(SEQ ID NO: 70)
Y-Aib-E-G-T-F-T-S-D-Y-S-I-Aib-L-D-Q-Q-A-Q-Aib-A-F-V-R-H-L-I-N-K(1,3)-Aib-T-R-Q-R-Y-
NH2.C18DA-gGlu-gGlu-gGlu-gGlu-gGlu-eLys(1,2);
(SEQ ID NO: 71)
Y-Aib-E-G-T-F-T-S-D-Y-S-I-Aib-L-E-E-Q-A-Q-Aib-A-F-V-R-H-L-I-N-K(1,3)-Aib-T-R-Q-R-Y-NH2.C18DA-
gGlu-gGlu-gGlu-Glu-gGlu-gGlu-eLys(1,2);
(SEQ ID NO: 72)
Y-Aib-E-G-T-F-T-S-D-Y-S-I-Aib-L-E-K-Q-A-Q-Aib-A-F-V-R-H-L-I-N-K(1,3)-Aib-T-R-Q-ArgMe-Y-
NH2.C18DA-gGlu-gGlu-gGlu-gGlu-gGlu-gGlu-eLys(1,2);
(SEQ ID NO: 73)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-OEG-OEG(1,2);
(SEQ ID NO: 74)
Y-Aib-E-G-T-F-T-S-D-Y-S-I-Aib-L-D-K-Q-A-Q-Aib-A-F-V-R-H-L-I-N-K(1,3)-Aib-T-R-Q-R-Y-NH2.C20DA-
gGlu-Glu-gGlu-gGlu-gGlu-gGlu-eLys(1,2);
(SEQ ID NO: 75)
Y-Aib-E-G-T-F-T-S-D-Y-S-I-Aib-L-E-E-Q-A-Q-Aib-A-F-V-R-H-L-I-N-K(1,3)-Aib-T-R-Q-R-Y-NH2.C18DA-
gGlu-gGlu-gGlu-gGlu-gGlu(1,2);
(SEQ ID NO: 76)
Y-Aib-E-G-T-F-T-S-D-Y-S-I-aMeL-L-E-K-Q-A-Q-Aib-A-F-I-R-H-L-I-N-K(1,3)-Aib-T-R-Q-R-Y-
NH2.C18DA-gGlu-gGlu-gGlu-gGlu-gGlu-gGlu-eLys(1,2);
(SEQ ID NO: 77)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu(1,2);
(SEQ ID NO: 78)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-gGlu-eLys(1,2);
(SEQ ID NO: 79)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-Glu-gGlu-gGlu(1,2);
(SEQ ID NO: 80)
Y-Aib-E-G-T-F-T-S-D-Y-S-I-Aib-L-E-K-Q-A-Q-Aib-A-F-V-R-H-L-L-N-K(1,3)-Aib-T-R-Q-R-Y-NH2.C20DA-
gGlu-gGlu-gGlu-gGlu-gGlu-gGlu-eLys(1,2);
(SEQ ID NO: 81)
Y-Aib-E-G-T-F-T-S-D-Y-S-I-Aib-L-D-K-Q-A-Q-Aib-A-F-I-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 82)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C20DA-
gGlu-gGlu-glu-gGlu-gGlu-gGlu-eLys-eLys(1,2);
(SEQ ID NO: 83)
Y-Aib-E-G-T-F-T-S-D-Y-S-I-Aib-L-E-K-Q-A-Q-Aib-E-F-V-R-H-L-I-N-K(1,3)-L-T-R-Q-R-Y-NH2.C18DA-
gGlu-gGlu-gGlu-gGlu-gGlu-gGlu-eLys(1,2);
(SEQ ID NO: 84)
Y-Aib-E-G-T-F-T-S-D-Y-S-I-Aib-L-E-K-Q-A-Y-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 85)
Y-Aib-E-G-A-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 86)
Y-Aib-E-G-T-F-T-S-D-Y-S-I-Aib-L-E-K-Q-A-Q-Aib-A-F-V-R-H-L-I-N-K(1,3)-Aib-T-R-Q-R-Phe4F-
NH2.C18DA-gGlu-gGlu-gGlu-gGlu-gGlu-gGlu-eLys(1,2);
(SEQ ID NO: 87)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-dtOrn(1,2);
(SEQ ID NO: 88)
Y-Aib-E-G-T-F-T-S-D-Y-S-I-Aib-L-E-K-Q-A-Q-Aib-A-F-I-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 89)
Y-Aib-E-G-T-F-T-S-D-Y-S-I-aMeL-L-E-E-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2);
(SEQ ID NO: 90)
Y-Aib-E-G-T-F-T-S-D-Y-S-I-Aib-L-E-K-Q-A-Q-Aib-A-F-V-R-H-L-I-N-K(1,3)-Aib-T-R-Q-dOR-Y-
NH2.C18DA-gGlu-Glu-glu-gGlu-gGlu-gGlu-eLys(1,2);
(SEQ ID NO: 91)
Y-Aib-E-G-T-F-T-S-D-Y-S-I-Aib-L-E-K-Q-R-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 92)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 93)
Y-Aib-E-A-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 94)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-eLys-gGlu(1,2);
(SEQ ID NO: 95)
Y-Aib-E-G-T-F-T-S-D-Y-S-I-Aib-L-E-G-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 96)
Y-Aib-E-G-T-F-T-S-D-Y-S-I-Aib-L-E-K-Q-A-Q-Aib-A-F-V-R-H-L-I-N-K(1,3)-Aib-T-R-Q-dOR-Y-
NH2.C18DA-gGlu-gGlu-gGlu-gGlu-gGlu(1,2);
(SEQ ID NO: 97)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-dtOrn(1,2);
(SEQ ID NO: 98)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-Glu-Glu-Glu-OEG-OEG(1,2);
(SEQ ID NO: 99)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-OEG(1,2);
(SEQ ID NO: 100)
Y-Aib-E-G-T-F-T-S-D-Y-S-I-Aib-L-E-K-E-A-Q-Aib-E-F-V-R-H-L-I-N-K(1,3)-Aib-T-R-Q-R-Y-NH2.C18DA-
gGlu-gGlu-gGlu-gGlu(1,2);
(SEQ ID NO: 101)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-gGlu(1,2);
(SEQ ID NO: 102)
Y-Aib-E-G-T-F-T-S-D-Y-S-I-Aib-L-D-K-Q-A-Q-Aib-A-F-I-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 103)
Y-Aib-E-G-T-F-T-S-D-Y-S-I-aMeL-L-E-K-Q-A-E-Aib-A-F-V-R-H-L-I-N-K(1,3)-Aib-T-R-Q-R-Y-
NH2.C20DA-gGlu-gGlu-gGlu-gGlu-gGlu-Glu-eLys(1,2);
(SEQ ID NO: 104)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C20DA-
gGlu-gGlu-gGlu-gGlu-Glu-gGlu-eLys(1,2);
(SEQ ID NO: 105)
Y-Aib-E-G-T-F-T-S-D-Y-S-I-Aib-L-D-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 106)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-AHX-AHX(1,2);
(SEQ ID NO: 107)
Y-Aib-E-G-T-F-T-S-D-Y-S-I-Aib-L-E-K-Q-A-Q-Aib-A-F-V-R-H-L-L-N-K(1,3)-Aib-T-R-Q-R-Y-NH2.C18DA-
gGlu-gGlu-gGlu-gGlu-gGlu-gGlu-eLys(1,2);
(SEQ ID NO: 108)
Y-Aib-E-G-T-F-T-S-D-Y-S-I-Aib-L-D-E-Q-A-Q-Aib-A-F-V-R-H-L-I-N-K(1,3)-Aib-T-R-Q-R-Y-NH2.C18DA-
gGlu-gGlu-gGlu-gGlu-gGlu-eLys(1,2);
(SEQ ID NO: 109)
Y-Aib-E-G-T-F-T-S-D-Y-S-I-Aib-L-E-K-Q-A-Q-Aib-A-F-I-R-H-L-I-N-K(1,3)-Aib-T-R-Q-R-Y-NH2.C20DA-
gGlu-gGlu-Glu-gGlu-gGlu-gGlu-eLys(1,2);
(SEQ ID NO: 110)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-OEG(1,2);
(SEQ ID NO: 111)
Y-Aib-E-G-T-F-T-S-D-Y-S-I-Aib-L-E-K-Q-A-A-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 112)
Y-Aib-E-G-T-F-T-S-D-Y-S-E-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 113)
Y-Aib-E-G-T-F-T-S-D-Y-S-I-Aib-L-D-K-Q-A-Q-Aib-E-F-V-R-H-L-I-N-K(1,3)-Aib-T-R-Q-R-Y-NH2.C18DA-
gGlu-gGlu-gGlu-gGlu-Glu-eLys(1,2);
(SEQ ID NO: 114)
Y-Aib-E-G-T-F-T-S-D-Y-S-I-Aib-L-E-K-Q-A-Q-Aib-A-F-V-R-H-L-I-N-K(1,3)-Aib-T-R-NMeQ-R-Y-
NH2.C20DA-gGlu-gGlu-gGlu-gGlu-gGlu-gGlu(1,2);
(SEQ ID NO: 115)
Y-Aib-E-G-T-F-T-S-D-L-S-I-Aib-L-E-K-Q-A-Q-Aib-A-F-V-R-H-L-L-N-K(1,3)-Aib-T-R-Q-R-Y-NH2.C20DA-
gGlu-gGlu-gGlu-gGlu-gGlu-gGlu-eLys(1,2);
(SEQ ID NO: 116)
Y-Aib-E-G-T-F-T-S-D-Y-S-I-Aib-L-E-K-Q-A-Q-Aib-A-F-V-R-H-L-I-N-K(1,3)-Aib-T-R-Q-R-NMeY-
NH2.C18DA-gGlu-gGlu-gGlu-Glu-gGlu-gGlu-eLys(1,2);
(SEQ ID NO: 117)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-OEG-OEG(1,2);
(SEQ ID NO: 118)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
TRX-gGlu-gGlu-gGlu-gGlu-gGlu-gGlu-eLys(1,2);
(SEQ ID NO: 119)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C18DA-gGlu-gGlu-gGlu-gGlu-Glu-Glu-eLys(1,2);
(SEQ ID NO: 120)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-Glu-gGlu-gGlu-gGlu-gDab(1,2);
(SEQ ID NO: 121)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-Glu-gGlu-Glu-gGlu-gGlu-eLys(1,2);
(SEQ ID NO: 122)
Y-Aib-E-G-T-F-T-S-D-Y-S-I-L-L-E-K-Q-A-Q-Aib-A-F-V-R-H-L-I-N-K(1,3)-Aib-T-R-Q-R-Y-NH2.C18DA-
gGlu-gGlu-Glu-gGlu-gGlu(1,2);
(SEQ ID NO: 123)
Y-Aib-E-G-T-F-T-S-D-Y-S-I-Aib-L-E-A-Q-A-Q-Aib-A-F-V-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 124)
Y-Aib-E-G-T-F-T-S-D-Y-S-I-Aib-L-E-R-Q-A-Q-Aib-A-F-V-R-H-L-I-N-K(1,3)-Aib-T-R-Q-R-Y-NH2.C18DA-
gGlu-gGlu-Glu-gGlu-gGlu-gGlu-eLys(1,2);
(SEQ ID NO: 125)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 126)
Y-Aib-E-G-T-F-T-S-D-Y-S-I-Aib-L-E-K-Q-A-Q-Aib-A-F-V-R-H-L-I-N-K(1,3)-V-T-R-Q-R-Y-NH2.C18DA-
gGlu-gGlu-gGlu-gGlu-gGlu-gGlu-eLys(1,2);
(SEQ ID NO: 127)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C18DA-
gGlu-gGlu-gGlu-gGlu-gGlu-Glu-eLys(1,2);
(SEQ ID NO: 128)
Y-Aib-E-G-T-F-T-S-D-Y-S-I-Aib-L-E-K-Q-A-Q-Aib-A-F-V-R-A-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 129)
Y-Aib-E-G-T-F-T-S-D-Y-S-I-Aib-L-E-E-Q-A-Q-Aib-E-F-V-R-H-L-I-N-K(1,3)-Aib-T-R-Q-R-Y-NH2.C18DA-
gGlu-gGlu-gGlu-gGlu(1,2);
(SEQ ID NO: 130)
Y-Aib-E-G-T-F-T-S-D-Y-S-I-Aib-L-E-E-E-A-Q-Aib-E-F-V-R-H-L-I-N-K(1,3)-Aib-T-R-Q-R-Y-NH2.C18DA-
gGlu-OEG-OEG(1,2);
(SEQ ID NO: 131)
Y-Aib-E-G-T-F-T-S-D-Y-S-I-Aib-L-D-K-Q-A-Q-Aib-E-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 132)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-eLys-gGlu-gGlu-Glu(1,2);
(SEQ ID NO: 133)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
TRX-gGlu-gGlu-gGlu-gGlu-gGlu-gGlu(1,2);
(SEQ ID NO: 134)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-Glu-gGlu-Glu-gGlu(1,2);
(SEQ ID NO: 135)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-Glu-gGlu-gGlu-eLys(1,2);
(SEQ ID NO: 136)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-OEG-gGlu-gGlu(1,2);
(SEQ ID NO: 137)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
TRX-gGlu-gGlu-gGlu-Glu-gGlu-gGlu-eLys(1,2);
(SEQ ID NO: 138)
Y-Aib-E-G-T-F-T-S-D-Y-S-I-Aib-L-D-E-Q-A-Q-Aib-A-F-V-R-H-L-I-N-K(1,3)-Aib-T-R-Q-R-Y-NH2.C20DA-
gGlu-gGlu-gGlu-gGlu-gGlu-eLys(1,2);
(SEQ ID NO: 139)
Y-Aib-E-G-T-F-T-S-D-Y-S-I-Aib-L-E-K-Q-A-Q-Aib-A-F-V-R-H-A-I-N-K(1,3)-Aib-T-R-Q-R-Y-NH2.C18DA-
gGlu-gGlu-gGlu-gGlu-gGlu-gGlu-eLys(1,2);
(SEQ ID NO: 140)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-OEG(1,2);
(SEQ ID NO: 141)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-eLys(1,2);
(SEQ ID NO: 142)
Y-Aib-E-G-T-F-T-S-D-Y-A-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 143)
Y-Aib-E-G-T-F-T-S-D-Y-S-I-Aib-L-E-K-A-A-Q-Aib-A-F-V-R-H-L-I-N-K(1,3)-Aib-T-R-Q-R-Y-NH2.C18DA-
gGlu-gGlu-gGlu-Glu-gGlu-gGlu-eLys(1,2);
(SEQ ID NO: 144)
Y-Aib-E-G-T-F-T-S-D-Y-S-I-Aib-L-E-K-Q-A-Q-Aib-S-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 145)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-Glu-gGlu-gGlu-gGlu-eLys(1,2);
(SEQ ID NO: 146)
Y-Aib-E-G-T-F-T-S-D-Y-S-I-Aib-L-E-K-Q-A-Q-Aib-A-F-V-R-H-L-I-N-K(1,3)-Aib-T-R-Q-bhArg-Y-
NH2.C18DA-gGlu-gGlu-gGlu-gGlu-gGlu-gGlu-eLys(1,2);
(SEQ ID NO: 147)
Y-Aib-E-G-T-F-T-S-D-Y-S-I-Aib-L-D-R-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 148)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C20DA-
gGlu-gGlu-gGlu-gGlu-OEG-gGlu-eLys(1,2);
(SEQ ID NO: 149)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 150)
Y-Aib-E-G-T-F-T-S-D-Y-S-I-aMeL-L-E-K-Q-A-Q-Aib-A-F-V-R-H-L-I-N-K(1,3)-Aib-T-R-Q-R-Y-
NH2.C20DA-gGlu-gGlu-gGlu-gGlu-gGlu-gGlu-eLys(1,2);
(SEQ ID NO: 151)
Y-Aib-E-G-T-F-T-S-D-Y-S-I-Aib-L-E-K-Q-A-Q-Y-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 152)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-gGlu-gGlu-eLys-eLys(1,2);
(SEQ ID NO: 153)
Y-Aib-E-G-T-F-T-S-D-Y-S-I-Aib-L-D-K-Q-A-Q-Aib-A-F-V-R-Y-L-I-N-K(1,3)-Aib-T-R-Q-R-Y-NH2.C18DA-
gGlu-gGlu-gGlu-gGlu-Glu-gGlu-eLys(1,2);
(SEQ ID NO: 154)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-Glu-gGlu-gGlu-gGlu-gGlu-AHX(1,2);
(SEQ ID NO: 155)
Y-Aib-E-G-T-F-T-S-D-Y-S-I-Aib-L-D-E-Q-A-Q-Aib-A-F-V-R-H-L-I-N-K(1,3)-Aib-T-R-Q-R-Y-NH2.C20DA-
gGlu-gGlu-gGlu-gGlu-gGlu-eLys-eLys(1,2);
(SEQ ID NO: 156)
Y-Aib-E-G-T-F-T-S-D-Y-S-A-Aib-L-E-K-Q-A-Q-Aib-A-F-V-R-H-L-I-N-K(1,3)-Aib-T-R-Q-R-Y-
NH2.C18DA-gGlu-gGlu-gGlu-gGlu-gGlu-gGlu-eLys(1,2);
(SEQ ID NO: 157)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-Glu-gGlu-gGlu-gGlu-gGlu-AHX(1,2);
(SEQ ID NO: 158)
Y-Aib-E-G-T-F-T-S-D-Y-S-I-Aib-L-E-K-Q-A-Q-Aib-A-F-V-R-H-L-I-N-K(1,3)-L-T-R-Q-R-Y-NH2.C18DA-
gGlu-gGlu-gGlu-gGlu-gGlu(1,2);
(SEQ ID NO: 159)
Y-Aib-E-G-T-F-T-S-D-Y-S-I-Aib-L-E-K-Q-A-Q-Aib-A-F-V-R-H-L-I-N-K(1,3)-A-T-R-Q-R-Y-NH2.C18DA-
gGlu-gGlu-gGlu-Glu-gGlu-gGlu-eLys(1,2);
(SEQ ID NO: 160)
Y-Aib-E-G-T-F-T-S-D-Y-S-I-E-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 161)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C20DA-
gGlu-gGlu-gGlu-gGlu-gGlu-OEG-eLys(1,2);
(SEQ ID NO: 162)
Y-Aib-E-G-T-F-T-S-D-Y-S-I-Aib-L-E-K-Q-A-E-Aib-A-F-V-R-H-L-I-N-K(1,3)-Aib-T-R-Q-R-Y-NH2.C20DA-
gGlu-gGlu-gGlu-OEG-gGlu-gGlu-eLys(1,2);
(SEQ ID NO: 163)
Y-Aib-E-G-T-F-T-S-D-Y-S-I-Aib-L-E-K-Q-A-Q-Aib-E-F-V-R-H-L-I-N-K(1,3)-Aib-T-R-Q-R-Y-NH2.C18DA-
gGlu-gGlu-gGlu-gGlu-eLys(1,2);
(SEQ ID NO: 164)
Y-Aib-E-G-T-F-T-S-D-Y-S-I-Aib-L-E-K-E-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 165)
Y-Aib-E-G-T-F-T-S-D-Y-S-I-A-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-Glu-eLys(1,2);
(SEQ ID NO: 166)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C20DA-
gGlu-glu-gGlu-gGlu-eLys-gGlu-gGlu(1,2);
(SEQ ID NO: 167)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-Glu-gGlu-gGlu-AHX(1,2);
(SEQ ID NO: 168)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-eLys-gGlu-gGlu-gGlu(1,2);
(SEQ ID NO: 169)
Y-Aib-E-G-T-F-T-S-D-L-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 170)
Y-Aib-E-G-T-F-T-S-D-Y-S-I-Aib-L-E-K-Q-A-Q-E-A-F-V-R-H-L-I-N-K(1,3)-Aib-T-R-Q-R-Y-NH2.C18DA-
gGlu-gGlu-gGlu-gGlu-gGlu-gGlu-eLys(1,2);
(SEQ ID NO: 171)
Y-Aib-E-G-T-F-T-S-D-Y-S-I-Aib-L-E-R-Q-A-Q-Aib-A-F-V-R-H-L-I-N-K(1,3)-Aib-T-R-Q-R-Y-NH2.C20DA-
gGlu-gGlu-gGlu-gGlu-gGlu-gGlu-eLys(1,2);
(SEQ ID NO: 172)
Y-Aib-E-G-T-F-T-S-D-Y-S-I-Aib-L-E-K-Q-A-Q-Aib-A-F-V-R-H-L-I-N-K(1,3)-W-T-R-Q-R-Y-NH2.C18DA-
gGlu-gGlu-gGlu-gGlu-gGlu-gGlu-eLys(1,2);
(SEQ ID NO: 173)
Y-Aib-E-G-T-F-T-S-D-Y-S-I-Aib-L-D-K-Q-A-Q-Aib-A-F-V-R-H-L-L-N-K(1,3)-Aib-T-R-Q-R-Y-
NH2.C18DA-gGlu-gGlu-gGlu-gGlu-gGlu-gGlu-eLys(1,2);
(SEQ ID NO: 174)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C18DA-
gGlu-gGlu-gGlu-gGlu-gGlu-eLys-gGlu(1,2);
(SEQ ID NO: 175)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-gGlu-AHX-gGlu-AHX(1,2);
and
(SEQ ID NO: 176)
Y-Aib-E-G-T-F-T-S-D-Y-S-I-Aib-L-E-K-Q-A-Q-Aib-A-F-V-R-H-L-I-N-K(1,3)-Aib-T-R-Q-R-Y-NH2.C20DA-
gGlu-gGlu-gGlu-Glu-gGlu(1,2).

12. The compound according to claim 1, wherein the compound is in the form of a salt or in the form of a pharmaceutically acceptable salt.

13. A pharmaceutical composition comprising one or more pharmaceutically acceptable carriers and/or excipients and a therapeutically effective amount of a compound according to claim 1, or a pharmaceutically acceptable salt thereof.

14. A method for treating a disease or condition in a patient, wherein the method comprises administering to the patient in need thereof a therapeutically effective amount of a compound according to claim 1, or a pharmaceutically acceptable salt thereof, wherein the disease or condition is selected from the group consisting of:
overweight, chronic weight management, obesity, symptomatic obesity, eating disorders;
endocrine obesity, Cushing syndrome, hypothyroidism, insulinoma, obese type 2 diabetes, pseudohypoparathyroidism, hypogonadism;
obesity associated endocrine disorders, polycystic ovary syndrome (PCOS);
central obesity, hypothalamic obesity, frontal lobe syndrome, Kleine-Levin syndrome;
hereditary obesity, Prader-Willi syndrome, Laurence-Moon-Biedl syndrome;
drug-induced obesity, steroid-induced obesity, phenothiazine-induced obesity, insulin-induced obesity, sulfonylurea agent-induced obesity, β-blocker-induced obesity;
co-morbidities of obesity, co-morbidities of overweight, obesity associated type 2 diabetes, obesity associated high blood pressure, obesity associated metabolic dysfunction-associated fatty liver disease (MAFLD) or non-alcoholic fatty liver disease (NAFLD), obesity associated metabolic dysfunction-associated steatohepatitis (MASH) or non-alcoholic steato-hepatitis (NASH), obesity associated diabetic kidney disease (DKD), obesity associated chronic kidney disease (CKD), obesity associated osteoporosis, obesity associated sleep apnoe, obesity associated cancers, obesity associated asthma;
insulin resistance, diabetes, type 1 diabetes, type 2 diabetes, prediabetes;
hyperlipidemia, hypertriglyceridemia, hypercholesterolemia, high LDL-cholesterolemia, low HDL-cholesterolemia, postprandial hyperlipemia;
metabolic syndrome, abdominal obesity, high blood pressure, high blood sugar, high serum triglycerides, low serum high-density lipoprotein (HDL);
liver diseases, metabolic dysfunction-associated fatty liver disease (MAFLD), non-alcoholic fatty liver disease (NAFLD), metabolic dysfunction-associated steatohepatitis (MASH), non-alcoholic steato-hepatitis (NASH), portal hypertension;
cardiovascular diseases, hypertension, atherosclerosis, stroke, cardiac failure;
kidney diseases, diabetic kidney disease (DKD), chronic kidney disease (CKD);
neurodegenerative diseases, Alzheimer's disease, and Parkinson's disease.
